SEQUENCE LISTING (148 sequences)

SEQ ID NO: 1 - protein, 129 aa, Homo sapiens [sequence omitted]

SEQ ID NO: 2 - RNA, 387 nt, Homo sapiens
  misc_feature 1..387: note = Human IL-4 mRNA
  source 1..387: mol_type = mRNA
cacaagtgcgacatcaccctgcaggagatcatcaagaccctgaacagcctgaccgagcagaagaccctgtgcaccgagctgaccgtgaccgacatcttcgccgccagcaagaacaccaccgagaaggagaccttctgccgcgccgccaccgtgctgcgccagttctacagccaccacgagaaggacacccgctgcctgggcgccaccgcccagcagttccaccgccacaagcagctgatccgcttcctgaagcgcctggaccgcaacctgtggggcctggccggcctgaacagctgccccgtgaaggaggccaaccagagcaccctggagaacttcctggagcgcctgaagaccatcatgcgcgagaagtacagcaagtgcagcagc

SEQ ID NO: 3 - protein, 22 aa, synthetic construct
  REGION 1..22: note = Human IL-4 epitope
LQEIIKTLNSLTEQKTLCTELT

SEQ ID NO: 4 - protein, 15 aa, synthetic construct
  REGION 1..15: note = Human IL-4 epitope
KETFCRAATVLRQFY

SEQ ID NO: 5 - protein, 26 aa, synthetic construct
  REGION 1..26: note = Human IL-4 epitope
AQQFHRHKQLIRFLKRLDRNLWGLAG

SEQ ID NO: 6 - protein, 19 aa, synthetic construct
  REGION 1..19: note = Human IL-4 epitope
LENFLERLKTIMREKYSKC

SEQ ID NO: 7 - protein, 24 aa, synthetic construct
  REGION 1..24: note = Human IL-4 cyclic peptide
CLQEIIKTLNSLTEQKTLCTELTC

SEQ ID NO: 8 - protein, 17 aa, synthetic construct
  REGION 1..17: note = Human IL-4 cyclic peptide
CKETFCRAATVLRQFYC

SEQ ID NO: 9 - protein, 28 aa, synthetic construct
  REGION 1..28: note = Human IL-4 cyclic peptide
CAQQFHRHKQLIRFLKRLDRNLWGLAGC

SEQ ID NO: 10 - protein, 20 aa, synthetic construct
  REGION 1..20: note = Human IL-4 cyclic peptide
CLENFLERLKTIMREKYSKC

SEQ ID NO: 11 - protein, 120 aa, Mus musculus
  REGION 1..120: note = Murine IL-4 protein
HIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 12 - RNA, 360 nt, Mus musculus
  misc_feature 1..360: note = Murine IL-4 mRNA
  source 1..360: mol_type = mRNA
cacatccacggctgcgacaagaaccacctgagggagatcatcggcatcctgaacgaggtgaccggcgagggcaccccctgcaccgagatggacgtgcccaacgtgctgaccgccaccaagaacaccaccgagagcgagctggtgtgcagggccagcaaggtgctgaggatcttctacctgaagcacggcaagaccccctgcctgaagaagaacagcagcgtgctgatggagctgcagaggctgttcagggccttcaggtgcctggacagcagcatcagctgcaccatgaacgagagcaagagcaccagcctgaaggacttcctggagagcctgaagagcatcatgcagatggactacagc

SEQ ID NO: 13 - protein, 22 aa, synthetic construct
  REGION 1..22: note = Murine IL-4 epitope
LREIIGILNEVTGEGTPCTEMD

SEQ ID NO: 14 - protein, 15 aa, synthetic construct
  REGION 1..15: note = Murine IL-4 epitope
SELVCRASKVLRIFY

SEQ ID NO: 15 - protein, 19 aa, synthetic construct
  REGION 1..19: note = Murine IL-4 epitope
SSVLMELQRLFRAFRCLDS

SEQ ID NO: 16 - protein, 17 aa, synthetic construct
  REGION 1..17: note = Murine IL-4 epitope
LKDFLESLKSIMQMDYS

SEQ ID NO: 17 - protein, 24 aa, synthetic construct
  REGION 1..24: note = Murine IL-4 cyclic peptide
CLREIIGILNEVTGEGTPCTEMDC

SEQ ID NO: 18 - protein, 17 aa, synthetic construct
  REGION 1..17: note = Murine IL-4 cyclic peptide
CSELVCRASKVLRIFYC

SEQ ID NO: 19 - protein, 21 aa, synthetic construct
  REGION 1..21: note = Murine IL-4 cyclic peptide
CSSVLMELQRLFRAFRCLDSC

SEQ ID NO: 20 - protein, 19 aa, synthetic construct
  REGION 1..19: note = Murine IL-4 cyclic peptide
CLKDFLESLKSIMQMDYSC

SEQ ID NO: 21 - protein, 108 aa, Canis lupus
  REGION 1..108: note = Canine IL-4 protein
  source 1..108: note = sub_species: Canis lupus familiaris
HNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRH

SEQ ID NO: 22 - RNA, 324 nt, Canis lupus
  misc_feature 1..324: note = Canine IL-4 mRNA
  source 1..324: mol_type = mRNA; sub_species = Canis lupus familiaris
cacaacttcaacatcaccatcaaggagatcatcaagatgctgaacatcctgaccgcccgcaacgacagctgcatggagctgaccgtgaaggacgtgttcaccgcccccaagaacaccagcgacaaggagatcttctgccgcgccgccaccgtgctgcgccagatctacacccacaactgcagcaaccgctacctgcgcggcctgtaccgcaacctgagcagcatggccaacaagacctgcagcatgaacgagatcaagaagagcaccctgaaggacttcctggagcgcctgaaggtgatcatgcagaagaagtactaccgccac

SEQ ID NO: 23 - protein, 122 aa, Homo sapiens
  REGION 1..122: note = Human IL-13 protein
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 24 - RNA, 366 nt, Homo sapiens
  misc_feature 1..366: note = Human IL-13 mRNA
  source 1..366: mol_type = mRNA
ctgacctgcctgggcggcttcgccagccccggccccgtgccccccagcaccgccctgcgcgagctgatcgaggagctggtgaacatcacccagaaccagaaggcccccctgtgcaacggcagcatggtgtggagcatcaacctgaccgccggcatgtactgcgccgccctggagagcctgatcaacgtgagcggctgcagcgccatcgagaagacccagcgcatgctgagcggcttctgcccccacaaggtgagcgccggccagttcagcagcctgcacgtgcgcgacaccaagatcgaggtggcccagttcgtgaaggacctgctgctgcacctgaagaagctgttccgcgagggccgcttcaac

SEQ ID NO: 25 - protein, 22 aa, synthetic construct
  REGION 1..22: note = Human IL-13 epitope
LRELIEELVNITQNQKAPLCNG

SEQ ID NO: 26 - protein, 17 aa, synthetic construct
  REGION 1..17: note = Human IL-13 epitope
NGSMVWSINLTAGMYCA

SEQ ID NO: 27 - protein, 22 aa, synthetic construct
  REGION 1..22: note = Human IL-13 epitope
AGMYCAALESLINVSGCSAIEK

SEQ ID NO: 28 - protein, 22 aa, synthetic construct
  REGION 1..22: note = Human IL-13 epitope
VAQFVKDLLLHLKKLFREGRFN

SEQ ID NO: 29 - protein, 24 aa, synthetic construct
  REGION 1..24: note = Human IL-13 cyclic peptide
CLRELIEELVNITQNQKAPLCNGC

SEQ ID NO: 30 - protein, 19 aa, synthetic construct
  REGION 1..19: note = Human IL-13 cyclic peptide
CNGSMVWSINLTAGMYCAC

SEQ ID NO: 31 - protein, 24 aa, synthetic construct
  REGION 1..24: note = Human IL-13 cyclic peptide
CAGMYCAALESLINVSGCSAIEKC

SEQ ID NO: 32 - protein, 24 aa, synthetic construct
  REGION 1..24: note = Human IL-13 cyclic peptide
CVAQFVKDLLLHLKKLFREGRFNC

SEQ ID NO: 33 - protein, 113 aa, Mus musculus
  REGION 1..113: note = Murine IL-13 protein
APGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPF

SEQ ID NO: 34 - RNA, 339 nt, Mus musculus
  misc_feature 1..339: note = Murine IL-13 mRNA
  source 1..339: mol_type = mRNA
gcccccggccccgtgcccaggagcgtgagcctgcccctgaccctgaaggagctgatcgaggagctgagcaacatcacccaggaccagacccccctgtgcaacggcagcatggtgtggagcgtggacctggccgccggcggcttctgcgtggccctggacagcctgaccaacatcagcaactgcaacgccatctacaggacccagaggatcctgcacggcctgtgcaacaggaaggcccccaccaccgtgagcagcctgcccgacaccaagatcgaggtggcccacttcatcaccaagctgctgagctacaccaagcagctgttcaggcacggccccttc

SEQ ID NO: 35 - protein, 21 aa, synthetic construct
  REGION 1..21: note = Murine IL-13 epitope
LKELIEELSNITQDQTPLCNG

SEQ ID NO: 36 - protein, 17 aa, synthetic construct
  REGION 1..17: note = Murine IL-13 epitope
NGSMVWSVDLAAGGFCV

SEQ ID NO: 37 - protein, 22 aa, synthetic construct
  REGION 1..22: note = Murine IL-13 epitope
AGGFCVALDSLTNISNCNAIYR

SEQ ID NO: 38 - protein, 21 aa, synthetic construct
  REGION 1..21: note = Murine IL-13 epitope
VAHFITKLLSYTKQLFRHGPF

SEQ ID NO: 39 - protein, 23 aa, synthetic construct
  REGION 1..23: note = Murine IL-13 cyclic peptide
CLKELIEELSNITQDQTPLCNGC

SEQ ID NO: 40 - protein, 19 aa, synthetic construct
  REGION 1..19: note = Murine IL-13 cyclic peptide
CNGSMVWSVDLAAGGFCVC

SEQ ID NO: 41 - protein, 24 aa, synthetic construct
  REGION 1..24: note = Murine IL-13 cyclic peptide
CAGGFCVALDSLTNISNCNAIYRC

SEQ ID NO: 42 - protein, 23 aa, synthetic construct
  REGION 1..23: note = Murine IL-13 cyclic peptide
CVAHFITKLLSYTKQLFRHGPFC

SEQ ID NO: 43 - protein, 113 aa, Canis lupus
  REGION 1..113: note = Canine IL-13 protein
  source 1..113: note = sub_species: Canis lupus familiaris
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 44 - RNA, 339 nt, Canis lupus
  misc_feature 1..339: note = Canine IL-13 mRNA
  source 1..339: mol_type = mRNA; sub_species = Canis lupus familiaris
agccccagccccgtgacccccagccccaccctgaaggagctgatcgaggagctggtgaacatcacccagaaccaggccagcctgtgcaacggcagcatggtgtggagcgtgaacctgaccgccggcatgtactgcgccgccctggagagcctgatcaacgtgagcgactgcagcgccatccagcgcacccagcgcatgctgaaggccctgtgcagccagaagcccgccgccggccagatcagcagcgagcgcagccgcgacaccaagatcgaggtgatccagctggtgaagaacctgctgacctacgtgcgcggcgtgtaccgccacggcaacttccgc

SEQ ID NO: 45 - protein, 5 aa, synthetic construct
  REGION 1..5: note = Spacer
GPGPG

SEQ ID NO: 46 - protein, 150 aa, synthetic construct
  REGION 1..150: note = CRM197 protein from amino acid 300 to 450
TAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHI

SEQ ID NO: 47 - RNA, 450 nt, synthetic construct
  misc_feature 1..450: note = mRNA encoding CRM197 protein from amino acid 300 to 450
  source 1..450: mol_type = other RNA
accgccgccctgagcatcctgcccggcatcggcagcgtgatgggcatcgccgacggcgccgtgcaccacaacaccgaggagatcgtggcccagagcatcgccctgagcagcctgatggtggcccaggccatccccctggtgggcgagctggtggacatcggcttcgccgcctacaacttcgtggagagcatcatcaacctgttccaggtggtgcacaacagctacaacaggcccgcctacagccccggccacaagacccagcccttcctgcacgacggctacgccgtgagctggaacaccgtggaggacagcatcatcaggaccggcttccagggcgagagcggccacgacatcaagatcaccgccgagaacacccccctgcccatcgccggcgtgctgctgcccaccatccccggcaagctggacgtgaacaagagcaagacccacatc

SEQ ID NO: 48 - protein, 535 aa, synthetic construct
  REGION 1..535: note = CRM197 protein
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 49 - RNA, 1605 nt, synthetic construct
  misc_feature 1..1605: note = CRM197 mRNA
  source 1..1605: mol_type = other RNA
ggcgccgacgacgtggtggacagcagcaagagcttcgtgatggagaacttcagcagctaccacggcaccaagcccggctacgtggacagcatccagaagggcatccagaagcccaagagcggcacccagggcaactacgacgacgactggaaggagttctacagcaccgacaacaagtacgacgccgccggctacagcgtggacaacgagaaccccctgagcggcaaggccggcggcgtggtgaaggtgacctaccccggcctgaccaaggtgctggccctgaaggtggacaacgccgagaccatcaagaaggagctgggcctgagcctgaccgagcccctgatggagcaggtgggcaccgaggagttcatcaagaggttcggcgacggcgccagcagggtggtgctgagcctgcccttcgccgagggcagcagcagcgtggagtacatcaacaactgggagcaggccaaggccctgagcgtggagctggagatcaacttcgagaccaggggcaagaggggccaggacgccatgtacgagtacatggcccaggcctgcgccggcaacagggtgaggaggagcgtgggcagcagcctgagctgcatcaacctggactgggacgtgatcagggacaagaccaagaccaagatcgagagcctgaaggagcacggccccatcaagaacaagatgagcgagagccccaacaagaccgtgagcgaggagaaggccaagcagtacctggaggagttccaccagaccgccctggagcaccccgagctgagcgagctgaagaccgtgaccggcaccaaccccgtgttcgccggcgccaactacgccgcctgggccgtgaacgtggcccaggtgatcgacagcgagaccgccgacaacctggagaagaccaccgccgccctgagcatcctgcccggcatcggcagcgtgatgggcatcgccgacggcgccgtgcaccacaacaccgaggagatcgtggcccagagcatcgccctgagcagcctgatggtggcccaggccatccccctggtgggcgagctggtggacatcggcttcgccgcctacaacttcgtggagagcatcatcaacctgttccaggtggtgcacaacagctacaacaggcccgcctacagccccggccacaagacccagcccttcctgcacgacggctacgccgtgagctggaacaccgtggaggacagcatcatcaggaccggcttccagggcgagagcggccacgacatcaagatcaccgccgagaacacccccctgcccatcgccggcgtgctgctgcccaccatccccggcaagctggacgtgaacaagagcaagacccacatcagcgtgaacggcaggaagatcaggatgaggtgcagggccatcgacggcgacgtgaccttctgcaggcccaagagccccgtgtacgtgggcaacggcgtgcacgccaacctgcacgtggccttccacaggagcagcagcgaaaagatccacagcaacgagatcagcagcgacagcatcggcgtgctgggctaccagaagaccgtggaccacaccaaggtgaacagcaagctgagcctgttcttcgagatcaagagc

SEQ ID NO: 50 - protein, 14 aa, synthetic construct
  REGION 1..14: note = CRM197 protein from amino acid 299 to 312
KTTAALSILPGIGS

SEQ ID NO: 51 - RNA, 42 nt, synthetic construct
  misc_feature 1..42: note = mRNA encoding CRM197 protein from amino acid 299 to 312
  source 1..42: mol_type = other RNA
aagaccaccgccgccctgagcatcctgcccggcatcggcagc

SEQ ID NO: 52 - protein, 16 aa, synthetic construct
  REGION 1..16: note = CRM197 protein from amino acid 425 to 440
TPLPIAGVLLPTIPGK

SEQ ID NO: 53 - RNA, 48 nt, synthetic construct
  misc_feature 1..48: note = mRNA encoding CRM197 protein from amino acid 425 to 440
  source 1..48: mol_type = other RNA
acccccctgcccatcgccggcgtgctgctgcccaccatccccggcaag

SEQ ID NO: 54 - protein, 32 aa, synthetic construct
  REGION 1..32: note = TpD protein
ILMQYIKANSKFIGIPMGLPQSIALSSLMVAQ

SEQ ID NO: 55 - RNA, 96 nt, synthetic construct
  misc_feature 1..96: note = TpD mRNA
  source 1..96: mol_type = other RNA
atcctgatgcagtacatcaaggccaacagcaagttcatcggcatccccatgggcctgccccagagcatcgccctgagcagcctgatggtggcccag

SEQ ID NO: 56 - protein, 39 aa, synthetic construct
  REGION 1..39: note = P2-RR-p30 Tetanus toxin protein
QYIKANSKFIGITERRFNNFTVSFWLRVPKVSASHLEQY

SEQ ID NO: 57 - RNA, 117 nt, synthetic construct
  misc_feature 1..117: note = P2-RR-p30 Tetanus toxin mRNA
  source 1..117: mol_type = other RNA
cagtacatcaaggccaacagcaagttcatcggcatcaccgagaggaggttcaacaacttcaccgtgagcttctggctgagggtgcccaaggtgagcgccagccacctggagcagtac

SEQ ID NO: 58 - protein, 15 aa, synthetic construct
  REGION 1..15: note = p23 Tetanus toxin protein
VSIDKFRIFCKANPK

SEQ ID NO: 59 - protein, 16 aa, synthetic construct
  REGION 1..16: note = p32 Tetanus toxin protein
LKFIIKRYTPNNEIDS

SEQ ID NO: 60 - protein, 16 aa, synthetic construct
  REGION 1..16: note = p21 Tetanus toxin protein
IREDNNITLKLDRCNN

SEQ ID NO: 61 - protein, 18 aa, synthetic construct
  REGION 1..18: note = P. falciparum circumsporozoite protein
EKKIAKMEKASSVFNVVN

SEQ ID NO: 62 - protein, 21 aa, synthetic construct
  REGION 1..21: note = p30 Tetanus toxin protein
FNNFTVSFWLRVPKVSASHLE

SEQ ID NO: 63 - protein, 14 aa, synthetic construct
  REGION 1..14: note = p2 Tetanus toxin protein
QYIKANSKFIGITE

SEQ ID NO: 64 - protein, 20 aa, synthetic construct
  REGION 1..20: note = HBV nucleocapsid protein
PHHTALRQAILCWGELMTLA

SEQ ID NO: 65 - protein, 13 aa, synthetic construct
  REGION 1..13: note = Influenza virus hemagglutinin protein
PKYVKQNTLKLAT

SEQ ID NO: 66 - protein, 15 aa, synthetic construct
  REGION 1..15: note = HBV Surface antigen protein
FFLLTRILTIPQSLD

SEQ ID NO: 67 - protein, 16 aa, synthetic construct
  REGION 1..16: note = Influenza virus Matrix protein
YSGPLKAEIAQRLEDV

SEQ ID NO: 68 - RNA, 45 nt, synthetic construct
  misc_feature 1..45: note = p23 Tetanus toxin mRNA
  source 1..45: mol_type = other RNA
gtgagcatcgacaagttcaggatcttctgcaaggccaaccccaag

SEQ ID NO: 69 - RNA, 48 nt, synthetic construct
  misc_feature 1..48: note = p32 Tetanus toxin mRNA
  source 1..48: mol_type = other RNA
ctgaagttcatcatcaagaggtacacccccaacaacgagatcgacagc

SEQ ID NO: 70 - RNA, 48 nt, synthetic construct
  misc_feature 1..48: note = p21 Tetanus toxin mRNA
  source 1..48: mol_type = other RNA
atcagggaggacaacaacatcaccctgaagctggacaggtgcaacaac

SEQ ID NO: 71 - RNA, 54 nt, synthetic construct
  misc_feature 1..54: note = P. falciparum circumsporozoite mRNA
  source 1..54: mol_type = other RNA
gaaaagaagatcgccaagatggagaaggccagcagcgtgttcaacgtggtgaac

SEQ ID NO: 72 - RNA, 63 nt, synthetic construct
  misc_feature 1..63: note = p30 Tetanus toxin mRNA
  source 1..63: mol_type = other RNA
ttcaacaacttcaccgtgagcttctggctgagggtgcccaaggtgagcgccagccacctggag

SEQ ID NO: 73 - RNA, 42 nt, synthetic construct
  misc_feature 1..42: note = p2 Tetanus toxin mRNA
  source 1..42: mol_type = other RNA
cagtacatcaaggccaacagcaagttcatcggcatcaccgaa

SEQ ID NO: 74 - RNA, 60 nt, synthetic construct
  misc_feature 1..60: note = HBV nucleocapsid mRNA
  source 1..60: mol_type = other RNA
ccccaccacaccgccctgaggcaggccatcctgtgctggggcgagctgatgaccctggcc

SEQ ID NO: 75 - RNA, 39 nt, synthetic construct
  misc_feature 1..39: note = Influenza virus hemagglutinin mRNA
  source 1..39: mol_type = other RNA
cccaagtacgtgaagcagaacaccctgaagctggccacc

SEQ ID NO: 76 - RNA, 45 nt, synthetic construct
  misc_feature 1..45: note = HBV Surface antigen mRNA
  source 1..45: mol_type = other RNA
ttcttcctgctgaccaggatcctgaccatcccccagagcctggac

SEQ ID NO: 77 - RNA, 48 nt, synthetic construct
  misc_feature 1..48: note = Influenza virus Matrix mRNA
  source 1..48: mol_type = other RNA
tacagcggccccctgaaggccgagatcgcccagaggctggaggacgtg

SEQ ID NO: 78 - protein, 24 aa, synthetic construct
  REGION 1..24: note = Canine IL-4 signal peptide
MGLTSQLIPTLVCLLALTSTFVHG

SEQ ID NO: 79 - RNA, 72 nt, synthetic construct
  misc_feature 1..72: note = Canine IL-4 signal peptide mRNA
  source 1..72: mol_type = other RNA
atgggcctgaccagccagctgatccccaccctggtgtgcctgctggccctgaccagcaccttcgtgcacggc

SEQ ID NO: 80 - protein, 182 aa, synthetic construct
  REGION 1..182: note = N10 protein
VSIDKFRIFCKANPKKGLKFIIKRYTPNNEIDSKGIREDNNITLKLDRCNNKGEKKIAKMEKASSVFNVVNKGFNNFTVSFWLRVPKVSASHLEKGQYIKANSKFIGITEKGPHHTALRQAILCWGELMTLAKGPKYVKQNTLKLATKGFFLLTRILTIPQSLDKGYSGPLKAEIAQRLEDV

SEQ ID NO: 81 - RNA, 546 nt, synthetic construct
  misc_feature 1..546: note = N10 mRNA
  source 1..546: mol_type = other RNA
gtgagcatcgacaagttcaggatcttctgcaaggccaaccccaagaagggcctgaagttcatcatcaagaggtacacccccaacaacgagatcgacagcaagggcatcagggaggacaacaacatcaccctgaagctggacaggtgcaacaacaagggcgaaaagaagatcgccaagatggagaaggccagcagcgtgttcaacgtggtgaacaagggcttcaacaacttcaccgtgagcttctggctgagggtgcccaaggtgagcgccagccacctggagaagggccagtacatcaaggccaacagcaagttcatcggcatcaccgaaaagggcccccaccacaccgccctgaggcaggccatcctgtgctggggcgagctgatgaccctggccaagggccccaagtacgtgaagcagaacaccctgaagctggccaccaagggcttcttcctgctgaccaggatcctgaccatcccccagagcctggacaagggctacagcggccccctgaaggccgagatcgcccagaggctggaggacgtg

SEQ ID NO: 82 - protein, 350 aa, synthetic construct
  REGION 1..350: note = N19 protein
VSIDKFRIFCKANPKKGLKFIIKRYTPNNEIDSKGIREDNNITLKLDRCNNKGEKKIAKMEKASSVFNVVNKGFNNFTVSFWLRVPKVSASHLEKGQYIKANSKFIGITEKGPHHTALRQAILCWGELMTLAKGPKYVKQNTLKLATKGFFLLTRILTIPQSLDKGYSGPLKAEIAQRLEDVKGVSIDKFRIFCKANPKKGLKFIIKRYTPNNEIDSKGIREDNNITLKLDRCNNKGEKKIAKMEKASSVFNVVNKGFNNFTVSFWLRVPKVSASHLEKGQYIKANSKFIGITEKGPHHTALRQAILCWGELMTLAKGPKYVKQNTLKLATKGFFLLTRILTIPQSLDKG

SEQ ID NO: 83 - RNA, 1050 nt, synthetic construct
  source 1..1050: mol_type = other RNA
  misc_feature 1..1050: note = N19 mRNA
gtgagcatcgacaagttcaggatcttctgcaaggccaaccccaagaagggcctgaagttcatcatcaagaggtacacccccaacaacgagatcgacagcaagggcatcagggaggacaacaacatcaccctgaagctggacaggtgcaacaacaagggcgaaaagaagatcgccaagatggagaaggccagcagcgtgttcaacgtggtgaacaagggcttcaacaacttcaccgtgagcttctggctgagggtgcccaaggtgagcgccagccacctggagaagggccagtacatcaaggccaacagcaagttcatcggcatcaccgaaaagggcccccaccacaccgccctgaggcaggccatcctgtgctggggcgagctgatgaccctggccaagggccccaagtacgtgaagcagaacaccctgaagctggccaccaagggcttcttcctgctgaccaggatcctgaccatcccccagagcctggacaagggctacagcggccccctgaaggccgagatcgcccagaggctggaggacgtgaagggcgtgagcatcgacaagttcaggatcttctgcaaggccaaccccaagaagggcctgaagttcatcatcaagaggtacacccccaacaacgagatcgacagcaagggcatcagggaggacaacaacatcaccctgaagctggacaggtgcaacaacaagggcgaaaagaagatcgccaagatggagaaggccagcagcgtgttcaacgtggtgaacaagggcttcaacaacttcaccgtgagcttctggctgagggtgcccaaggtgagcgccagccacctggagaagggccagtacatcaaggccaacagcaagttcatcggcatcaccgaaaagggcccccaccacaccgccctgaggcaggccatcctgtgctggggcgagctgatgaccctggccaagggccccaagtacgtgaagcagaacaccctgaagctggccaccaagggcttcttcctgctgaccaggatcctgaccatcccccagagcctggacaagggc

SEQ ID NO: 84 - protein, 23 aa, synthetic construct
  REGION 1..23: note = Equine IL-4 signal peptide
MGLTYQLLPALVCLLACTSFIQG

SEQ ID NO: 85 - RNA, 69 nt, synthetic construct
  misc_feature 1..69: note = Equine IL-4 signal peptide mRNA
  source 1..69: mol_type = other RNA
atgggcctgacctaccagctgctgcccgccctggtgtgcctgctggcctgcaccagcttcatccagggc

SEQ ID NO: 86 - protein, 5 aa, synthetic construct
  REGION 1..5: note = PMGLP spacer protein
PMGLP

SEQ ID NO: 87 - RNA, 15 nt, synthetic construct
  misc_feature 1..15: note = PMGLP spacer mRNA
  source 1..15: mol_type = other RNA
cccatgggcctgccc

SEQ ID NO: 88 - protein, 18 aa, synthetic construct
  REGION 1..18: note = Signal peptide
MKWVTFISLLFLFSSAYS

SEQ ID NO: 89 - protein, 14 aa, synthetic construct
  REGION 1..14: note = Signal peptide
MQLLSCIALILALV

SEQ ID NO: 90 - protein, 17 aa, synthetic construct
  REGION 1..17: note = Signal peptide
MGVKVLFALICIAVAEA

SEQ ID NO: 91 - protein, 18 aa, synthetic construct
  REGION 1..18: note = Signal peptide
MAFLWLLSCWALLGTTFG

SEQ ID NO: 92 - protein, 21 aa, synthetic construct
  REGION 1..21: note = Signal peptide
MWWRLWWLLLLLLLLWPMVWA

SEQ ID NO: 93 - protein, 15 aa, synthetic construct
  REGION 1..15: note = Signal peptide
MNLLLILTFVAAAVA

SEQ ID NO: 94 - protein, 22 aa, synthetic construct
  REGION 1..22: note = Canine IL-4 epitope
IKEIIKMLNILTARNDSCMELT

SEQ ID NO: 95 - protein, 15 aa, synthetic construct
  REGION 1..15: note = Canine IL-4 epitope
KEIFCRAATVLRQIY

SEQ ID NO: 96 - protein, 15 aa, synthetic construct
  REGION 1..15: note = Canine IL-4 epitope
RYLRGLYRNLSSMAN

SEQ ID NO: 97 - protein, 16 aa, synthetic construct
  REGION 1..16: note = Canine IL-4 epitope
LKDFLERLKVIMQKKY

SEQ ID NO: 98 - protein, 10 aa, synthetic construct
  REGION 1..10: note = Spacer
GGSGGGGSGG

SEQ ID NO: 99 - protein, 111 aa, Equus caballus
  REGION 1..111: note = Equine IL-4 protein
CKYDITLQEIIKTLNLTDGKGKNSCMELTVADAFGPKNTDGKEICRAAKVLQQYKRHDRSLIKECLSGLDRNLKGMANGTCCTVNEAKKSTLKDFLERLKTIMKEKYSKCS

SEQ ID NO: 100 - RNA, 333 nt, Equus caballus
  misc_feature 1..333: note = Equine IL-4 mRNA
  source 1..333: mol_type = mRNA
tgcaagtacgacatcaccctgcaggagatcatcaagaccctgaacctgaccgacggcaagggcaagaacagctgcatggagctgaccgtggccgacgccttcggccccaagaacaccgacggcaaggagatctgccgcgccgccaaggtgctgcagcagtacaagcgccacgaccgcagcctgatcaaggagtgcctgagcggcctggaccgcaacctgaagggcatggccaacggcacctgctgcaccgtgaacgaggccaagaagagcaccctgaaggacttcctggagcgcctgaagaccatcatgaaggagaagtacagcaagtgcagc

SEQ ID NO: 101 - protein, 113 aa, Equus caballus
  REGION 1..113: note = Equine IL-13 protein
APLPSSMALKELIKELVNITQNQAPLCNGSMVWSVNLTADTYCRALESLSNVSTCSAIQNTRKMLTKLCPHQLSAGQVSSERARDTKIEVIVLVKDLLKNLRKIFHGGKHVDA

SEQ ID NO: 102 - RNA, 339 nt, Equus caballus
  misc_feature 1..339: note = Equine IL-13 mRNA
  source 1..339: mol_type = mRNA
gcccccctgcccagcagcatggccctgaaggagctgatcaaggagctggtgaacatcacccagaaccaggcccccctgtgcaacggcagcatggtgtggagcgtgaacctgaccgccgacacctactgccgcgccctggagagcctgagcaacgtgagcacctgcagcgccatccagaacacccgcaagatgctgaccaagctgtgcccccaccagctgagcgccggccaggtgagcagcgagcgcgcccgcgacaccaagatcgaggtgatcgtgctggtgaaggacctgctgaagaacctgcgcaagatcttccacggcggcaagcacgtggacgcc

SEQ ID NO: 103 - protein, 24 aa, synthetic construct
  REGION 1..24: note = Human IL-4 signal peptide
MGLTSQLLPPLFFLLACAGNFVHG

SEQ ID NO: 104 - RNA, 72 nt, synthetic construct
  misc_feature 1..72: note = Human IL-4 signal peptide mRNA
  source 1..72: mol_type = other RNA
atgggcctgaccagccagctgctgccccccctgttcttcctgctggcctgcgccggcaacttcgtgcacggc

SEQ ID NO: 105 - protein, 24 aa, synthetic construct
  REGION 1..24: note = Canine IL-4 cyclic epitope
CIKEIIKMLNILTARNDSCMELTC

SEQ ID NO: 106 - protein, 17 aa, synthetic construct
  REGION 1..17: note = Canine IL-4 cyclic epitope
CKEIFCRAATVLRQIYC

SEQ ID NO: 107 - protein, 17 aa, synthetic construct
  REGION 1..17: note = Canine IL-4 cyclic epitope
CRYLRGLYRNLSSMANC

SEQ ID NO: 108 - protein, 18 aa, synthetic construct
  REGION 1..18: note = Canine IL-4 cyclic epitope
CLKDFLERLKVIMQKKYC

SEQ ID NO: 109 - protein, 23 aa, synthetic construct
  REGION 1..23: note = Equine IL-4 epitope
LQEIIKTLNLTDGKGKNSCMELT

SEQ ID NO: 110 - protein, 14 aa, synthetic construct
  REGION 1..14: note = Equine IL-4 epitope
KEICRAAKVLQQYK

SEQ ID NO: 111 - protein, 20 aa, synthetic construct
  REGION 1..20: note = Equine IL-4 epitope
RSLIKECLSGLDRNLKGMAN

SEQ ID NO: 112 - protein, 19 aa, synthetic construct
  REGION 1..19: note = Equine IL-4 epitope
LKDFLERLKTIMKEKYSKC

SEQ ID NO: 113 - protein, 25 aa, synthetic construct
  REGION 1..25: note = Equine IL-4 cyclic epitope
CLQEIIKTLNLTDGKGKNSCMELTC

SEQ ID NO: 114 - protein, 16 aa, synthetic construct
  REGION 1..16: note = Equine IL-4 cyclic epitope
CKEICRAAKVLQQYKC

SEQ ID NO: 115 - protein, 22 aa, synthetic construct
  REGION 1..22: note = Equine IL-4 cyclic epitope
CRSLIKECLSGLDRNLKGMANC

SEQ ID NO: 116 - protein, 20 aa, synthetic construct
  REGION 1..20: note = Equine IL-4 cyclic epitope
CLKDFLERLKTIMKEKYSKC

SEQ ID NO: 117 - protein, 24 aa, synthetic construct
  REGION 1..24: note = Human IL-13 signal peptide
MHPLLNPLLLALGLMALLLTTVIA

SEQ ID NO: 118 - RNA, 72 nt, synthetic construct
  misc_feature 1..72: note = Human IL-13 signal peptide mRNA
  source 1..72: mol_type = other RNA
atgcaccccctgctgaaccccctgctgctggccctgggcctgatggccctgctgctgaccaccgtgatcgcc

SEQ ID NO: 119 - protein, 21 aa, synthetic construct
  REGION 1..21: note = Canine IL-13 epitope
LKELIEELVNITQNQASLCNG

SEQ ID NO: 120 - protein, 17 aa, synthetic construct
  REGION 1..17: note = Canine IL-13 epitope
NGSMVWSVNLTAGMYCA

SEQ ID NO: 121 - protein, 22 aa, synthetic construct
  REGION 1..22: note = Canine IL-13 epitope
AGMYCAALESLINVSDCSAIQR

SEQ ID NO: 122 - protein, 21 aa, synthetic construct
  REGION 1..21: note = Canine IL-13 epitope
VIQLVKNLLTYVRGVYRHGNF

SEQ ID NO: 123 - protein, 23 aa, synthetic construct
  REGION 1..23: note = Canine IL-13 cyclic epitope
CLKELIEELVNITQNQASLCNGC

SEQ ID NO: 124 - protein, 19 aa, synthetic construct
  REGION 1..19: note = Canine IL-13 cyclic epitope
CNGSMVWSVNLTAGMYCAC

SEQ ID NO: 125 - protein, 24 aa, synthetic construct
  REGION 1..24: note = Canine IL-13 cyclic epitope
CAGMYCAALESLINVSDCSAIQRC

SEQ ID NO: 126 - protein, 23 aa, synthetic construct
  REGION 1..23: note = Canine IL-13 cyclic epitope
CVIQLVKNLLTYVRGVYRHGNFC

SEQ ID NO: 127 - protein, 18 aa, synthetic construct
  REGION 1..18: note = Canine IL-13 signal peptide
MALWLTVVIALTCLGGLA

SEQ ID NO: 128 - RNA, 54 nt, synthetic construct
  misc_feature 1..54: note = Canine IL-13 signal peptide mRNA
  source 1..54: mol_type = other RNA
atggccctgtggctgaccgtggtgatcgccctgacctgcctgggcggcctggcc

SEQ ID NO: 129 - protein, 21 aa, synthetic construct
  REGION 1..21: note = Equine IL-13 epitope
LKELIKELVNITQNQAPLCNG

SEQ ID NO: 130 - protein, 18 aa, synthetic construct
  REGION 1..18: note = Equine IL-13 epitope
NGSMVWSVNLTADTYCRA

SEQ ID NO: 131 - protein, 22 aa, synthetic construct
  REGION 1..22: note = Equine IL-13 epitope
ADTYCRALESLSNVSTCSAIQN

SEQ ID NO: 132 - protein, 20 aa, synthetic construct
  REGION 1..20: note = Equine IL-13 epitope
VIVLVKDLLKNLRKIFHGGK

SEQ ID NO: 133 - protein, 23 aa, synthetic construct
  REGION 1..23: note = Equine IL-13 cyclic epitope
CLKELIKELVNITQNQAPLCNGC

SEQ ID NO: 134 - protein, 20 aa, synthetic construct
  REGION 1..20: note = Equine IL-13 cyclic epitope
CNGSMVWSVNLTADTYCRAC

SEQ ID NO: 135 - protein, 24 aa, synthetic construct
  REGION 1..24: note = Equine IL-13 cyclic epitope
CADTYCRALESLSNVSTCSAIQNC

SEQ ID NO: 136 - protein, 22 aa, synthetic construct
  REGION 1..22: note = Equine IL-13 cyclic epitope
CVIVLVKDLLKNLRKIFHGGKC

SEQ ID NO: 137 - protein, 20 aa, synthetic construct
  REGION 1..20: note = Equine IL-13 signal peptide
MALWLTAVIALACLGGLASP

SEQ ID NO: 138 - RNA, 60 nt, synthetic construct
  misc_feature 1..60: note = Equine IL-13 signal peptide mRNA
  source 1..60: mol_type = other RNA
atggccctgtggctgaccgccgtgatcgccctggcctgcctgggcggcctggccagcccc

SEQ ID NO: 139 - protein, 5 aa, synthetic construct
  REGION 1..5: note = Spacer
GGGGS

SEQ ID NO: 140 - protein, 20 aa, synthetic construct
  REGION 1..20: note = Murine IL-4 signal peptide
MGLNPQLVVILLFFLECTRS

SEQ ID NO: 141 - RNA, 60 nt, synthetic construct
  misc_feature 1..60: note = Murine IL-4 signal peptide mRNA
  source 1..60: mol_type = other RNA
atgggcctgaacccccagctggtggtgatcctgctgttcttcctggagtgcacccgcagc

SEQ ID NO: 142 - protein, 18 aa, synthetic construct
  REGION 1..18: note = Murine IL-13 signal peptide
MALWVTAVLALACLGGLA

SEQ ID NO: 143 - RNA, 54 nt, synthetic construct
  misc_feature 1..54: note = Murine IL-13 signal peptide mRNA
  source 1..54: mol_type = other RNA
atggccctgtgggtgaccgccgtgctggccctggcctgcctgggcggcctggcc

SEQ ID NO: 144 - RNA, 525 nt, synthetic construct
  source 1..525: mol_type = other RNA
atgggcctgaacccccagctggtggtgatcctgctgttcttcctggagtgcaccaggagccacatccacggctgcgacaagaaccacctgagggagatcatcggcatcctgaacgaggtgaccggcgagggcaccccctgcaccgagatggacgtgcccaacgtgctgaccgccaccaagaacaccaccgagagcgagctggtgtgcagggccagcaaggtgctgaggatcttctacctgaagcacggcaagaccccctgcctgaagaagaacagcagcgtgctgatggagctgcagaggctgttcagggccttcaggtgcctggacagcagcatcagctgcaccatgaacgagagcaagagcaccagcctgaaggacttcctggagagcctgaagagcatcatgcagatggactacagcggccccatcctgatgcagtacatcaaggccaacagcaagttcatcggcatccccatgggcctgccccagagcatcgccctgagcagcctgatggtggcccagtag

SEQ ID NO: 145 - RNA, 1479 nt, synthetic construct
  source 1..1479: mol_type = other RNA
atgggcctgaacccccagctggtggtgatcctgctgttcttcctggagtgcaccaggagccacatccacggctgcgacaagaaccacctgagggagatcatcggcatcctgaacgaggtgaccggcgagggcaccccctgcaccgagatggacgtgcccaacgtgctgaccgccaccaagaacaccaccgagagcgagctggtgtgcagggccagcaaggtgctgaggatcttctacctgaagcacggcaagaccccctgcctgaagaagaacagcagcgtgctgatggagctgcagaggctgttcagggccttcaggtgcctggacagcagcatcagctgcaccatgaacgagagcaagagcaccagcctgaaggacttcctggagagcctgaagagcatcatgcagatggactacagcggccccgtgagcatcgacaagttcaggatcttctgcaaggccaaccccaagaagggcctgaagttcatcatcaagaggtacacccccaacaacgagatcgacagcaagggcatcagggaggacaacaacatcaccctgaagctggacaggtgcaacaacaagggcgaaaagaagatcgccaagatggagaaggccagcagcgtgttcaacgtggtgaacaagggcttcaacaacttcaccgtgagcttctggctgagggtgcccaaggtgagcgccagccacctggagaagggccagtacatcaaggccaacagcaagttcatcggcatcaccgaaaagggcccccaccacaccgccctgaggcaggccatcctgtgctggggcgagctgatgaccctggccaagggccccaagtacgtgaagcagaacaccctgaagctggccaccaagggcttcttcctgctgaccaggatcctgaccatcccccagagcctggacaagggctacagcggccccctgaaggccgagatcgcccagaggctggaggacgtgaagggcgtgagcatcgacaagttcaggatcttctgcaaggccaaccccaagaagggcctgaagttcatcatcaagaggtacacccccaacaacgagatcgacagcaagggcatcagggaggacaacaacatcaccctgaagctggacaggtgcaacaacaagggcgaaaagaagatcgccaagatggagaaggccagcagcgtgttcaacgtggtgaacaagggcttcaacaacttcaccgtgagcttctggctgagggtgcccaaggtgagcgccagccacctggagaagggccagtacatcaaggccaacagcaagttcatcggcatcaccgaaaagggcccccaccacaccgccctgaggcaggccatcctgtgctggggcgagctgatgaccctggccaagggccccaagtacgtgaagcagaacaccctgaagctggccaccaagggcttcttcctgctgaccaggatcctgaccatcccccagagcctggacaagggctag

SEQ ID NO: 146 - RNA, 2034 nt, synthetic construct
  source 1..2034: mol_type = other RNA
atgggcctgaacccccagctggtggtgatcctgctgttcttcctggagtgcaccaggagccacatccacggctgcgacaagaaccacctgagggagatcatcggcatcctgaacgaggtgaccggcgagggcaccccctgcaccgagatggacgtgcccaacgtgctgaccgccaccaagaacaccaccgagagcgagctggtgtgcagggccagcaaggtgctgaggatcttctacctgaagcacggcaagaccccctgcctgaagaagaacagcagcgtgctgatggagctgcagaggctgttcagggccttcaggtgcctggacagcagcatcagctgcaccatgaacgagagcaagagcaccagcctgaaggacttcctggagagcctgaagagcatcatgcagatggactacagcggccccggcgccgacgacgtggtggacagcagcaagagcttcgtgatggagaacttcagcagctaccacggcaccaagcccggctacgtggacagcatccagaagggcatccagaagcccaagagcggcacccagggcaactacgacgacgactggaaggagttctacagcaccgacaacaagtacgacgccgccggctacagcgtggacaacgagaaccccctgagcggcaaggccggcggcgtggtgaaggtgacctaccccggcctgaccaaggtgctggccctgaaggtggacaacgccgagaccatcaagaaggagctgggcctgagcctgaccgagcccctgatggagcaggtgggcaccgaggagttcatcaagaggttcggcgacggcgccagcagggtggtgctgagcctgcccttcgccgagggcagcagcagcgtggagtacatcaacaactgggagcaggccaaggccctgagcgtggagctggagatcaacttcgagaccaggggcaagaggggccaggacgccatgtacgagtacatggcccaggcctgcgccggcaacagggtgaggaggagcgtgggcagcagcctgagctgcatcaacctggactgggacgtgatcagggacaagaccaagaccaagatcgagagcctgaaggagcacggccccatcaagaacaagatgagcgagagccccaacaagaccgtgagcgaggagaaggccaagcagtacctggaggagttccaccagaccgccctggagcaccccgagctgagcgagctgaagaccgtgaccggcaccaaccccgtgttcgccggcgccaactacgccgcctgggccgtgaacgtggcccaggtgatcgacagcgagaccgccgacaacctggagaagaccaccgccgccctgagcatcctgcccggcatcggcagcgtgatgggcatcgccgacggcgccgtgcaccacaacaccgaggagatcgtggcccagagcatcgccctgagcagcctgatggtggcccaggccatccccctggtgggcgagctggtggacatcggcttcgccgcctacaacttcgtggagagcatcatcaacctgttccaggtggtgcacaacagctacaacaggcccgcctacagccccggccacaagacccagcccttcctgcacgacggctacgccgtgagctggaacaccgtggaggacagcatcatcaggaccggcttccagggcgagagcggccacgacatcaagatcaccgccgagaacacccccctgcccatcgccggcgtgctgctgcccaccatccccggcaagctggacgtgaacaagagcaagacccacatcagcgtgaacggcaggaagatcaggatgaggtgcagggccatcgacggcgacgtgaccttctgcaggcccaagagccccgtgtacgtgggcaacggcgtgcacgccaacctgcacgtggccttccacaggagcagcagcgaaaagatccacagcaacgagatcagcagcgacagcatcggcgtgctgggctaccagaagaccgtggaccacaccaaggtgaacagcaagctgagcctgttcttcgagatcaagagctag

SEQ ID NO: 147 - RNA, 423 nt, synthetic construct
  source 1..423: mol_type = other RNA
atgggcctgaacccccagctggtggtgatcctgctgttcttcctggagtgcaccaggagccacatccacggctgcgacaagaaccacctgagggagatcatcggcatcctgaacgaggtgaccggcgagggcaccccctgcaccgagatggacgtgcccaacgtgctgaccgccaccaagaacaccaccgagagcgagctggtgtgcagggccagcaaggtgctgaggatcttctacctgaagcacggcaagaccccctgcctgaagaagaacagcagcgtgctgatggagctgcagaggctgttcagggccttcaggtgcctggacagcagcatcagctgcaccatgaacgagagcaagagcaccagcctgaaggacttcctggagagcctgaagagcatcatgcagatggactacagctag

SEQ ID NO: 148 - RNA, 1668 nt, synthetic construct
  source 1..1668: mol_type = other RNA
atgggcctgaacccccagctggtggtgatcctgctgttcttcctggagtgcaccaggagcggcgccgacgacgtggtggacagcagcaagagcttcgtgatggagaacttcagcagctaccacggcaccaagcccggctacgtggacagcatccagaagggcatccagaagcccaagagcggcacccagggcaactacgacgacgactggaaggagttctacagcaccgacaacaagtacgacgccgccggctacagcgtggacaacgagaaccccctgagcggcaaggccggcggcgtggtgaaggtgacctaccccggcctgaccaaggtgctggccctgaaggtggacaacgccgagaccatcaagaaggagctgggcctgagcctgaccgagcccctgatggagcaggtgggcaccgaggagttcatcaagaggttcggcgacggcgccagcagggtggtgctgagcctgcccttcgccgagggcagcagcagcgtggagtacatcaacaactgggagcaggccaaggccctgagcgtggagctggagatcaacttcgagaccaggggcaagaggggccaggacgccatgtacgagtacatggcccaggcctgcgccggcaacagggtgaggaggagcgtgggcagcagcctgagctgcatcaacctggactgggacgtgatcagggacaagaccaagaccaagatcgagagcctgaaggagcacggccccatcaagaacaagatgagcgagagccccaacaagaccgtgagcgaggagaaggccaagcagtacctggaggagttccaccagaccgccctggagcaccccgagctgagcgagctgaagaccgtgaccggcaccaaccccgtgttcgccggcgccaactacgccgcctgggccgtgaacgtggcccaggtgatcgacagcgagaccgccgacaacctggagaagaccaccgccgccctgagcatcctgcccggcatcggcagcgtgatgggcatcgccgacggcgccgtgcaccacaacaccgaggagatcgtggcccagagcatcgccctgagcagcctgatggtggcccaggccatccccctggtgggcgagctggtggacatcggcttcgccgcctacaacttcgtggagagcatcatcaacctgttccaggtggtgcacaacagctacaacaggcccgcctacagccccggccacaagacccagcccttcctgcacgacggctacgccgtgagctggaacaccgtggaggacagcatcatcaggaccggcttccagggcgagagcggccacgacatcaagatcaccgccgagaacacccccctgcccatcgccggcgtgctgctgcccaccatccccggcaagctggacgtgaacaagagcaagacccacatcagcgtgaacggcaggaagatcaggatgaggtgcagggccatcgacggcgacgtgaccttctgcaggcccaagagccccgtgtacgtgggcaacggcgtgcacgccaacctgcacgtggccttccacaggagcagcagcgaaaagatccacagcaacgagatcagcagcgacagcatcggcgtgctgggctaccagaagaccgtggaccacaccaaggtgaacagcaagctgagcctgttcttcgagatcaagagctag

(19) United States
(12) Patent Application Publication
(10) Pub. No.: US 2025/0312427 A1
(43) Pub. Date: Oct. 9, 2025

(54) mRNA VACCINES COMPRISING IL-4 AND/OR IL-13 RNA AND USES THEREOF

(71) Applicant: NEOVACS, Paris (FR)

(72) Inventors: Beatrice DROUET, Paris (FR); Olivier DHELLIN, Paris (FR); Bernard FANGET, Paris (FR); Florian GAUTHIER, Paris (FR); Vincent SERRA, Paris (FR)

(73) Assignee: NEOVACS, Paris (FR)

(21) Appl. No.: 18/685,004

(22) PCT Filed: Aug. 19, 2022
(86) PCT No.: PCT/EP2022/073222

§ 371 Date: Feb. 20, 2024

Related U.S. Application Data
(60) Provisional application No. 63/235,351, filed on Aug. 20, 2021.

(30) Foreign Application Priority Data
Aug. 20, 2021 (EP) 21306131.0

(51) Int. Cl. A61K 39/00; A61K 39/385; A61P 37/08; C07K 14/54

(52) U.S. Cl. CPC A61K 39/0008; A61K 39/385; A61P 37/08; C07K 14/5406; C07K 14/5437; A61K 2039/5258; A61K 2039/53; A61K 2039/6037

(57) ABSTRACT

An mRNA vaccine including at least one RNA molecule that encodes at least one cytokine (preferably IL-4, IL-13 or fragments thereof) and at least one T cell epitope, for treating or preventing disorders associated with aberrant IL-4 and/or IL-13 expression or activity, in particular asthma, atopic dermatitis and allergic disorders.

### FIELD OF INVENTION

The present invention relates to a mRNA vaccine and to the use thereof for treating disorders associated with aberrant IL-4 and/or IL-13 expression or activity, in particular asthma, atopic dermatitis and allergic disorders.

### BACKGROUND OF INVENTION

Allergic disorders are complex diseases resulting from interactions between multiple genetic and environmental factors. The increase in allergies observed in the past decades is explained mostly by environmental changes occurring in the same period. Among all allergies, allergic asthma, allergic rhinitis and food allergies are major public health problems. It is estimated that half of the global population will be affected by an allergic disease by 2050. Thus, the increase in allergic diseases has become an important health issue throughout the globe resulting in significant socioeconomic burden and for which there is still no efficient long-term therapy.

Pathogenesis of allergic disorders results from the exposure of the immune system to allergens. Such exposures are considered to be responsible of a breakdown of tolerance, resulting in type 2 immune responses characterized by the production of T helper cell type 2 (Th2) cytokines such as interleukin 4 (IL-4) and interleukin 13 (IL-13), high levels of immunoglobulin E (IgE) antibodies, and infiltration and expansion of immune cells within the inflamed tissue. Mast cells, basophils, and eosinophils are especially involved in the release of cytoplasmic granules containing preformed inflammatory mediators such as histamine.

IL-4 and IL-13 cytokines thus play key roles in the pathogenesis of allergic disorders. Both cytokines have long been associated with the pathogenesis of allergic disorders and are therapeutically important cytokines based on their biological functions. IL-4 and IL-13 present similar structure and share one receptor subunit (IL-4Rα). However, despite their many similarities, IL-4 and IL-13 are thought to play non-redundant functions in allergy.

IL-4 is a pleiotropic cytokine involved in the development of allergy (Gour N. & Wills-Karp M., 2015), as increased IL-4 levels have been observed in serum and in bronchoalveolar lavage of asthmatic patients. IL-4 is considered to specifically act in the early phase of allergy development. The crucial role of IL-4 lies in its multiple effects which drive to allergy such as, for example, induction of IgE production, up-regulation of IgE receptor expression and differentiation of naïve T helper cell type 0 (Th0) into Th2 lymphocytes.

In contrast, IL-13 is more involved in effector and late phases of allergic reactions (Gour N. & Wills-Karp M., 2015). It has been shown that IL-13 is sufficient to induce the main manifestations of allergic diseases including, without limitation, airways hyperresponsiveness, mucus production, airway smooth muscle alterations and sub-epithelial fibrosis.

Therefore, IL-4 and IL-13 are promising therapeutic targets for the treatment of allergies, and there is a clear need to improve current strategies to block these molecules, in particular in order to reach long-term therapeutic effects.

Recently, novel therapies have been developed to treat or prevent allergies. These treatments, based on passive immunization, specifically target pathogenic factors involved in allergy. For example, the use of recombinant antibodies directed to IL-4 and IL-13 or their receptors was described in the art. However, use of recombinant antibodies is limited by high cost, the need to perform repeated injections, and potential risks of appearance of anti-drug antibodies (ADAs) or other adverse reactions.

RNA vaccines have emerged as a new therapeutical perspective in 1990. Huge advantages may be associated with the use of RNA as vaccine in comparison to other types of vaccines (e.g., attenuated virus and DNA-based vaccine). Indeed, using RNA vaccine may be safer due to the absence of risks of mutagenesis or infection. Furthermore, the immunogenicity of RNA can be modulated prior to in vivo delivery. A higher efficiency of the vaccination process may also be obtained via in vitro modifications of RNA, thereby increasing its stability and translatability. Finally, RNA vaccines may be produced very quickly and at low cost.

However, many difficulties need to be overcome for using RNA as a vaccine, in particular RNA instability, high innate immunogenicity and in vivo delivery of the RNA.

In the present invention, the Applicant provides an RNA vaccine that may be used to induce the production of antibodies against self-proteins (i.e., IL-4 and IL-13), whereas usual mRNA vaccines induce the production by the receiving subject of antibodies against nonself-proteins. In particular, the RNA vaccines disclosed in the present invention efficiently induce production of antibodies against self-proteins in the receiving subject, without substantial toxicity.

The Applicant thus herein provides a novel RNA vaccine comprising RNA sequences encoding a cytokine selected from IL-4 and IL-13 (or fragments thereof) and T cell epitopes. This novel mRNA vaccine is of particular interest for treating inflammatory disorders, such as, in particular asthma, atopic dermatitis and allergic disorders.

### SUMMARY

The present invention relates to a composition comprising at least one RNA molecule, wherein the at least one RNA molecule encodes at least one amino acid sequence comprising:
at least one cytokine, or at least one fragment or epitope thereof, preferably wherein the at least one cytokine is interleukin-4 (IL-4) and/or interleukin-13 (IL-13),
at least one T cell epitope, and
optionally at least one spacer.

In one embodiment, the at least one cytokine is IL-4.

In one embodiment, the IL-4 fragment is selected from the group consisting of SEQ ID NOs: 7-10, 13-16, 94-97 and 109-112.

In one embodiment, the at least one cytokine is IL-13.

In one embodiment, the IL-13 fragment is selected from the group comprising or consisting of SEQ ID NOs: 25-28, 35-38, 119-122 and 129-132.

In one embodiment, the at least one RNA molecule encodes IL-4 or at least one fragment or epitope thereof and IL-13 or at least one fragment or epitope thereof.

In one embodiment, the at least one T cell epitope is selected from the group comprising or consisting of CRM₁₉₇, combination of diphteria and tetanus epitopes (TpD), epitopes of tetanus toxin (TT), universal CD4 polyepitopes, variants and fragments thereof.

In one embodiment, the at least one spacer is selected from the group comprising or consisting of PMGLP, cathepsin cleavage sites, amino acids doublets, GP, GPGPG, GGSGGGGSGG, (GGGGS)ₙ wherein n ranges from 1 to 4, LG, ASG, KG and RR.

In one embodiment, the at least one RNA molecule is encapsulated, preferably in a nanoparticle (e.g., a lipid nanoparticle), in a liposome or in a virus-like particle.

The present invention further relates to a pharmaceutical composition comprising the composition as described herein and at least one pharmaceutically acceptable excipient.

Another object of the present invention is a vaccine composition comprising the composition as described herein and optionally at least one adjuvant.

Another object of the present invention is a composition as described herein for use as a medicament.

The present invention further relates to a composition, a pharmaceutical composition or a vaccine composition as described herein, for use in treating an inflammatory disorder, preferably wherein said disorder is associated with aberrant IL-4 and/or IL-13 expression or activity.

In one embodiment, the inflammatory disorder is selected from the group comprising or consisting of asthma (either allergic or non-allergic), allergic conditions (such as, for example, food allergies, venom allergy, allergy to animals, drug allergy, hyper IgE syndrome, allergic rhinitis, allergic conjunctivitis and allergic enterogastritis), atopic disorders (such as, for example, atopic dermatitis, urticaria (including chronic idiopathic urticaria and chronic spontaneous urticaria), eczema), bullous pemphigoid, respiratory disorders (such as allergic and nonallergic asthma, chronic obstructive pulmonary disease (COPD)), nasal polyposis and other conditions involving airway inflammation (such as, for example, eosinophilia, fibrosis and excess mucus production including cystic fibrosis and pulmonary fibrosis, systemic sclerosis (SSc)); inflammatory and/or autoimmune disorders or conditions, gastrointestinal disorders or conditions (such as, for example, inflammatory bowel diseases (IBD) and eosinophilic esophagitis (EE), and eosinophilic-mediated gastrointestinal disease, ulcerative colitis and Crohn's disease); systemic lupus erythematosus, liver disorders or conditions (such as, for example, cirrhosis, and hepatocellular carcinoma), scleroderma; fibrotic diseases or disorders (such as, for example, fibrosis of the liver (such as, for example, fibrosis caused by a hepatitis B and/or C virus)), scleroderma; solid tumors or cancers such as leukemia (such as, for example, B cell chronic lymphocytic leukaemia), glioblastoma, lymphoma (such as, for example, Hodgkin's lymphoma) and mastocytosis. In one embodiment, the inflammatory disorder is selected from the group comprising or consisting of asthma (either allergic or non-allergic), allergic conditions (such as, for example, food allergies, venom allergy, allergy to animals, drug allergy, anaphylaxis, hyper IgE syndrome, allergic rhinitis, allergic conjunctivitis and allergic enterogastritis), atopic disorders (such as, for example, atopic dermatitis, urticaria (including chronic idiopathic urticaria and chronic spontaneous urticaria), eczema), bullous pemphigoid, respiratory disorders (such as allergic and nonallergic asthma, chronic obstructive pulmonary disease (COPD)), nasal polyposis and other conditions involving airway inflammation (such as, for example, eosinophilia, fibrosis and excess mucus production including cystic fibrosis and pulmonary fibrosis, systemic sclerosis (SSc)); inflammatory and/or autoimmune disorders or conditions, gastrointestinal disorders or conditions (such as, for example, inflammatory bowel diseases (IBD) and eosinophilic esophagitis (EE), and eosinophilic-mediated gastrointestinal disease, ulcerative colitis and Crohn's disease); systemic lupus erythematosus, liver disorders or conditions (such as, for example, cirrhosis, and hepatocellular carcinoma), scleroderma; fibrotic diseases or disorders (such as, for example, fibrosis of the liver (such as, for example, fibrosis caused by a hepatitis B and/or C virus)), scleroderma; solid tumors or cancers such as leukemia (such as, for example, B cell chronic lymphocytic leukaemia), glioblastoma, lymphoma (such as, for example, Hodgkin's lymphoma) and mastocytosis.

In one embodiment, the inflammatory disorder is selected from the group comprising or consisting of asthma (e.g., allergic asthma), atopic dermatitis, chronic obstructive pulmonary disease (COPD), pulmonary fibrosis, food allergy, nasal polyposis and eosinophilic esophagitis, preferably said inflammatory disorder is allergy, asthma, or atopic dermatitis.

### Definitions

In the present invention, the following terms have the following meanings:

As used herein, the term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

As used herein, an “adjuvant” is a substance that enhances the immunogenicity of a composition of this invention. Adjuvants are often given to boost the immune response and are well known to the skilled artisan.

As used herein, the term “antigen presenting cell” or “APC” refers to an immune system cell such as an accessory cell (e.g., a B cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

As used herein, the term “derived from” indicates a relationship between a first and a second molecule. It generally refers to structural similarity between the first molecule and a second molecule and does not connote or include a process or source limitation on a first molecule that is derived from a second molecule.

As used herein, the term “epitope” refers to a specific arrangement of amino acids located on a protein to which an antibody or an MHC molecule or any binding fragment thereof binds. Epitopes may consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the protein that may not necessarily be contiguous.

In particular, as used herein, the term “T cell epitope” refers to an epitope that may be recognized and bound by a TCR (expressed in particular by a CD4⁺ T cell (e.g., a Th2 cell)) when presented by a type II MHC molecule. In one embodiment, the T cell expressing the TCR is a T helper cell (e.g., a Th2 cell), and the binding of the T cell epitope to the TCR induces the activation of the T cell, thereby leading to the production of proinflammatory molecules by said T cell.

As used herein, the term “B cell epitope” refers to an epitope that may be recognized and bound by a B cell receptor (BCR) expressed by a B cell, when present as soluble molecule in said B cell microenvironment, or cross-linked through the BCR when presented at the surface of various cell types. In one embodiment, binding of the B cell epitope to the BCR induces the activation of the B cell, thereby leading to its activation and production of specific antibodies by said B cell.

As used herein, the term “immune response” refers to a reaction occurring within an organism, in particular in response to foreign elements. It may thus refer to the action, for example of lymphocytes (such as B cells and T cells, including CD4+, CD8+, Th1 and Th2 cells), antigen presenting cells (such as, for example, professional antigen presenting cells such as dendritic cells), natural killer cells, myeloid cells (such as, for example, macrophages, eosinophils, mast cells, basophils, and granulocytes), and macromolecules produced by the above cells or the liver (including, without limitation, antibodies, cytokines and complement). The term “immune response”, as used herein, thus includes T cell-mediated and/or B cell-mediated immune responses.

As used herein, an antibody that “inhibits the biological activity” or “neutralizes the biological activity” of at least one cytokine selected from IL-4, IL-13 or mixtures thereof is intended to refer to an antibody that inhibits the activity of that cytokine by at least 10%, 20%, 30%, 40%, 50%, 60%, 70%, or 80% or more, as compared to the level of activity of the cytokine in the absence of the antibody. Examples of functional assays that may be used for assessing the activity of a cytokine are well known in the art.

As used herein, the term “pharmaceutically acceptable excipient” refers to an excipient that does not produce an adverse, allergic or other untoward reaction when administered to an animal, in particular a mammal, preferably a human. It includes any and all solvents, dispersion media, coatings, antibacterial and antifungal agents, isotonic and absorption delaying agents and the like. A pharmaceutically acceptable excipient may thus refer to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA or EMA.

As used herein, the term “subject” is intended to include living organisms in which an immune response can be elicited (e.g., mammals, in particular human, primates, dogs, cats, horses, sheep and the like). In one embodiment, the subject is a human. In one embodiment, a subject may be a “patient”, i.e., a warm-blooded animal, preferably a human, who/which is awaiting the receipt of, or is receiving medical care or was/is/will be the object of a medical procedure or is monitored for the development of the targeted disease or condition, such as, for example, an inflammatory disorder. In one embodiment, the subject is an adult (for example a subject above the age of 18). In another embodiment, the subject is a child (for example a subject below the age of 18). In one embodiment, the subject is a male. In another embodiment, the subject is a female. In one embodiment, the subject is affected, preferably is diagnosed, with an inflammatory disorder. In one embodiment, the subject is at risk of developing an inflammatory disorder. Examples of risks factor include, but are not limited to, genetic predisposition, or familial history of inflammatory disorders.

As used herein, the terms “therapeutically effective amount” refers to an amount of the composition as described herein, effective to achieve a particular biological result. Thus, the terms “therapeutically effective amount” mean a level or amount of a composition that is aimed at, without causing significant negative or adverse side effects to the target, (1) delaying or preventing the onset of the targeted disease or condition; (2) slowing down or stopping the progression, aggravation, or deterioration of one or more symptoms of the targeted disease or condition; (3) bringing about ameliorations of the symptoms of the targeted disease or condition; (4) reducing the severity or incidence of the targeted disease or condition; or (5) curing the targeted disease or condition. A therapeutically effective amount may be administered prior to the onset of the targeted disease or condition, for a prophylactic or preventive action. Alternatively, or additionally, the therapeutically effective amount may be administered after initiation of the targeted disease or condition, for a therapeutic action.

As used herein, the term “treatment” or “treating” refers to both therapeutic treatment and prophylactic or preventative measures; wherein the object is to prevent or slow down (lessen) the targeted disease or condition. Those in need of treatment include those already with the condition as well as those prone to have the condition or those in whom the condition is to be prevented. A subject is successfully “treated” for a disease or condition if, after receiving a therapeutic amount of a composition as described herein, the subject shows observable and/or measurable improvement in one or more of the following: reduction in the number of pathogenic cells; reduction in the percent of total cells that are pathogenic; relief to some extent of one or more of the symptoms associated with the specific condition; reduced morbidity and mortality, and/or improvement in quality of life issues. The above parameters for assessing successful treatment and improvement in the condition are readily measurable by routine procedures familiar to a physician.

### DETAILED DESCRIPTION

The present invention relates to at least one RNA molecule, or to a composition comprising said at least one RNA molecule, wherein the at least one RNA molecule encodes (i) an amino acid sequence of at least one cytokine or cytokine fragment, (ii) an amino acid sequence of at least one T cell epitope and optionally (iii) an amino acid sequence of at least one spacer.

In one embodiment, the at least one RNA molecule encodes a single protein construct (preferably a single one-chain protein construct) comprising, in a single amino acid chain, (i) an amino acid sequence of at least one cytokine or cytokine fragment, (ii) an amino acid sequence of at least one T cell epitope and optionally (iii) an amino acid sequence of at least one spacer.

In one embodiment, the at least one RNA molecule encodes a single protein construct (preferably in a single one-chain protein construct) comprising, in a single amino acid chain, from N-terminal to C-terminal (i) an amino acid sequence of at least one cytokine or cytokine fragment, optionally (ii) an amino acid sequence of at least one spacer and (iii) an amino acid sequence of at least one T cell epitope. In one embodiment, the single protein construct further comprises a signal peptide, preferably in N-terminal.

In one embodiment, the RNA molecule of the present invention comprises, from 5′ to 3′, (i) a sequence encoding at least one cytokine or cytokine fragment, optionally (ii) a sequence encoding at least one spacer and (iii) a sequence encoding at least one T cell epitope. In one embodiment, the RNA molecule further comprises a sequence encoding a signal peptide, preferably in 5′.

In one embodiment, the at least one T cell epitope is not a T cell epitope from the at least one cytokine. More preferably, the at least one T cell epitope originates from another organism than the at least one cytokine.

In one embodiment, the at least one cytokine or cytokine fragment originates from the same organism than the subject to be treated. For example, when the RNA molecules are intended to be used for treating humans, the at least one cytokine or fragment thereof is human (either natural or recombinant).

Thus, in one embodiment, the at least one RNA molecule may be described as comprising two domains: a first domain encoding at least one cytokine or fragment thereof (wherein said at least one cytokine preferably originates from the same organism than the subject to be treated—said domain may thus be defined as a “self-domain”), and a second domain comprising at least one T cell epitope (wherein preferably said at least one T cell epitope originates from another organism than the subject to be treated—said domain may thus be defined as a “non-self-domain”).

In one embodiment, the at least one RNA molecule encodes at least one epitope of said at least one cytokine or fragment thereof. In one embodiment, said at least one epitope is a B cell epitope. In another embodiment, said at least one epitope is a T cell epitope.

In one embodiment, the at least one RNA molecule encodes a cytokine or fragment thereof having a reduced cytokine activity. Without willing to be bound to any theory, reduction of the cytokine activity may be of particular relevance, as induction of the expression of a cytokine with native activity may induce toxicity. Indeed, in one embodiment, the present invention aims at treating diseases associated with aberrant IL-4 and/or IL-13 activity, and administering a RNA molecule resulting in the expression of increased amounts of functional IL-4 and/or IL-13 may induce serious side effects.

In one embodiment, the residual cytokine activity of the cytokine or fragment thereof encoded by the at least one RNA molecule of the present invention is equal or lower than 80%, 70%, 60%, 50%, 40%, 30%, 20%, 10% or less of the cytokine activity measured for the cytokine from which it derives. Assays that may be used for measuring a cytokine activity are well known in the art.

In one embodiment, the cytokine is IL-4 or IL-13 and the residual cytokine activity of the cytokine or fragment thereof encoded by the at least one RNA molecule of the present invention may be measured in the condition of Test A. Test A is an in vitro test based on the measure of proliferating activity induced by serial dilutions of the supernatant of cells (e.g., HEK293 cells) transfected with the at least one RNA molecule of the present invention (preferably wherein the supernatant is collected 24 h post transfection) on CTLL-2 cells, e.g., after about 48 hours of incubation. Methods for measuring cell proliferation are well known in the part and include, without limitation, MTS/PMS assay (including addition of the substrate and reading of optical density at 490 nm after 4 h of incubation). An example of Test A is shown in Example 2.

In one embodiment, the residual cytokine activity of the cytokine or fragment thereof encoded by the at least one RNA molecule of the present invention, when measured in the conditions of Test A, is equal or lower than 80%, 70%, 60%, 50%, 40%, 30%, 20%, 10% or less of the cytokine activity measured for the cytokine from which it derives, at the same dilution.

Without willing to be bound to any theory, the Applicant suggests that the at least one RNA molecule, after production in vitro and delivery in vivo to targeted cells (such as, for example, antigen presenting cells (APC), in particular dendritic cells) leads to the production in these cells of at least one protein or peptide. The peptide(s) or protein(s) may then either be secreted by the cell in the extracellular compartment or degraded by the proteasome and processed into the endoplasmic reticulum in order to generate epitopes which are afterwards presented onto the cell membrane by the MHC class I and class II molecules.

The presentation of IL-4 and/or IL-13 epitopes (from the RNA constructs of the invention) via the MHC class II molecule may lead to the activation of CD4+ T cells which in turn will activate B lymphocytes, thereby leading to the production of antibodies specific said epitope(s). In addition, the IL-4 and/or IL-13 epitopes may also be presented by MHC class I molecule and induce the activation of cytotoxic antigen-specific CD8+ T cells, that may then lysate cells expressing the same antigen at their cell surface. In addition, the presentation of T cell epitopes of the second domain of the RNA molecule of the invention by MHC molecules may increase the immune response induced following presentation of IL-4 and/or IL-13 epitopes.

In one embodiment, the at least one RNA molecule is an mRNA molecule, preferably a non-replicating mRNA molecule encoding at least one cytokine or fragment thereof, at least one T cell epitope and optionally at least one spacer.

In one embodiment, the at least one cytokine or fragment thereof, the at least one T cell epitope, and optionally the at least one spacer are encoded by different mRNA molecules. In one embodiment, the at least one cytokine or fragment thereof, the at least one T cell epitope, and optionally the at least one spacer are encoded by a single mRNA molecule. In one embodiment, the at least one cytokine or fragment thereof, the at least one T cell epitope, and optionally the at least one spacer are comprised in a single protein construct, preferably in a single one-chain protein construct.

In one embodiment, the at least one cytokine is IL-4.

In one embodiment, IL-4 is derived from a mammalian IL-4.

In one embodiment, IL-4 is a variant of a mammal IL-4, wherein said variant presents at least about 70%, 75%, 80%, 85%, 90%, 95% or more identity with the mammal IL-4 from which it derives.

As used herein, the term “identity” or “identical”, when used in a relationship between the sequences of two or more nucleic acid sequences or of two or more polypeptides, refers to the degree of sequence relatedness between nucleic acid sequences or polypeptides, as determined by the number of matches between strings of two or more nucleic or amino acid residues, respectively. “Identity” measures the percent of identical matches between the smaller of two or more sequences with gap alignments (if any) addressed by a particular mathematical model or computer program (i.e., “algorithms”). Identity of related nucleic acid sequences or polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988). Preferred methods for determining identity are designed to give the largest match between the sequences tested. Methods of determining identity are described in publicly available computer programs. Preferred computer program methods for determining identity between two sequences include ClustalO (Sievers F., et al 2011), the GCG program package, including, GAP (Devereux et al., Nucl. Acid. Res. 2, 387 (1984); Genetics Computer Group, University of Wisconsin, Madison, Wis.), BLASTP, BLASTN, and FASTA (Altschul et al., J. MoI. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

In one embodiment, the at least one cytokine is a fragment of IL-4, such as, for example, a fragment of IL-4 comprising at least about 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120 or 125 amino acids (preferably contiguous amino acids) of the IL-4 molecule from which it derives.

In one embodiment, the fragments of IL-4 may comprise or consist of epitopes. In one embodiment, said fragment of IL-4 comprises or consists of at least one epitope of IL-4. Examples of epitopes of IL-4 are listed below.

In one embodiment, IL-4 is full-length IL-4.

In one embodiment of the present invention, IL-4 is human IL-4. Human IL-4 has a sequence SEQ ID NO: 1 (UniProt ID: P05112-1).

SEQ ID NO: 1
HKCDITLQEIIKTLNSLTEQKTLCTELTVTDIFAASKNTTEKETFCRAAT
VLRQFYSHHEKDTRCLGATAQQFHRHKQLIRFLKRLDRNLWGLAGLNSCP
VKEANQSTLENFLERLKTIMREKYSKCSS

In one embodiment, the RNA sequence encoding the at least one cytokine comprises or consists of a sequence encoding human IL-4. An example of RNA sequence encoding human IL-4 comprises or consists of SEQ ID NO: 2, or a sequence having at least about 70%, preferably at least about 75%, 80%, 85%, 90%, 95% or more identity with SEQ ID NO: 2.

SEQ ID NO: 2
CACAAGUGCGACAUCACCCUGCAGGAGAUCAUCAAGACCCUGAACAGCCU
GACCGAGCAGAAGACCCUGUGCACCGAGCUGACCGUGACCGACAUCUUCG
CCGCCAGCAAGAACACCACCGAGAAGGAGACCUUCUGCCGCGCCGCCACC
GUGCUGCGCCAGUUCUACAGCCACCACGAGAAGGACACCCGCUGCCUGGG
CGCCACCGCCCAGCAGUUCCACCGCCACAAGCAGCUGAUCCGCUUCCUGA
AGCGCCUGGACCGCAACCUGUGGGGCCUGGCCGGCCUGAACAGCUGCCCC
GUGAAGGAGGCCAACCAGAGCACCCUGGAGAACUUCCUGGAGCGCCUGAA
GACCAUCAUGCGCGAGAAGUACAGCAAGUGCAGCAGC

In one embodiment, the at least one IL-4 cytokine fragment is a peptide comprising or consisting of at least one epitope of human IL-4.

Examples of epitopes of human IL-4 include, but are not limited to:

```
  (SEQ ID NO: 3)
LQEIIKTLNSLTEQKTLCTELT,
  (SEQ ID NO: 4)
KETFCRAATVLRQFY,
(SEQ ID NO: 5)
AQQFHRHKQLIRFLKRLDRNLWGLAG
and
(SEQ ID NO: 6)
LENFLERLKTIMREKYSKC
```

Thus, according to one embodiment, the at least one RNA molecule comprises one or more RNA sequences encoding a peptide comprising or consisting of at least one epitope of human IL-4, such as, for example, SEQ ID NO: 3, SEQ ID NO: 4, SEQ ID NO: 5 or SEQ ID NO: 6.

In one embodiment, one or more cysteine residue(s) are added to the extremities of peptides comprising at least one epitope of human IL-4. In one embodiment, one cysteine residue is added to each extremity of a peptide comprising or consisting of at least one epitope of human IL-4, thereby conferring a cyclic conformation to said peptide.

Examples of cyclic peptides comprising at least one epitope of human IL-4 include, but are not limited to, CLQEIIKTLNSLTEQKTLCTELTC (SEQ ID NO: 7), CKETFCRAATVLRQFYC (SEQ ID NO: 8), CAQQFHRHKQLIRFLKRLDRNLWGLAGC (SEQ ID NO: 9) and CLENFLERLKTIMREKYSKC (SEQ ID NO: 10).

Thus, according to one embodiment, the at least one RNA molecule comprises one or more RNA sequences encoding a cyclic peptide comprising or consisting of at least one epitope of human IL-4, such as, for example, SEQ ID NO: 7, SEQ ID NO: 8, SEQ ID NO: 9 or SEQ ID NO: 10.

In one embodiment, the at least one RNA sequence, in particular the at least one RNA sequence encoding human IL-4 or at least one fragment thereof further comprises a RNA sequence encoding a signal peptide, such as, for example the signal peptide of the native human IL-4 protein.

In one embodiment, the signal peptide is the signal peptide of human IL-4, preferably comprising or consisting of the sequence SEQ ID NO: 103 (MGLTSQLLPPLFFLLACAGNFVHG), that may be encoded by the RNA sequence SEQ ID NO: 104 (AUGGGCCUGACCAGCCAGCUGCUGCCCCCCCUGUU CUUCCUGCUGGCCUGCGCCGGCAACUUCGUGCACGGC).

Other examples of signal peptides that may be used in the present invention include, but are not limited to peptides having the following sequences:

(SEQ ID NO: 88, derived from Serum albumin preproprotein)
MKWVTFISLLFLESSAYS
(SEQ ID NO: 89, derived from Human IL-2)
MQLLSCIALILALV
(SEQ ID NO: 90, derived from Gaussia luciferase):
MGVKVLFALICIAVAEA
(SEQ ID NO: 91, derived from Human chymotrypsinogen)
MAFLWLLSCWALLGTTFG;
and
(SEQ ID NO: 92)
MWWRLWWLLLLLLLLWPMVWA
(SEQ ID NO: 93, derived from Human trypsinogen-2)
MNLLLILTFVAAAVA

Other examples of signal peptides that may be used in the present invention include, but are not limited to, SEQ ID NOs: 78, 84, 117, 127, 137, 140 and 142.

In one embodiment of the present invention, IL-4 is murine IL-4. Murine IL-4 has a sequence SEQ ID NO: 11 (UniProt ID: P07750-1).

SEQ ID NO: 11
HIHGCDKNHLREIIGILNEVTGEGTPCTEMDVPNVLTATKNTTESELVC
RASKVLRIFYLKHGKTPCLKKNSSVLMELQRLFRAFRCLDSSISCTMNE
SKSTSLKDFLESLKSIMQMDYS

In one embodiment, the RNA sequence encoding the at least one cytokine comprises a sequence encoding murine IL-4. An example of RNA sequence encoding murine IL-4 comprises or consists of SEQ ID NO: 12, or a sequence having at least about 70%, preferably at least about 75%, 80%, 85%, 90%, 95% or more identity with SEQ ID NO: 12.

SEQ ID: 12
CACAUCCACGGCUGCGACAAGAACCACCUGAGGGAGAUCAUCGGCAUCCU
GAACGAGGUGACCGGCGAGGGCACCCCCUGCACCGAGAUGGACGUGCCCA
ACGUGCUGACCGCCACCAAGAACACCACCGAGAGCGAGCUGGUGUGCAGG
GCCAGCAAGGUGCUGAGGAUCUUCUACCUGAAGCACGGCAAGACCCCCUG
CCUGAAGAAGAACAGCAGCGUGCUGAUGGAGCUGCAGAGGCUGUUCAGG
GCCUUCAGGUGCCUGGACAGCAGCAUCAGCUGCACCAUGAACGAGAGCAA
GAGCACCAGCCUGAAGGACUUCCUGGAGAGCCUGAAGAGCAUCAUGCAGA
UGGACUACAGC

In one embodiment, the at least one IL-4 cytokine fragment is a peptide comprising or consisting of at least one epitope of murine IL-4.

Examples of epitopes of murine IL-4 include, but are not limited to, LREIIGILNEVTGEGTPCTEMD (SEQ ID NO: 13), SELVCRASKVLRIFY (SEQ ID NO: 14), SSVLMELQRLFRAFRCLDS (SEQ ID NO: 15) and LKDFLESLKSIMQMDYS (SEQ ID NO: 16).

Thus, according to one embodiment, the at least one RNA molecule comprises one or more RNA sequences encoding a peptide comprising or consisting of at least one epitope of murine IL-4, such as, for example, SEQ ID NO: 13, SEQ ID NO: 14, SEQ ID NO: 15 or SEQ ID NO: 16.

In one embodiment, one or more cysteine residue(s) are added to the extremities of peptides comprising or consisting of at least one epitope of murine IL-4. In one embodiment, one cysteine residue is added to the extremity of a peptide comprising or consisting of at least one epitope of murine IL-4, thereby conferring a cyclic conformation to said peptide.

Examples of cyclic peptides comprising at least one epitope of murine IL-4 include but are not limited to, CLREIIGILNEVTGEGTPCTEMDC (SEQ ID NO: 17), CSELVCRASKVLRIFYC (SEQ ID NO: 18), CSSVLMELQRLFRAFRCLDSC (SEQ ID NO: 19) and CLKDFLESLKSIMQMDYSC (SEQ ID NO: 20).

Thus, according to one embodiment, the at least one RNA molecule comprises one or more RNA sequences encoding a cyclic peptide comprising or consisting of at least one epitope of murine IL-4, such as, for example, SEQ ID NO: 17, SEQ ID NO: 18, SEQ ID NO: 19 or SEQ ID NO: 20.

In one embodiment, the at least one RNA sequence, in particular the at least one RNA sequence encoding murine IL-4 or at least one fragment thereof further comprises a RNA sequence encoding a signal peptide, such as, for example the signal peptide of the native murine IL-4 protein.

In one embodiment, the signal peptide is the signal peptide of murine IL-4, preferably comprising or consisting of the sequence SEQ ID NO: 140 (MGLNPQLVVILLFFLECTRS), that may be encoded by the RNA sequence SEQ ID NO: 141 (AUGGGCCUGAACCCCCAGCUGGUGGUGAUCCUGCUGUUCUUCC UGGAGUGCACCCGCAGC).

Other examples of signal peptides that may be used in the present invention include, without limitation, SEQ ID NO: 88-93. Other examples of signal peptides that may be used in the present invention include, without limitation, SEQ ID NOs: 78, 84, 103, 117, 127, 137 and 142.

In one embodiment of the present invention, IL-4 is canine IL-4. Canine IL-4 has a sequence SEQ ID NO: 21 (UniProt ID: 077762-1).

SEQ ID NO: 21
HNFNITIKEIIKMLNILTARNDSCMELTVKDVFTAPKNTSDKEIF
CRAATVLRQIYTHNCSNRYLRGLYRNLSSMANKTCSMNEIKKSTL
KDFLERLKVIMQKKYYRH

In one embodiment, the RNA sequence encoding the at least one cytokine comprises or consists of a sequence encoding canine IL-4. An example of RNA sequence encoding canine IL-4 comprises or consists of SEQ ID NO: 22, or a sequence having at least about 70%, preferably at least about 75%, 80%, 85%, 90%, 95% or more identity with SEQ ID NO: 22.

SEQ ID NO: 22
CACAACUUCAACAUCACCAUCAAGGAGAUCAUCAAGAUGCUGAAC
AUCCUGACCGCCCGCAACGACAGCUGCAUGGAGCUGACCGUGAAG
GACGUGUUCACCGCCCCCAAGAACACCAGCGACAAGGAGAUCUUC
UGCCGCGCCGCCACCGUGCUGCGCCAGAUCUACACCCACAACUGC
AGCAACCGCUACCUGCGCGGCCUGUACCGCAACCUGAGCAGCAUG
GCCAACAAGACCUGCAGCAUGAACGAGAUCAAGAAGAGCACCCUG
AAGGACUUCCUGGAGCGCCUGAAGGUGAUCAUGCAGAAGAAGUAC
UACCGCCAC

In one embodiment, the at least one IL-4 cytokine fragment is a peptide comprising or consisting of at least one epitope of canine IL-4.

Examples of epitopes of canine IL-4 include, but are not limited to, IKEIIKMLNILTARNDSCMELT (SEQ ID NO: 94), KEIFCRAATVLRQIY (SEQ ID NO: 95), RYLRGLYRNLSSMAN (SEQ ID NO: 96) and LKDFLERLKVIMQKKY (SEQ ID NO: 97).

Thus, according to one embodiment, the at least one RNA molecule of the present invention comprises one or more RNA sequences encoding a peptide comprising or consisting of at least one epitope of canine IL-4, such as, for example, SEQ ID NO: 94-97.

In one embodiment, one or more cysteine residue(s) are added to the extremities of peptides comprising or consisting of at least one epitope of canine IL-4. In one embodiment, one cysteine residue is added to each extremity of a peptide comprising or consisting of at least one epitope of canine IL-4, thereby conferring a cyclic conformation to said peptide.

Examples of cyclic peptides comprising at least one epitope of canine IL-4 include, but are not limited to, CIKEIIKMLNILTARNDSCMELTC (SEQ ID NO: 105), CKEIFCRAATVLRQIYC (SEQ ID NO: 106), CRYLRGLYRNLSSMANC (SEQ ID NO: 107), and CLKDFLERLKVIMQKKYC (SEQ ID NO: 108).

Thus, according to one embodiment, the at least one RNA molecule comprises one or more RNA sequences encoding a cyclic peptide comprising or consisting of at least one epitope of canine IL-4, such as, for example, SEQ ID NO: 105-108.

In one embodiment, the at least one RNA sequence, in particular the at least one RNA sequence encoding canine IL-4 or at least one fragment thereof further comprises a RNA sequence encoding a signal peptide, such as, for example the signal peptide of the native canine IL-4 protein.

In one embodiment, the signal peptide is the signal peptide of canine IL-4, preferably comprising or consisting of the sequence MGLTSQLIPTLVCLLALTSTFVHG (SEQ ID NO: 78) that may be encoded by the RNA sequence AUGGGCCUGACCAGCCAGCUGAUCCCCACCCUGGUGUGC CUGCUGGCCCUGACCAGCACCUUCGUGCACGGC (SEQ ID NO: 79).

Other examples of signal peptides that may be used in the present invention include, without limitation, SEQ ID NO: 88-93. Other examples of signal peptides that may be used in the present invention include, without limitation, SEQ ID NOs: 84, 103, 117, 127, 137, 140 and 142.

In one embodiment of the present invention, IL-4 is equine IL-4. Equine IL-4 has a sequence SEQ ID NO: 99 (UniProt ID: P42202).

SEQ ID NO: 99
CKYDITLQEIIKTLNLTDGKGKNSCMELTVADAFGPKNTDGKEIC
RAAKVLQQYKRHDRSLIKECLSGLDRNLKGMANGTCCTVNEAKKS
TLKDFLERLKTIMKEKYSKCS

In one embodiment, the RNA sequence encoding the at least one cytokine comprises or consists of a sequence encoding equine IL-4. An example of RNA sequence encoding equine IL-4 comprises or consists of SEQ ID NO: 100, or a sequence having at least about 70%, preferably at least about 75%, 80%, 85%, 90%, 95% or more identity with SEQ ID NO: 100.

SEQ ID: 100
UGCAAGUACGACAUCACCCUGCAGGAGAUCAUCAAGACCCUGAAC
CUGACCGACGGCAAGGGCAAGAACAGCUGCAUGGAGCUGACCGUG
GCCGACGCCUUCGGCCCCAAGAACACCGACGGCAAGGAGAUCUGC
CGCGCCGCCAAGGUGCUGCAGCAGUACAAGCGCCACGACCGCAGC
CUGAUCAAGGAGUGCCUGAGCGGCCUGGACCGCAACCUGAAGGGC
AUGGCCAACGGCACCUGCUGCACCGUGAACGAGGCCAAGAAGAGC
ACCCUGAAGGACUUCCUGGAGCGCCUGAAGACCAUCAUGAAGGAG
AAGUACAGCAAGUGCAGC

In one embodiment, the at least one IL-4 cytokine fragment is a peptide comprising or consisting of at least one epitope of equine IL-4.

Examples of epitopes of equine IL-4 include, but are not limited to, LQEIIKTLNLTDGKGKNSCMELT (SEQ ID NO: 109), KEICRAAKVLQQYK (SEQ ID NO: 110), RSLIKECLSGLDRNLKGMAN (SEQ ID NO: 111) and LKDFLERLKTIMKEKYSKC (SEQ ID NO: 112).

Thus, according to one embodiment, the at least one RNA molecule comprises one or more RNA sequences encoding a peptide comprising or consisting of at least one epitope of equine IL-4, such as, for example, SEQ ID NO: 109-112.

In one embodiment, one or more cysteine residue(s) are added to the extremities of peptides comprising or consisting of at least one epitope of equine IL-4. In one embodiment, one cysteine residue is added to each extremity of a peptide comprising or consisting of at least one epitope of equine IL-4, thereby conferring a cyclic conformation to said peptide.

Examples of cyclic peptides comprising at least one epitope of equine IL-4 include but are not limited to, CLQEIIKTLNLTDGKGKNSCMELTC (SEQ ID NO: 113), CKEICRAAKVLQQYKC (SEQ ID NO: 114), CRSLIKECLSGLDRNLKGMANC (SEQ ID NO: 115) and CLKDFLERLKTIMKEKYSKC (SEQ ID NO: 116).

Thus, according to one embodiment, the at least one RNA molecule comprises one or more RNA sequences encoding a cyclic peptide comprising or consisting of at least one epitope of equine IL-4, such as, for example, SEQ ID NO: 113-116.

In one embodiment, the at least one RNA sequence, in particular the at least one RNA sequence encoding equine IL-4 or at least one fragment thereof further comprises a RNA sequence encoding a signal peptide, such as, for example the signal peptide of the native equine IL-4 protein.

In one embodiment, the signal peptide is the signal peptide of equine IL-4, preferably comprising or consisting of the sequence SEQ ID NO: 84 (MGLTYQLLPALVCLLACTSFIQG), that may be encoded by the RNA sequence SEQ ID NO: 85 (AUGGGCCUGACCUACCAGCUGCUGCCCGCCCUGGUGUGCCUG CUGGCCUGCACCAGCUUCAUCCAGGGC).

Other examples of signal peptides that may be used in the present invention include, without limitation, SEQ ID NO: 88-93. Other examples of signal peptides that may be used in the present invention include, without limitation, SEQ ID NOs: 78, 103, 117, 127, 137, 140 and 142.

In one embodiment, the at least one cytokine is IL-13.

In one embodiment, IL-13 is derived from a mammalian IL-13.

In one embodiment, IL-13 is a variant of a mammal IL-13, wherein said variant present at least about 70%, 75%, 80%, 85%, 90%, 95% or more identity with the mammal IL-13 from which it derives.

In another embodiment, the at least one cytokine is a fragment of IL-13, such as, for example, a fragment of IL-13 comprising at least about 5, 6, 7, 8, 9 10, 11, 12, 13, 14 15, 16, 17, 18, 19, 20, 25, 30, 35, 40, 45, 50, 55, 60, 65, 70, 75, 80, 85, 90, 95, 100, 105, 110, 115, or 120 amino acids (preferably contiguous amino acids) of the IL-13 molecule from which it derives.

In one embodiment, the fragments may consist or comprise epitopes. In one embodiment, said fragment of IL-13 comprises or consists of at least one epitope of IL-13. Examples of epitopes of IL-13 are listed below.

In one embodiment, IL-13 is full-length IL-13.

In one embodiment of the present invention, IL-13 is human IL-13. Human IL-13 has a sequence SEQ ID NO: 23 (UniProt ID: P35225-1).

SEQ ID NO: 23
LTCLGGFASPGPVPPSTALRELIEELVNITQNQKAPLCNGSMVWS
INLTAGMYCAALESLINVSGCSAIEKTQRMLSGFCPHKVSAGQFS
SLHVRDTKIEVAQFVKDLLLHLKKLFREGREN

In one embodiment, the RNA sequence encoding the at least one cytokine comprises or consists of a sequence encoding human IL-13 protein. An example of RNA sequence encoding human IL-13 comprises or consists of SEQ ID NO: 24, or a sequence having at least about 70%, preferably at least about 75%, 80%, 85%, 90%, 95% or more identity with SEQ ID NO: 24.

SEQ ID: 24
CUGACCUGCCUGGGCGGCUUCGCCAGCCCCGGCCCCGUGCCCCCC
AGCACCGCCCUGCGCGAGCUGAUCGAGGAGCUGGUGAACAUCACC
CAGAACCAGAAGGCCCCCCUGUGCAACGGCAGCAUGGUGUGGAGC
AUCAACCUGACCGCCGGCAUGUACUGCGCCGCCCUGGAGAGCCUG
AUCAACGUGAGCGGCUGCAGCGCCAUCGAGAAGACCCAGCGCAUG
CUGAGCGGCUUCUGCCCCCACAAGGUGAGCGCCGGCCAGUUCAGC
AGCCUGCACGUGCGCGACACCAAGAUCGAGGUGGCCCAGUUCGUG
AAGGACCUGCUGCUGCACCUGAAGAAGCUGUUCCGCGAGGGCCGC
UUCAAC

In one embodiment, the at least one IL-13 cytokine fragment is a peptide comprising or consisting of at least one epitope of human IL-13.

Examples of epitopes of human IL-13 include, but are not limited to, LRELIEELVNITQNQKAPLCNG (SEQ ID NO: 25), NGSMVWSINLTAGMYCA (SEQ ID NO: 26), AGMYCAALESLINVSGCSAIEK (SEQ ID NO: 27) and VAQFVKDLLLHLKKLFREGRFN (SEQ ID NO: 28).

Thus, according to one embodiment, the at least one RNA molecule comprises one or more RNA sequences encoding a peptide comprising or consisting of at least one epitope of human IL-13, such as, for example, SEQ ID NO: 25, SEQ ID NO: 26, SEQ ID NO: 27 or SEQ ID NO: 28.

In one embodiment, one or more cysteine residue(s) are added to the extremities of peptides comprising or consisting of at least one epitope of human IL-13. In one embodiment, one cysteine residue is added to each extremity of a peptide comprising or consisting of at least one epitope of human IL-4, thereby conferring a cyclic conformation to said peptide.

Examples of cyclic peptides comprising at least one epitope of human IL-13 include, but are not limited to: CLRELIEELVNITQNQKAPLCNGC (SEQ ID NO: 29), CNGSMVWSINLTAGMYCAC (SEQ ID NO: 30), CAGMYCAALESLINVSGCSAIEKC (SEQ ID NO: 31) and CVAQFVKDLLLHLKKLFREGRFNC (SEQ ID NO: 32).

Thus, according to one embodiment, the at least one RNA molecule of the present invention comprises one or more RNA sequences encoding a cyclic peptide comprising or consisting of at least one epitope of human IL-13, such as, for example, SEQ ID NO: 29, SEQ ID NO: 30, SEQ ID NO: 31 or SEQ ID NO: 32.

In one embodiment, the at least one RNA sequence, in particular the at least one RNA sequence encoding human IL-13 or at least one fragment thereof further comprises a RNA sequence encoding a signal peptide, such as, for example the signal peptide of the native human IL-13 protein.

In one embodiment, the signal peptide is the signal peptide of human IL-13, preferably comprising or consisting of the sequence SEQ ID NO: 117 (MHPLLNPLLLALGLMALLLTTVIA), that may be encoded by the RNA sequence SEQ ID NO: 118 (AUGCACCCCCUGCUGAACCCCCUGCUGCUGGCCCUGGGC CUGAUGGCCCUGCUGCUGACCACCGUGAUCGCC).

Other examples of signal peptides that may be used in the present invention include, without limitation, SEQ ID NO: 88-93. Other examples of signal peptides that may be used in the present invention include, without limitation, SEQ ID NOs: 78, 84, 103, 127, 137, 140 and 142.

In one embodiment of the present invention, IL-13 is murine IL-13. Murine IL-13 has a sequence SEQ ID NO: 33 (UniProt ID: P20109-1).

SEQ ID NO: 33
APGPVPRSVSLPLTLKELIEELSNITQDQTPLCNGSMVWSVDLAA
GGFCVALDSLTNISNCNAIYRTQRILHGLCNRKAPTTVSSLPDTK
IEVAHFITKLLSYTKQLFRHGPF

In one embodiment, the RNA sequence encoding the at least one cytokine comprises or consists of a sequence encoding murine IL-13. An example of RNA sequence encoding murine IL-13 comprises or consists of SEQ ID NO: 34, or a sequence having at least about 70%, preferably at least about 75%, 80%, 85%, 90%, 95% or more identity with SEQ ID NO: 34.

SEQ ID: 34
GCCCCCGGCCCCGUGCCCAGGAGCGUGAGCCUGCCCCUGACCCUG
AAGGAGCUGAUCGAGGAGCUGAGCAACAUCACCCAGGACCAGACC
CCCCUGUGCAACGGCAGCAUGGUGUGGAGCGUGGACCUGGCCGCC
GGCGGCUUCUGCGUGGCCCUGGACAGCCUGACCAACAUCAGCAAC
UGCAACGCCAUCUACAGGACCCAGAGGAUCCUGCACGGCCUGUGC
AACAGGAAGGCCCCCACCACCGUGAGCAGCCUGCCCGACACCAAG
AUCGAGGUGGCCCACUUCAUCACCAAGCUGCUGAGCUACACCAAG
CAGCUGUUCAGGCACGGCCCCUUC

In one embodiment, the at least one IL-13 cytokine fragment is a peptide comprising or consisting of at least one epitope of murine IL-13.

Examples of epitopes of murine IL-13 include, but are not limited to, LKELIEELSNITQDQTPLCNG (SEQ ID NO: 35), NGSMVWSVDLAAGGFCV (SEQ ID NO: 36), AGGFCVALDSLTNISNCNAIYR (SEQ ID NO: 37) and VAHFITKLLSYTKQLFRHGPF (SEQ ID NO: 38).

Thus, according to one embodiment, the at least one RNA molecule of the present invention comprises one or more RNA sequences encoding a peptide comprising or consisting of at least one epitope of murine IL-13, such as, for example, SEQ ID NO: 35, SEQ ID NO: 36, SEQ ID NO: 37 or SEQ ID NO: 38.

In one embodiment, one or more cysteine residue(s) are added to the extremities of peptides comprising at least one epitope of murine IL-13. In one embodiment, one cysteine residue is added to each extremity of a peptide comprising or consisting of at least one epitope of murine IL-13, thereby conferring a cyclic conformation to said peptide.

Examples of cyclic peptides comprising at least one epitope of murine IL-13 include, but are not limited to, CLKELIEELSNITQDQTPLCNGC (SEQ ID NO: 39), CNGSMVWSVDLAAGGFCVC (SEQ ID NO: 40), CAGGFCVALDSLTNISNCNAIYRC (SEQ ID NO: 41) and CVAHFITKLLSYTKQLFRHGPFC (SEQ ID NO: 42).

Thus, according to one embodiment, the at least one RNA molecule of the present invention comprises one or more RNA sequences encoding a cyclic peptide comprising or consisting of at least one epitope of murine IL-13, such as, for example, SEQ ID NO: 39, SEQ ID NO: 40, SEQ ID NO: 41 or SEQ ID NO: 42.

In one embodiment, the at least one RNA sequence, in particular the at least one RNA sequence encoding murine IL-13 or at least one fragment thereof further comprises a RNA sequence encoding a signal peptide, such as, for example the signal peptide of the native murine IL-13 protein.

In one embodiment, the signal peptide is the signal peptide of murine IL-13, preferably comprising or consisting of the sequence SEQ ID NO: 142 (MALWVTAVLALACLGGLA) that may be encoded by the RNA sequence SEQ ID NO: 143 (AUGGCCCUGUGGGUGACCGCCGUGCUGGCCCUGGCCUGC CUGGGCGGCCUGGCC).

Other examples of signal peptides that may be used in the present invention include, without limitation, SEQ ID NO: 88-93. Other examples of signal peptides that may be used in the present invention include, without limitation, SEQ ID NOs: 78, 84, 103, 117, 127, 137 and 140.

In one embodiment of the present invention, IL-13 is canine IL-13. Canine IL-13 has a sequence SEQ ID NO: 43 (UniProt ID: Q9N0W9-1).

SEQ ID NO: 43
SPSPVTPSPTLKELIEELVNITQNQASLCNGSMVWSVNLTAGMYC
AALESLINVSDCSAIQRTQRMLKALCSQKPAAGQISSERSRDTKI
EVIQLVKNLLTYVRGVYRHGNFR

In one embodiment, the RNA sequence encoding the at least one cytokine comprises or consists of a sequence encoding canine IL-13. An example of RNA sequence encoding canine IL-13 comprises or consists of SEQ ID NO: 44, or a sequence having at least about 70%, preferably at least about 75%, 80%, 85%, 90%, 95% or more identity with SEQ ID NO: 44.

SEQ ID NO: 44
AGCCCCAGCCCCGUGACCCCCAGCCCCACCCUGAAGGAGCUGAUC
GAGGAGCUGGUGAACAUCACCCAGAACCAGGCCAGCCUGUGCAAC
GGCAGCAUGGUGUGGAGCGUGAACCUGACCGCCGGCAUGUACUGC
GCCGCCCUGGAGAGCCUGAUCAACGUGAGCGACUGCAGCGCCAUC
CAGCGCACCCAGCGCAUGCUGAAGGCCCUGUGCAGCCAGAAGCCC
GCCGCCGGCCAGAUCAGCAGCGAGCGCAGCCGCGACACCAAGAUC
GAGGUGAUCCAGCUGGUGAAGAACCUGCUGACCUACGUGCGCGGC
GUGUACCGCCACGGCAACUUCCGC

In one embodiment, the at least one IL-13 cytokine fragment is a peptide comprising or consisting of at least one epitope of canine IL-13.

Examples of epitopes of canine IL-13 include, but are not limited to, LKELIEELVNITQNQASLCNG (SEQ ID NO: 119), NGSMVWSVNLTAGMYCA (SEQ ID NO: 120), AGMYCAALESLINVSDCSAIQR (SEQ ID NO: 121) and VIQLVKNLLTYVRGVYRHGNF (SEQ ID NO: 122).

Thus, according to one embodiment, the at least one RNA molecule of the present invention comprises one or more RNA sequences encoding a peptide comprising or consisting of at least one epitope of canine IL-13, such as, for example, SEQ ID NO: 119-122.

In one embodiment, one or more cysteine residue(s) are added to the extremities of peptides comprising or consisting of at least one epitope of canine IL-13. In one embodiment, one cysteine residue is added to each extremity of a peptide comprising or consisting of at least one epitope of canine IL-13, thereby conferring a cyclic conformation to said peptide.

Examples of cyclic peptides comprising at least one epitope of canine IL-13 include, but are not limited to, CLKELIEELVNITQNQASLCNGC (SEQ ID NO: 123), CNGSMVWSVNLTAGMYCAC (SEQ ID NO: 124), CAGMYCAALESLINVSDCSAIQRC (SEQ ID NO: 125) and CVIQLVKNLLTYVRGVYRHGNFC (SEQ ID NO: 126).

Thus, according to one embodiment, the at least one RNA molecule of the present invention comprises one or more RNA sequences encoding a cyclic peptide comprising or consisting of at least one epitope of canine IL-13, such as, for example, SEQ ID NO: 123-126.

In one embodiment, the at least one RNA sequence, in particular the at least one RNA sequence encoding canine IL-13 or at least one fragment thereof further comprises a RNA sequence encoding a signal peptide, such as, for example the signal peptide of the native canine IL-13 protein.

In one embodiment, the signal peptide is the signal peptide of canine IL-13, preferably comprising or consisting of the sequence MALWLTVVIALTCLGGLA (SEQ ID NO: 127) that may be encoded by the RNA sequence AUGGCCCUGUGGCUGACCGUGGUGAUCGCCCUGACCUGCCUGGGCGGCCU GGCC (SEQ ID NO: 128).

Other examples of signal peptides that may be used in the present invention include, without limitation, SEQ ID NO: 88-93. Other examples of signal peptides that may be used in the present invention include, without limitation, SEQ ID NOs: 78, 84, 103, 117, 137, 140 and 142.

In one embodiment of the present invention, IL-13 is equine IL-13. Equine IL-13 has a sequence SEQ ID NO: 101 (UniProt ID: B6C802).

SEQ ID NO: 101
APLPSSMALKELIKELVNITQNQAPLCNGSMVWSVNLTADTYCRA
LESLSNVSTCSAIQNTRKMLTKLCPHQLSAGQVSSERARDTKIEV
IVLVKDLLKNLRKIFHGGKHVDA

In one embodiment, the RNA sequence encoding the at least one cytokine comprises or consists of a sequence encoding equine IL-13. An example of RNA sequence encoding equine IL-13 comprises or consists of SEQ ID NO: 102, or a sequence having at least about 70%, preferably at least about 75%, 80%, 85%, 90%, 95% or more identity with SEQ ID NO: 102.

SEQ ID: 102
GCCCCCCUGCCCAGCAGCAUGGCCCUGAAGGAGCUGAUCAAGGAG
CUGGUGAACAUCACCCAGAACCAGGCCCCCCUGUGCAACGGCAGC
AUGGUGUGGAGCGUGAACCUGACCGCCGACACCUACUGCCGCGCC
CUGGAGAGCCUGAGCAACGUGAGCACCUGCAGCGCCAUCCAGAAC
ACCCGCAAGAUGCUGACCAAGCUGUGCCCCCACCAGCUGAGCGCC
GGCCAGGUGAGCAGCGAGCGCGCCCGCGACACCAAGAUCGAGGUG
AUCGUGCUGGUGAAGGACCUGCUGAAGAACCUGCGCAAGAUCUUC
CACGGCGGCAAGCACGUGGACGCC

In one embodiment, the at least one IL-13 cytokine fragment is a peptide comprising or consisting of at least one epitope of equine IL-13.

Examples of epitopes of equine IL-13 include, but are not limited to, LKELIKELVNITQNQAPLCNG (SEQ ID NO: 129), NGSMVWSVNLTADTYCRA (SEQ ID NO: 130), ADTYCRALESLSNVSTCSAIQN (SEQ ID NO: 131) and VIVLVKDLLKNLRKIFHGGK (SEQ ID NO: 132).

Thus, according to one embodiment, the at least one RNA molecule of the present invention comprises one or more RNA sequences encoding a peptide comprising or consisting of at least one epitope of equine IL-13, such as, for example, SEQ ID NO: 129-132.

In one embodiment, one or more cysteine residue(s) are added to the extremities of peptides comprising or consisting of at least one epitope of equine IL-13. In one embodiment, one cysteine residue is added to each extremity of a peptide comprising or consisting of at least one epitope of equine IL-13, thereby conferring a cyclic conformation to said peptide.

Examples of cyclic peptides comprising at least one epitope of equine IL-13 include, but are not limited to, CLKELIKELVNITQNQAPLCNGC (SEQ ID NO: 133), CNGSMVWSVNLTADTYCRAC (SEQ ID NO: 134), CADTYCRALESLSNVSTCSAIQNC (SEQ ID NO: 135) and CVIVLVKDLLKNLRKIFHGGKC (SEQ ID NO: 136).

Thus, according to one embodiment, the at least one RNA molecule of the present invention comprises one or more RNA sequences encoding a cyclic peptide comprising or consisting of at least one epitope of equine IL-13, such as, for example, SEQ ID NO: 133-136.

In one embodiment, the at least one RNA sequence, in particular the at least one RNA sequence encoding equine IL-13 or at least one fragment thereof further comprises a RNA sequence encoding a signal peptide, such as, for example the signal peptide of the native equine IL-13 protein.

In one embodiment, the signal peptide is the signal peptide of equine IL-13, preferably comprising or consisting of the sequence SEQ ID NO: 137 (MALWLTAVIALACLGGLASP), that may be encoded by the RNA sequence SEQ ID NO: 138 (AUGGCCCUGUGGCUGACCGCCGUGAUCGCCCUGGCCUGCCU GGGCGGCCUGGCCAGCCCC).

Other examples of signal peptides that may be used in the present invention include, without limitation, SEQ ID NO: 88-93. Other examples of signal peptides that may be used in the present invention include, without limitation, SEQ ID NOs: 78, 84, 103, 117, 127, 140 and 142.

In one embodiment, the at least one RNA molecule of the invention encodes (i) at least one amino acid sequence comprising or consisting of IL-4, at least one variant of IL-4, at least one fragment of IL-4 or at least one epitope of IL-4 (in particular one of the peptides described hereinabove) and (ii) at least one amino acid sequence comprising or consisting of IL-13, at least one variant of IL-13, at least one fragment of IL-13 or at least one epitope of IL-13 (in particular one of the peptides described hereinabove).

According to the present invention, the at least one RNA molecule encodes at least one T cell epitope. In one embodiment, the at least one RNA molecule of the invention encodes an amino acid sequence comprising one T cell epitope. In one embodiment, the at least one RNA molecule of the invention encodes an amino acid sequence comprising at least 2, 3, 4, 5 or 10 T cell epitopes (e.g., 2, 3, 4, 5, 6, 7, 8, 9, 10, 11, 12, 13, 14, 15, 16, 17, 18, 19 or 20 T cell epitopes or more), that may be identical or different, and that may be linked together by at least one spacer. Examples of spacers are listed herein.

In one embodiment, the at least one T cell epitope is a dominant T cell epitope, i.e., an epitope that is not HLA-restricted. In one embodiment, the at least one T cell epitope is a Th2 epitope.

In one embodiment, the at least one T cell epitope is comprised in a peptide or protein, and the at least one RNA molecule encodes at least one peptide or protein comprising at least one T cell epitope. In one embodiment, the at least one peptide or protein comprising at least one T cell epitope is distinct from the cytokine or fragment thereof.

Examples of T cell epitopes or of peptides or proteins comprising at least one T cell epitope include, but are not limited to, the group comprising or consisting of CRM₁₉₇, variants and fragments thereof (e.g., CRM₁₉₇₍₂₉₉₋₃₁₂₎, CRM₁₉₇₍₄₂₅₋₄₄₀₎, CRM₁₉₇₍₃₀₀₋₄₅₀₎), combination of diphteria and tetanus epitopes (TpD), epitopes of tetanus toxin (TT), and universal polyepitopes CD4 (e.g., N10, N19).

In one embodiment, the at least one RNA molecule comprises a RNA sequence encoding CRM₁₉₇, or a variant or fragment thereof. CRM₁₉₇ is a non-toxic mutant of diphtheria toxin having the sequence SEQ ID NO: 48, without toxic activity due to a single base substitution (mutation from glycine to glutamic acid in position 52).

SEQ ID NO: 48
GADDVVDSSKSFVMENFSSYHGTKPGYVDSIQKGIQKPKSGTQGN
YDDDWKEFYSTDNKYDAAGYSVDNENPLSGKAGGVVKVTYPGLTK
VLALKVDNAETIKKELGLSLTEPLMEQVGTEEFIKRFGDGASRVV
LSLPFAEGSSSVEYINNWEQAKALSVELEINFETRGKRGQDAMYE
YMAQACAGNRVRRSVGSSLSCINLDWDVIRDKTKTKIESLKEHGP
IKNKMSESPNKTVSEEKAKQYLEEFHQTALEHPELSELKTVTGTN
PVFAGANYAAWAVNVAQVIDSETADNLEKTTAALSILPGIGSVMG
IADGAVHHNTEEIVAQSIALSSLMVAQAIPLVGELVDIGFAAYNF
VESIINLFQVVHNSYNRPAYSPGHKTQPFLHDGYAVSWNTVEDSI
IRTGFQGESGHDIKITAENTPLPIAGVLLPTIPGKLDVNKSKTHI
SVNGRKIRMRCRAIDGDVTFCRPKSPVYVGNGVHANLHVAFHRSS
SEKIHSNEISSDSIGVLGYQKTVDHTKVNSKLSLFFEIKS

In one embodiment, the at least one RNA molecule comprises a RNA sequence encoding a variant of CRM₁₉₇, wherein said variant presents at least about 70%, 75%, 80%, 85%, 90%, 95% or more identity with SEQ ID NO: 48. In one embodiment, said variant of CRM₁₉₇ comprises the mutation from glycine to glutamic acid in position 52 (with reference to SEQ ID NO: 48) and is thus non-toxic.

In one embodiment, the at least one RNA molecule comprises a RNA sequence encoding a full-length CRM₁₉₇.

In one embodiment, the at least one RNA molecule comprises a RNA sequence encoding a fragment of CRM₁₉₇, such as, for example, a fragment comprising at least about 1, 2, 3, 4, 5, 6, 7, 8, 9, 10, 11, 12, 13, 14, 15, 16, 17, 18, 19, 20, 21, 22, 23, 24, 25, 50, 100, 150, 200, 250, 300, 350, 400, 450, or 500 amino acids (preferably contiguous amino acids) from SEQ ID NO: 48. Specific examples of fragments of CRM₁₉₇ are listed herein.

In one embodiment, the RNA sequence encoding CRM₁₉₇ comprises or consists of SEQ ID NO: 49, or a sequence having at least about 70%, preferably at least about 75%, 80%, 85%, 90%, 95% or more identity with SEQ ID NO: 49.

SEQ ID NO: 49
GGCGCCGACGACGUGGUGGACAGCAGCAAGAGCUUCGUGAUGGAG
AACUUCAGCAGCUACCACGGCACCAAGCCCGGCUACGUGGACAGC
AUCCAGAAGGGCAUCCAGAAGCCCAAGAGCGGCACCCAGGGCAAC
UACGACGACGACUGGAAGGAGUUCUACAGCACCGACAACAAGUAC
GACGCCGCCGGCUACAGCGUGGACAACGAGAACCCCCUGAGCGGC
AAGGCCGGCGGCGUGGUGAAGGUGACCUACCCCGGCCUGACCAAG
GUGCUGGCCCUGAAGGUGGACAACGCCGAGACCAUCAAGAAGGAG
CUGGGCCUGAGCCUGACCGAGCCCCUGAUGGAGCAGGUGGGCACC
GAGGAGUUCAUCAAGAGGUUCGGCGACGGCGCCAGCAGGGUGGUG
CUGAGCCUGCCCUUCGCCGAGGGCAGCAGCAGCGUGGAGUACAUC
AACAACUGGGAGCAGGCCAAGGCCCUGAGCGUGGAGCUGGAGAUC
AACUUCGAGACCAGGGGCAAGAGGGGCCAGGACGCCAUGUACGAG
UACAUGGCCCAGGCCUGCGCCGGCAACAGGGUGAGGAGGAGCGUG
GGCAGCAGCCUGAGCUGCAUCAACCUGGACUGGGACGUGAUCAGG
GACAAGACCAAGACCAAGAUCGAGAGCCUGAAGGAGCACGGCCCC
AUCAAGAACAAGAUGAGCGAGAGCCCCAACAAGACCGUGAGCGAG
GAGAAGGCCAAGCAGUACCUGGAGGAGUUCCACCAGACCGCCCUG
GAGCACCCCGAGCUGAGCGAGCUGAAGACCGUGACCGGCACCAAC
CCCGUGUUCGCCGGCGCCAACUACGCCGCCUGGGCCGUGAACGUG
GCCCAGGUGAUCGACAGCGAGACCGCCGACAACCUGGAGAAGACC
ACCGCCGCCCUGAGCAUCCUGCCCGGCAUCGGCAGCGUGAUGGGC
AUCGCCGACGGCGCCGUGCACCACAACACCGAGGAGAUCGUGGCC
CAGAGCAUCGCCCUGAGCAGCCUGAUGGUGGCCCAGGCCAUCCCC
CUGGUGGGCGAGCUGGUGGACAUCGGCUUCGCCGCCUACAACUUC
GUGGAGAGCAUCAUCAACCUGUUCCAGGUGGUGCACAACAGCUAC
AACAGGCCCGCCUACAGCCCCGGCCACAAGACCCAGCCCUUCCUG
CACGACGGCUACGCCGUGAGCUGGAACACCGUGGAGGACAGCAUC
AUCAGGACCGGCUUCCAGGGCGAGAGCGGCCACGACAUCAAGAUC
ACCGCCGAGAACACCCCCCUGCCCAUCGCCGGCGUGCUGCUGCCC
ACCAUCCCCGGCAAGCUGGACGUGAACAAGAGCAAGACCCACAUC
AGCGUGAACGGCAGGAAGAUCAGGAUGAGGUGCAGGGCCAUCGAC
GGCGACGUGACCUUCUGCAGGCCCAAGAGCCCCGUGUACGUGGGC
AACGGCGUGCACGCCAACCUGCACGUGGCCUUCCACAGGAGCAGC
AGCGAAAAGAUCCACAGCAACGAGAUCAGCAGCGACAGCAUCGGC
GUGCUGGGCUACCAGAAGACCGUGGACCACACCAAGGUGAACAGC
AAGCUGAGCCUGUUCUUCGAGAUCAAGAGC

In one embodiment, the at least one RNA molecule comprises a RNA sequence encoding a fragment of CRM₁₉₇ comprising or consisting of amino acid 299 to amino acid 312 of SEQ ID NO: 48. The fragment of CRM₁₉₇ consisting of amino acid 299 to amino acid 312 of SEQ ID NO: 48 is referred to as CRM₁₉₇₍₂₉₉₋₃₁₂₎ and consists of the sequence SEQ ID NO: 50 (KTTAALSILPGIGS).

In one embodiment, the RNA molecule of the present invention comprises a RNA sequence encoding a variant of SEQ ID NO: 50, wherein said variant presents at least about 70%, 75%, 80%, 85%, 90%, 95% or more identity with SEQ ID NO: 50.

In one embodiment, the at least one RNA molecule comprises a RNA sequence encoding a fragment of CRM₁₉₇₍₂₉₉₋₃₁₂₎ comprising or consisting of at least about 5, 6, 7, 8, 9, 10, 11, 12 or 13 amino acids (preferably contiguous amino acids) of SEQ ID NO: 50.

In one embodiment, the RNA sequence encoding the at least one T cell epitope or peptide or protein comprising at least one T cell epitope comprises or consists of a sequence encoding CRM₁₉₇₍₂₉₉₋₃₁₂₎. An example of RNA sequence encoding CRM₁₉₇₍₂₉₉₋₃₁₂₎ comprises or consists of SEQ ID NO: 51, or a sequence having at least about 70%, preferably at least about 75%, 80%, 85%, 90%, 95% or more identity with SEQ ID NO: 51 (AAGACCACCGCCGCCCUGAGCAUCCUGCCCGGCAUCGGCAGC).

In one embodiment, the at least one RNA molecule comprises a RNA sequence encoding a fragment of CRM₁₉₇ comprising or consisting of amino acid 425 to amino acid 440 of SEQ ID NO: 48. The fragment of CRM₁₉₇ consisting of amino acid 425 to amino acid 440 of SEQ ID NO: 48 is referred to as CRM₁₉₇₍₄₂₅₋₄₄₀₎ and consists of the sequence SEQ ID NO: 52 (TPLPIAGVLLPTIPGK).

In one embodiment, the RNA molecule of the present invention comprises a RNA sequence encoding a variant of SEQ ID NO: 52, wherein said variant presents at least about 70%, 75%, 80%, 85%, 90%, 95% or more identity with SEQ ID NO: 52.

In one embodiment, the at least one RNA molecule comprises a RNA sequence encoding a fragment of CRM₁₉₇₍₄₂₅₋₄₄₀₎ comprising or consisting of at least about 5, 6, 7, 8, 9, 10, 11, 12, 13, 14 or 15 amino acids (preferably contiguous amino acids) of SEQ ID NO: 52.

In one embodiment, the RNA sequence encoding the at least one T cell epitope or peptide or protein comprising at least one T cell epitope comprises or consists of a sequence encoding CRM₁₉₇₍₄₂₅₋₄₄₀₎. An example of RNA sequence encoding CRM₁₉₇₍₄₂₅₋₄₄₀₎ comprises or consists of SEQ ID NO: 53, or a sequence having at least about 70%, preferably at least about 75%, 80%, 85%, 90%, 95% or more identity with SEQ ID NO: 53 (ACCCCCCUGCCCAUCGCCGGCGUGCUGCUGCCCACCAUCCCCGGCAAG).

In one embodiment, the at least one RNA molecule comprises a RNA sequence encoding a fragment of CRM₁₉₇ comprising or consisting of amino acid 300 to amino acid 450 of SEQ ID NO: 48. The fragment of CRM₁₉₇ consisting of amino acid 300 to amino acid 450 of SEQ ID NO: 48 is referred to as CRM₁₉₇₍₃₀₀₋₄₅₀₎ and consists of the sequence SEQ ID NO: 46.

SEQ ID NO: 46
TAALSILPGIGSVMGIADGAVHHNTEEIVAQSIALSSLMVAQAIP
LVGELVDIGFAAYNFVESIINLFQVVHNSYNRPAYSPGHKTQPFL
HDGYAVSWNTVEDSIIRTGFQGESGHDIKITAENTPLPIAGVLLP
TIPGKLDVNKSKTHI

In one embodiment, the RNA molecule of the present invention comprises a RNA sequence encoding a variant of SEQ ID NO: 46, wherein said variant presents at least about 70%, 75%, 80%, 85%, 90%, 95% or more identity with SEQ ID NO: 46.

In one embodiment, the at least one RNA molecule comprises a RNA sequence encoding a fragment of CRM₁₉₇₍₃₀₀₋₄₅₀₎ comprising or consisting of at least about 5, 10, 15, 20, 25, 30, 35, 40, 45, 50, 55, 60, 65, 70, 75, 80, 85, 90, 95, 100, 105, 110, 115, 120, 125, 130, 135, 140 or 145 amino acids (preferably contiguous amino acids) of SEQ ID NO: 46.

In one embodiment, the RNA sequence encoding the at least one T cell epitope or peptide or protein comprising at least one T cell epitope comprises or consists of a sequence encoding CRM₁₉₇₍₃₀₀₋₄₅₀₎. An example of RNA sequence encoding CRM₁₉₇₍₃₀₀₋₄₅₀₎ comprises or consists of SEQ ID NO: 47, or a sequence having at least about 70%, preferably at least about 75%, 80%, 85%, 90%, 95% or more identity with SEQ ID NO: 47.

SEQ ID NO: 47
ACCGCCGCCCUGAGCAUCCUGCCCGGCAUCGGCAGCGUGAUGGGC
AUCGCCGACGGCGCCGUGCACCACAACACCGAGGAGAUCGUGGCC
CAGAGCAUCGCCCUGAGCAGCCUGAUGGUGGCCCAGGCCAUCCCC
CUGGUGGGCGAGCUGGUGGACAUCGGCUUCGCCGCCUACAACUUC
GUGGAGAGCAUCAUCAACCUGUUCCAGGUGGUGCACAACAGCUAC
AACAGGCCCGCCUACAGCCCCGGCCACAAGACCCAGCCCUUCCUG
CACGACGGCUACGCCGUGAGCUGGAACACCGUGGAGGACAGCAUC
AUCAGGACCGGCUUCCAGGGCGAGAGCGGCCACGACAUCAAGAUC
ACCGCCGAGAACACCCCCCUGCCCAUCGCCGGCGUGCUGCUGCCC
ACCAUCCCCGGCAAGCUGGACGUGAACAAGAGCAAGACCCACAUC

In one embodiment, the at least one RNA molecule comprises a RNA sequence encoding TpD. TpD comprises both diphteria and tetanus epitopes and optionally a cathepsin cleavage site.

In one embodiment, TpD comprises or consists of the sequence SEQ ID NO: 54 (ILMQYIKANSKFIGIPMGLPQSIALSSLMVAQ).

In one embodiment, the at least one RNA molecule comprises a RNA sequence encoding a variant of SEQ ID NO: 54, wherein said variant presents at least about 70%, 75%, 80%, 85%, 90%, 95% or more identity with SEQ ID NO: 54.

In one embodiment, the at least one RNA molecule comprises a RNA sequence encoding full-length TpD. An example of RNA sequence encoding full-length TpD is a RNA sequence comprising or consisting of SEQ ID NO: 55, or a sequence having at least about 70%, preferably at least about 75%, 80%, 85%, 90%, 95% or more identity with SEQ ID NO: 55.

SEQ ID NO 55
AUCCUGAUGCAGUACAUCAAGGCCAACAGCAAGUUCAUCGGCAUC
CCCAUGGGCCUGCCCCAGAGCAUCGCCCUGAGCAGCCUGAUGGUG
GCCCAG

In one embodiment, the at least one RNA molecule comprises a RNA sequence encoding a fragment of TpD, such as, for example, a fragment comprising at least about 5, 10, 15, 20, 25 or 30 amino acids (preferably contiguous amino acids) from SEQ ID NO: 54.

In one embodiment, the at least one RNA molecule of the invention comprises a RNA sequence encoding a “p2-spacer-p30” construct. P2-spacer-p30 is a peptide comprising two epitopes of tetanus toxin (TT), i.e., peptides P2 and peptide P30, and at least one spacer.

In one embodiment, the spacer comprises from 1 to 20 amino acids. In one embodiment, the spacer consists of two amino acids. In one embodiment, the spacer consists of RR.

In one embodiment, p2-spacer-p30 comprises or consists of a peptide “p2TT” consisting of the sequence SEQ ID NO: 63, a peptide “p30TT” consisting of the sequence SEQ ID NO: 62, and optionally a spacer (such as, for example, RR).

In one embodiment, p2-spacer-p30 comprises or consists of a construct p2-RR-p30, i.e., a construct comprising or consisting of peptides p2TT and p30TT and of the spacer RR, and having or comprising the sequence SEQ ID NO: 56 or a variant thereof, having preferably at least about 75%, 80%, 85%, 90%, 95% or more identity with SEQ ID NO: 56. A p2-RR-p30 constructs.

SEQ ID NO: 56
QYIKANSKFIGITERRFNNFTVSFWLRVPKVSASHLEQY

In one embodiment, the at least one T cell epitope or peptide or protein comprising at least one T cell epitope or a fragment thereof is full-length p2-RR-p30.

In one embodiment, the at least one T cell epitope or peptide or protein comprising at least one T cell epitope or a fragment thereof is a fragment of p2-RR-p30, comprising at least about 5, 10, 15, 20, 25, 30, 32, 35 or 37 amino acids (preferably contiguous amino acids) from SEQ ID NO: 56.

In one embodiment, the RNA molecule of the invention comprises a RNA sequence encoding p2-RR-p30. An example of RNA sequence encoding p2-RR-p30 is a sequence comprising or consisting SEQ ID NO: 57, or a sequence having at least about 70%, preferably at least about 75%, 80%, 85%, 90%, 95% or more identity with SEQ ID NO: 57.

SEQ ID NO: 57
CAGUACAUCAAGGCCAACAGCAAGUUCAUCGGCAUCACCGAGAGG
AGGUUCAACAACUUCACCGUGAGCUUCUGGCUGAGGGUGCCCAAG
GUGAGCGCCAGCCACCUGGAGCAGUAC

In one embodiment, the at least one RNA molecule of the invention comprises a RNA sequence encoding one or more universal CD4 polyepitopes. In one embodiment, the at least one RNA molecule of the invention comprises a RNA sequence encoding at least two universal CD4 polyepitopes, optionally linked by spacer(s). Examples of universal CD4 polyepitopes include, but are not limited to, peptides having the sequences SEQ ID NO: 58 to 67 (Table 1). Examples of spacers are provided herein.

TABLE 1
Universal CD4 polyepitopes amino acid sequences

| CD4 T cell | |
|---|---|
| epitope | Sequence (SEQ ID numbers) |
| p23TT | VSIDKFRIFCKANPK (SEQ ID NO: 58) |
| p32TT | LKFIIKRYTPNNEIDS (SEQ ID NO: 59) |
| p21TT | IREDNNITLKLDRCNN (SEQ ID NO: 60) |
| PfCs | EKKIAKMEKASSVFNVVN (SEQ ID NO: 61) |
| p30TT | FNNFTVSFWLRVPKVSASHLE |
| | (SEQ ID NO: 62) |
| p2TT | QYIKANSKFIGITE (SEQ ID NO: 63) |
| HBVnc | PHHTALRQAILCWGELMTLA |
| | (SEQ ID NO: 64) |
| HA | PKYVKONTLKLAT (SEQ ID NO: 65) |
| HbsAg | FFLLTRILTIPQSLD (SEQ ID NO: 66) |
| MT | YSGPLKAEIAQRLEDV (SEQ ID NO: 67) |

TT: Tetanus toxin, PfCs: P. falciparum circumsporozoite protein, HBVnc: HBV nucleocapsid, HbsAg: HBV Surface antigen, HA: Influenza virus hemagglutinin, MT: Influenza virus Matrix protein

In one embodiment, the at least one T cell epitope is a variant of a sequence selected from SEQ ID NO: 58-67, wherein said variant presents at least about 70%, 75%, 80%, 85%, 90%, 95% or more identity with SEQ ID NO: 58-67.

In one embodiment, the at least one T cell epitope is a full-length universal CD4 protein polyepitope as disclosed in Table 1.

In one embodiment, the at least one T cell epitope is a fragment of a universal CD4 polyepitope comprising at least about 5, 6, 7, 8, 9, 10, 11 or 12 amino acids (preferably contiguous amino acids) of SEQ ID NO: 58-60, 63, and 65-67 or at least about 5, 6, 7, 8, 9, 10, 11, 12, 13, 14, 15, 16 or 17 amino acids (preferably contiguous amino acids) of SEQ ID NO: 61-62 and 64.

In one embodiment, the RNA sequence encoding the at least one T cell epitope comprises or consists of a sequence encoding at least two universal CD4 polyepitopes, optionally linked by spacer(s). Examples of RNA sequences encoding universal CD4 polyepitopes include, but are not limited to, RNA sequence comprising or consisting in the sequences SEQ ID NO: 68 to 77 (Table 2).

TABLE 2
Universal CD4 polyepitopes nucleotide sequences

| CD4 T cell | |
|---|---|
| epitope | Sequence (SEQ ID numbers) |
| p23TT | GUG AGC AUC GAC AAG UUC AGG AUC UUC UGC AAG |
| | GCC AAC CCC AAG (SEQ ID NO: 68) |
| p32TT | CUG AAG UUC AUC AUC AAG AGG UAC ACC CCC AAC |
| | AAC GAG AUC GAC AGC (SEQ ID NO: 69) |
| p21TT | AUC AGG GAG GAC AAC AAC AUC ACC CUG AAG CUG |
| | GAC AGG UGC AAC AAC (SEQ ID NO: 70) |
| PfCs | GAA AAG AAG AUC GCC AAG AUG GAG AAG GCC AGC |
| | AGC GUG UUC AAC GUG GUG AAC (SEQ ID NO: 71) |
| p30TT | UUC AAC AAC UUC ACC GUG AGC UUC UGG CUG AGG |
| | GUG CCC AAG GUG AGC GCC AGC CAC CUG GAG |
| | (SEQ ID NO: 72) |
| p2TT | CAG UAC AUC AAG GCC AAC AGC AAG UUC AUC GGC |
| | AUC ACC GAA (SEQ ID NO: 73) |
| HBVnc | CCC CAC CAC ACC GCC CUG AGG CAG GCC AUC CUG |
| | UGC UGG GGC GAG CUG AUG ACC CUG GCC |
| | (SEQ ID NO: 74) |
| HA | CCC AAG UAC GUG AAG CAG AAC ACC CUG AAG CUG |
| | GCC ACC (SEQ ID NO: 75) |
| HbsAg | UUC UUC CUG CUG ACC AGG AUC CUG ACC AUC CCC |
| | CAG AGC CUG GAC (SEQ ID NO: 76) |
| MT | UAC AGC GGC CCC CUG AAG GCC GAG AUC GCC CAG |
| | AGG CUG GAG G?C GUG (SEQ ID NO: 77) |

TT : Tetanus toxin, PfCs: P. falciparum circumsporozoite protein, HBVnc: HBV nucleocapsid, HbsAg: HBV Surface antigen, HA: Influenza virus hemagglutinin, MT: Influenza virus Matrix protein

In one embodiment, the RNA molecule of the invention comprises a RNA sequence encoding an amino acid sequence comprising at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 universal CD4 polyepitopes from Table 1 optionally linked by spacers.

In one embodiment, the at least one RNA molecule of the invention comprises a RNA sequence encoding at least two identical or different universal CD4 polyepitopes.

In one embodiment, the RNA molecule of the invention comprises a RNA sequence encoding a recombinant peptide or protein comprising or consisting of at least one T cell epitope, such as, for example, N10, N19 comprising or consisting of 10 or 19 human CD4+ T cell epitopes (respectively) derived from pathogen-derived antigens, including without limitation, TT and proteins from Plasmodium falciparum, influenza virus and hepatitis B virus (as listed in Table 1).

In one embodiment, the at least one RNA molecule of the invention comprises a RNA sequence encoding 10 universal CD4 epitopes as described herein (and is herein referred to as N10), optionally wherein the universal CD4 polyepitopes are linked by a spacer. Examples of spacers are listed herein.

A non-limitative example of N10 construct is a peptide of sequence SEQ ID NO: 80.

SEQ ID NO: 80
VSIDKFRIFCKANPKKGLKFIIKRYTPNNEIDSKGIREDNNITLK
LDRCNNKGEKKIAKMEKASSVFNVVNKGFNNFTVSFWLRVPKVSA
SHLEKGQYIKANSKFIGITEKGPHHTALRQAILCWGELMTLAKGP
KYVKQNTLKLATKGFFLLTRILTIPQSLDKGYSGPLKAEIAQRLE
DV

In one embodiment, the RNA molecule comprises a RNA sequence encoding a variant of SEQ ID NO: 80, wherein said variant presents at least about 70%, 75%, 80%, 85%, 90%, 95% or more identity with SEQ ID NO: 80.

In one embodiment, the RNA molecule comprises a RNA sequence encoding full-length N10 construct. In another embodiment, the RNA molecule comprises a RNA sequence encoding a fragment of N10, such as, for example, a fragment comprising at least about 10, 15, 20, 25, 50, 75, 100, 125, 150 or 175 amino acids (preferably contiguous amino acids) from SEQ ID NO: 80.

In one embodiment, the RNA sequence encoding at least one T cell epitope comprises or consists of a sequence encoding N10. An example of RNA sequence encoding N10 is a sequence comprising or consisting of SEQ ID NO: 81, or a sequence having at least about 70%, preferably at least about 75%, 80%, 85%, 90%, 95% or more identity with SEQ ID NO: 81.

SEQ ID NO: 81
GUGAGCAUCGACAAGUUCAGGAUCUUCUGCAAGGCCAACCCCAAG
AAGGGCCUGAAGUUCAUCAUCAAGAGGUACACCCCCAACAACGAG
AUCGACAGCAAGGGCAUCAGGGAGGACAACAACAUCACCCUGAAG
CUGGACAGGUGCAACAACAAGGGCGAAAAGAAGAUCGCCAAGAUG
GAGAAGGCCAGCAGCGUGUUCAACGUGGUGAACAAGGGCUUCAAC
AACUUCACCGUGAGCUUCUGGCUGAGGGUGCCCAAGGUGAGCGCC
AGCCACCUGGAGAAGGGCCAGUACAUCAAGGCCAACAGCAAGUUC
AUCGGCAUCACCGAAAAGGGCCCCCACCACACCGCCCUGAGGCAG
GCCAUCCUGUGCUGGGGCGAGCUGAUGACCCUGGCCAAGGGCCCC
AAGUACGUGAAGCAGAACACCCUGAAGCUGGCCACCAAGGGCUUC
UUCCUGCUGACCAGGAUCCUGACCAUCCCCCAGAGCCUGGACAAG
GGCUACAGCGGCCCCCUGAAGGCCGAGAUCGCCCAGAGGCUGGAG
GACGUG

In one embodiment, the at least one RNA molecule of the invention comprises a RNA sequence encoding 19 universal CD4 epitopes as described herein (and is herein referred to as N19), optionally wherein the universal CD4 polyepitopes are linked by a spacer. Examples of spacers are listed herein.

A non-limitative example of N19 construct is a peptide of sequence SEQ ID NO: 82.

SEQ ID NO: 82
VSIDKFRIFCKANPKKGLKFIIKRYTPNNEIDSKGIREDNNITLK
LDRCNNKGEKKIAKMEKASSVFNVVNKGFNNFTVSFWLRVPKVSA
SHLEKGQYIKANSKFIGITEKGPHHTALRQAILCWGELMTLAKGP
KYVKONTLKLATKGFFLLTRILTIPQSLDKGYSGPLKAEIAQRLE
DVKGVSIDKFRIFCKANPKKGLKFIIKRYTPNNEIDSKGIREDNN
ITLKLDRCNNKGEKKIAKMEKASSVFNVVNKGFNNFTVSFWLRVP
KVSASHLEKGQYIKANSKFIGITEKGPHHTALRQAILCWGELMTL
AKGPKYVKQNTLKLATKGFFLLTRILTIPQSLDKG

In one embodiment, the RNA molecule comprises a RNA sequence encoding a variant of SEQ ID NO: 82, wherein said variant presents at least about 70%, 75%, 80%, 85%, 90%, 95% or more identity with SEQ ID NO: 82.

In one embodiment, the RNA molecule comprises a RNA sequence encoding full-length N19 construct. In another embodiment, the RNA molecule comprises a RNA sequence encoding a fragment of N19, such as, for example, a fragment comprising at least about 10, 15, 20, 25, 50, 100, 150, 200, 250, 300 or 330 amino acids (preferably contiguous amino acids) from SEQ ID NO: 82.

In one embodiment, the RNA sequence encoding at least one T cell epitope comprises or consists of a sequence encoding N19. An example of RNA sequence encoding N19 is a sequence comprising or consisting of SEQ ID NO: 83, or a sequence having at least about 70%, preferably at least about 75%, 80%, 85%, 90%, 95% or more identity with SEQ ID NO: 83.

SEQ ID NO: 83
GUGAGCAUCGACAAGUUCAGGAUCUUCUGCAAGGCCAACCCCAAG
AAGGGCCUGAAGUUCAUCAUCAAGAGGUACACCCCCAACAACGAG
AUCGACAGCAAGGGCAUCAGGGAGGACAACAACAUCACCCUGAAG
CUGGACAGGUGCAACAACAAGGGCGAAAAGAAGAUCGCCAAGAUG
GAGAAGGCCAGCAGCGUGUUCAACGUGGUGAACAAGGGCUUCAAC
AACUUCACCGUGAGCUUCUGGCUGAGGGUGCCCAAGGUGAGCGCC
AGCCACCUGGAGAAGGGCCAGUACAUCAAGGCCAACAGCAAGUUC
AUCGGCAUCACCGAAAAGGGCCCCCACCACACCGCCCUGAGGCAG
GCCAUCCUGUGCUGGGGCGAGCUGAUGACCCUGGCCAAGGGCCCC
AAGUACGUGAAGCAGAACACCCUGAAGCUGGCCACCAAGGGCUUC
UUCCUGCUGACCAGGAUCCUGACCAUCCCCCAGAGCCUGGACAAG
GGCUACAGCGGCCCCCUGAAGGCCGAGAUCGCCCAGAGGCUGGAG
GACGUGAAGGGCGUGAGCAUCGACAAGUUCAGGAUCUUCUGCAAG
GCCAACCCCAAGAAGGGCCUGAAGUUCAUCAUCAAGAGGUACACC
CCCAACAACGAGAUCGACAGCAAGGGCAUCAGGGAGGACAACAAC
AUCACCCUGAAGCUGGACAGGUGCAACAACAAGGGCGAAAAGAAG
AUCGCCAAGAUGGAGAAGGCCAGCAGCGUGUUCAACGUGGUGAAC
AAGGGCUUCAACAACUUCACCGUGAGCUUCUGGCUGAGGGUGCCC
AAGGUGAGCGCCAGCCACCUGGAGAAGGGCCAGUACAUCAAGGCC
AACAGCAAGUUCAUCGGCAUCACCGAAAAGGGCCCCCACCACACC
GCCCUGAGGCAGGCCAUCCUGUGCUGGGGCGAGCUGAUGACCCUG
GCCAAGGGCCCCAAGUACGUGAAGCAGAACACCCUGAAGCUGGCC
ACCAAGGGCUUCUUCCUGCUGACCAGGAUCCUGACCAUCCCCCAG
AGCCUGGACAAGGGC

In one embodiment, the at least one RNA molecule of the invention comprises at least one RNA sequence encoding a spacer. In one embodiment, the cytokine is linked to the at least one T cell epitope by a spacer. In one embodiment, the cytokine is linked to the at least one T cell epitope by one or more spacers (e.g., 1, 2, 3, 4 or 5). Spacers may also be present to link T cell epitopes, or cytokines fragments.

In one embodiment, the spacer is cleavable. In one embodiment, the spacer is cleavable by a protease, such as, for example, cathepsin.

In one embodiment, the spacer is non-cleavable.

Examples of spacers include, but are not limited to PMGLP (SEQ ID NO: 86), cathepsin cleavage sites, amino acids doublets, GP, LG, GS, GPGPG (SEQ ID NO: 45), GGSGGGGSGG (SEQ ID NO: 98), (GGGGS)ₙ wherein n ranges from 1 to 4 ((SEQ ID NO: 139)ₙ), ASG, KG and RR.

In one embodiment, the spacer comprises or consists of the amino acid sequence of PMGLP (SEQ ID NO: 86), that may be encoded by the nucleic acid sequence SEQ ID NO: 87 (CCCAUGGGCCUGCCC) or a nucleic acid sequence with at least about 70%, 75%, 80%, 85%, 90% or 99% identity to SEQ ID NO: 87.

In one embodiment, a short oligo- or polypeptide spacer, having a length ranging from, e.g., 2 and 10 amino acids, may form the spacer. In one embodiment, the linker consists of 2, 3, 4, or 5 amino acids.

Examples of short spacers include but are not limited to GP, LG, ASG, KG and RR.

In one embodiment, the short spacers GP, LG, ASG, RR and KG may respectively be encoded by the RNA sequence listed in Table 3.

TABLE 3
Spacer amino acid sequences

| CD4 T cell epitope | Sequence |
|---|---|
| GP | GGCCCC |
| LG | CUGGGC |
| ASG | GCCAGCGGC |
| KG | AAGGGC |
| RR | AGGAGG |

In one embodiment, the at least one RNA molecule of the invention encodes an amino acid sequence comprising or consisting of human IL-4 (preferably having the sequence of SEQ ID NO: 1) and CRM₁₉₇ (SEQ ID NO: 48) and optionally a spacer. In one embodiment, the at least one RNA molecule encodes a GP spacer. In one embodiment, the at least one RNA molecule further encodes a signal peptide (preferably having the sequence of SEQ ID NO: 103). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 103, human IL-4 having preferably the sequence of SEQ ID NO: 1, a GP spacer and CRM₁₉₇ having preferably the sequence of SEQ ID NO: 48.

In one embodiment, the at least one RNA molecule comprises or consists of a RNA sequence encoding human IL-4 (preferably a RNA sequence of SEQ ID NO: 2) and CRM₁₉₇ (preferably a RNA sequence of SEQ ID NO: 49) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 104). In one embodiment, the RNA molecule of the invention comprises or consists of, from 5′ to 3′: a RNA sequence encoding a signal peptide having preferably the sequence of SEQ ID NO: 104, a RNA sequence encoding human IL-4 having preferably the sequence of SEQ ID NO: 2, a RNA sequence encoding a GP spacer having preferably the sequence GGCCCC and a RNA sequence encoding CRM₁₉₇ having preferably the sequence of SEQ ID NO: 49.

In one embodiment, the at least one RNA molecule of the invention encodes an amino acid sequence comprising or consisting of human IL-4 (preferably having the sequence of SEQ ID NO: 1) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of human IL-4 (preferably having the sequence of SEQ ID NO: 1) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of human IL-4 (preferably having the sequence of SEQ ID NO: 1) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of human IL-4 (preferably having the sequence of SEQ ID NO: 1) and TpD (preferably having the sequence of SEQ ID NO: 54) and optionally a spacer. In one embodiment, the at least one RNA molecule encodes a GP spacer. In one embodiment, the at least one RNA molecule further encodes a signal peptide (preferably having the sequence of SEQ ID NO: 103). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 103, human IL-4 having preferably the sequence of SEQ ID NO: 1, a GP spacer and TpD having preferably the sequence of SEQ ID NO: 54.

In one embodiment, the at least one RNA molecule comprises or consists of a RNA sequence encoding human IL-4 (preferably a RNA sequence of SEQ ID NO: 2) and TpD (preferably a RNA sequence of SEQ ID NO: 55) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 104). In one embodiment, the RNA molecule of the invention comprises or consists of, from 5′ to 3′: a RNA sequence encoding a signal peptide having preferably the sequence of SEQ ID NO: 104, a RNA sequence encoding human IL-4 having preferably the sequence of SEQ ID NO: 2, a RNA sequence encoding a GP spacer having preferably the sequence GGCCCC and a RNA sequence encoding TpD having preferably the sequence of SEQ ID NO: 55.

In one embodiment, the at least one RNA molecule of the invention encodes an amino acid sequence comprising or consisting of human IL-4 (preferably having the sequence of SEQ ID NO: 1) and p2-RR-p30 (TT) (SEQ ID NO: 56) and optionally a spacer.

In one embodiment, the at least one RNA molecule of the invention encodes an amino acid sequence comprising or consisting of human IL-4 (preferably having the sequence of SEQ ID NO: 1) and a N10 construct (preferably having the sequence of SEQ ID NO: 80) and optionally a spacer.

In one embodiment, the at least one RNA molecule of the invention encodes an amino acid sequence comprising or consisting of human IL-4 (preferably having the sequence of SEQ ID NO: 1) and a N19 construct (preferably having the sequence of SEQ ID NO: 82) and optionally a spacer. In one embodiment, the at least one RNA molecule encodes a GP spacer. In one embodiment, the at least one RNA molecule further encodes a signal peptide (preferably having the sequence of SEQ ID NO: 103). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 103, human IL-4 having preferably the sequence of SEQ ID NO: 1, a GP spacer and a N19 construct having preferably the sequence of SEQ ID NO: 82.

In one embodiment, the at least one RNA molecule comprises or consists of a RNA sequence encoding human IL-4 (preferably a RNA sequence of SEQ ID NO: 2) and a N19 construct (preferably having the sequence of SEQ ID NO: 83) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 104). In one embodiment, the RNA molecule of the invention comprises or consists of, from 5′ to 3′: a RNA sequence encoding a signal peptide having preferably the sequence of SEQ ID NO: 104, a RNA sequence encoding human IL-4 having preferably the sequence of SEQ ID NO: 2, a RNA sequence encoding a GP spacer having preferably the sequence GGCCCC and a RNA sequence encoding a N19 construct having preferably the sequence of SEQ ID NO: 83.

In one embodiment, the at least one RNA molecule of the invention encodes an amino acid sequence comprising or consisting of human IL-4 (preferably having the sequence of SEQ ID NO: 1) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and CRM₁₉₇ (SEQ ID NO: 48) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of human IL-4 (preferably a peptide having or comprising a sequence selected from SEQ ID NO: 3, 4, 5 or 6) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of murine IL-4 (preferably having the sequence of SEQ ID NO: 11) and CRM₁₉₇ (SEQ ID NO: 48) and optionally a spacer. In one embodiment, the at least one RNA molecule encodes a GP spacer. In one embodiment, the at least one RNA molecule further encodes a signal peptide (preferably having the sequence of SEQ ID NO: 140). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 140, murine IL-4 having preferably the sequence of SEQ ID NO: 11, a GP spacer and CRM₁₉₇ having preferably the sequence of SEQ ID NO: 48.

In one embodiment, the at least one RNA molecule comprises or consists of a RNA sequence encoding murine IL-4 (preferably a RNA sequence of SEQ ID NO: 12) and CRM₁₉₇ (preferably a RNA sequence of SEQ ID NO: 49) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 141). In one embodiment, the RNA molecule of the invention comprises or consists of, from 5′ to 3′: a RNA sequence encoding a signal peptide having preferably the sequence of SEQ ID NO: 141, a RNA sequence encoding murine IL-4 having preferably the sequence of SEQ ID NO: 12, a RNA sequence encoding a GP spacer having preferably the sequence GGCCCC and a RNA sequence encoding CRM₁₉₇ having preferably the sequence of SEQ ID NO: 49.

In one embodiment, the at least one RNA molecule of the invention comprises or consists in the sequence of SEQ ID NO: 146.

In one embodiment, the at least one RNA molecule is a variant of SEQ ID NO: 146, wherein said variant present at least about 70%, 75%, 80%, 85%, 90%, 95% or more identity with the SEQ ID NO: 146 from which it derives.

SEQ ID NO: 146
AUGGGCCUGAACCCCCAGCUGGUGGUGAUCCUGCUGUUCUUCCUG
GAGUGCACCAGGAGCCACAUCCACGGCUGCGACAAGAACCACCUG
AGGGAGAUCAUCGGCAUCCUGAACGAGGUGACCGGCGAGGGCACC
CCCUGCACCGAGAUGGACGUGCCCAACGUGCUGACCGCCACCAAG
AACACCACCGAGAGCGAGCUGGUGUGCAGGGCCAGCAAGGUGCUG
AGGAUCUUCUACCUGAAGCACGGCAAGACCCCCUGCCUGAAGAAG
AACAGCAGCGUGCUGAUGGAGCUGCAGAGGCUGUUCAGGGCCUUC
AGGUGCCUGGACAGCAGCAUCAGCUGCACCAUGAACGAGAGCAAG
AGCACCAGCCUGAAGGACUUCCUGGAGAGCCUGAAGAGCAUCAUG
CAGAUGGACUACAGCGGCCCCGGCGCCGACGACGUGGUGGACAGC
AGCAAGAGCUUCGUGAUGGAGAACUUCAGCAGCUACCACGGCACC
AAGCCCGGCUACGUGGACAGCAUCCAGAAGGGCAUCCAGAAGCCC
AAGAGCGGCACCCAGGGCAACUACGACGACGACUGGAAGGAGUUC
UACAGCACCGACAACAAGUACGACGCCGCCGGCUACAGCGUGGAC
AACGAGAACCCCCUGAGCGGCAAGGCCGGCGGCGUGGUGAAGGUG
ACCUACCCCGGCCUGACCAAGGUGCUGGCCCUGAAGGUGGACAAC
GCCGAGACCAUCAAGAAGGAGCUGGGCCUGAGCCUGACCGAGCCC
CUGAUGGAGCAGGUGGGCACCGAGGAGUUCAUCAAGAGGUUCGGC
GACGGCGCCAGCAGGGUGGUGCUGAGCCUGCCCUUCGCCGAGGGC
AGCAGCAGCGUGGAGUACAUCAACAACUGGGAGCAGGCCAAGGCC
CUGAGCGUGGAGCUGGAGAUCAACUUCGAGACCAGGGGCAAGAGG
GGCCAGGACGCCAUGUACGAGUACAUGGCCCAGGCCUGCGCCGGC
AACAGGGUGAGGAGGAGCGUGGGCAGCAGCCUGAGCUGCAUCAAC
CUGGACUGGGACGUGAUCAGGGACAAGACCAAGACCAAGAUCGAG
AGCCUGAAGGAGCACGGCCCCAUCAAGAACAAGAUGAGCGAGAGC
CCCAACAAGACCGUGAGCGAGGAGAAGGCCAAGCAGUACCUGGAG
GAGUUCCACCAGACCGCCCUGGAGCACCCCGAGCUGAGCGAGCUG
AAGACCGUGACCGGCACCAACCCCGUGUUCGCCGGCGCCAACUAC
GCCGCCUGGGCCGUGAACGUGGCCCAGGUGAUCGACAGCGAGACC
GCCGACAACCUGGAGAAGACCACCGCCGCCCUGAGCAUCCUGCCC
GGCAUCGGCAGCGUGAUGGGCAUCGCCGACGGCGCCGUGCACCAC
AACACCGAGGAGAUCGUGGCCCAGAGCAUCGCCCUGAGCAGCCUG
AUGGUGGCCCAGGCCAUCCCCCUGGUGGGCGAGCUGGUGGACAUC
GGCUUCGCCGCCUACAACUUCGUGGAGAGCAUCAUCAACCUGUUC
CAGGUGGUGCACAACAGCUACAACAGGCCCGCCUACAGCCCCGGC
CACAAGACCCAGCCCUUCCUGCACGACGGCUACGCCGUGAGCUGG
AACACCGUGGAGGACAGCAUCAUCAGGACCGGCUUCCAGGGCGAG
AGCGGCCACGACAUCAAGAUCACCGCCGAGAACACCCCCCUGCCC
AUCGCCGGCGUGCUGCUGCCCACCAUCCCCGGCAAGCUGGACGUG
AACAAGAGCAAGACCCACAUCAGCGUGAACGGCAGGAAGAUCAGG
AUGAGGUGCAGGGCCAUCGACGGCGACGUGACCUUCUGCAGGCCC
AAGAGCCCCGUGUACGUGGGCAACGGCGUGCACGCCAACCUGCAC
GUGGCCUUCCACAGGAGCAGCAGCGAAAAGAUCCACAGCAACGAG
AUCAGCAGCGACAGCAUCGGCGUGCUGGGCUACCAGAAGACCGUG
GACCACACCAAGGUGAACAGCAAGCUGAGCCUGUUCUUCGAGAUC
AAGAGCUAG

In one embodiment, the at least one RNA molecule of the invention encodes an amino acid sequence comprising or consisting of murine IL-4 (preferably having the sequence of SEQ ID NO: 11) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of murine IL-4 (preferably having the sequence of SEQ ID NO: 11) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of murine IL-4 (preferably having the sequence of SEQ ID NO: 11) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of murine IL-4 (preferably having the sequence of SEQ ID NO: 11) and TpD (preferably having the sequence of SEQ ID NO: 54) and optionally a spacer. In one embodiment, the at least one RNA molecule encodes a GP spacer. In one embodiment, the at least one RNA molecule further encodes a signal peptide (preferably having the sequence of SEQ ID NO: 140). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 140, murine IL-4 having preferably the sequence of SEQ ID NO: 11, a GP spacer and TpD having preferably the sequence of SEQ ID NO: 54.

In one embodiment, the at least one RNA molecule comprises or consists of a RNA sequence encoding murine IL-4 (preferably a RNA sequence of SEQ ID NO: 12) and TpD (preferably a RNA sequence of SEQ ID NO: 55) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 141). In one embodiment, the RNA molecule of the invention comprises or consists of, from 5′ to 3′: a RNA sequence encoding a signal peptide having preferably the sequence of SEQ ID NO: 141, a RNA sequence encoding murine IL-4 having preferably the sequence of SEQ ID NO: 12, a RNA sequence encoding a GP spacer having preferably the sequence GGCCCC and a RNA sequence encoding TpD having preferably the sequence of SEQ ID NO: 55.

In one embodiment, the at least one RNA molecule of the invention comprises or consists in the sequence of SEQ ID NO: 144.

In one embodiment, the at least one RNA molecule is a variant of SEQ ID NO: 144, wherein said variant present at least about 70%, 75%, 80%, 85%, 90%, 95% or more identity with the SEQ ID NO: 144 from which it derives.

SEQ ID NO: 144
AUGGGCCUGAACCCCCAGCUGGUGGUGAUCCUGCUGUUCUUCCUG
GAGUGCACCAGGAGCCACAUCCACGGCUGCGACAAGAACCACCUG
AGGGAGAUCAUCGGCAUCCUGAACGAGGUGACCGGCGAGGGCACC
CCCUGCACCGAGAUGGACGUGCCCAACGUGCUGACCGCCACCAAG
AACACCACCGAGAGCGAGCUGGUGUGCAGGGCCAGCAAGGUGCUG
AGGAUCUUCUACCUGAAGCACGGCAAGACCCCCUGCCUGAAGAAG
AACAGCAGCGUGCUGAUGGAGCUGCAGAGGCUGUUCAGGGCCUUC
AGGUGCCUGGACAGCAGCAUCAGCUGCACCAUGAACGAGAGCAAG
AGCACCAGCCUGAAGGACUUCCUGGAGAGCCUGAAGAGCAUCAUG
CAGAUGGACUACAGCGGCCCCAUCCUGAUGCAGUACAUCAAGGCC
AACAGCAAGUUCAUCGGCAUCCCCAUGGGCCUGCCCCAGAGCAUC
GCCCUGAGCAGCCUGAUGGUGGCCCAGUAG

In one embodiment, the at least one RNA molecule of the invention encodes an amino acid sequence comprising or consisting of murine IL-4 (preferably having the sequence of SEQ ID NO: 11) and p2-RR-p30 (TT) (SEQ ID NO: 56) and optionally a spacer.

In one embodiment, the at least one RNA molecule of the invention encodes an amino acid sequence comprising or consisting of murine IL-4 (preferably having the sequence of SEQ ID NO: 11) and a N10 construct (preferably having the sequence of SEQ ID NO: 80) and optionally a spacer.

In one embodiment, the at least one RNA molecule of the invention encodes an amino acid sequence comprising or consisting of murine IL-4 (preferably having the sequence of SEQ ID NO: 11) and a N19 construct (preferably having the sequence of SEQ ID NO: 82) and optionally a spacer. In one embodiment, the at least one RNA molecule encodes a GP spacer. In one embodiment, the at least one RNA molecule further encodes a signal peptide (preferably having the sequence of SEQ ID NO: 140). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 140, murine IL-4 having preferably the sequence of SEQ ID NO: 11, a GP spacer and a N19 construct having preferably the sequence of SEQ ID NO: 82.

In one embodiment, the at least one RNA molecule comprises or consists of a RNA sequence encoding murine IL-4 (preferably a RNA sequence of SEQ ID NO: 12) and a N19 construct (preferably having the sequence of SEQ ID NO: 83) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 141). In one embodiment, the RNA molecule of the invention comprises or consists of, from 5′ to 3′: a RNA sequence encoding a signal peptide having preferably the sequence of SEQ ID NO: 141, a RNA sequence encoding murine IL-4 having preferably the sequence of SEQ ID NO: 12, a RNA sequence encoding a GP spacer having preferably the sequence GGCCCC and a RNA sequence encoding a N19 construct having preferably the sequence of SEQ ID NO: 83.

In one embodiment, the at least one RNA molecule of the invention comprises or consists in the sequence of SEQ ID NO: 145.

In one embodiment, the at least one RNA molecule is a variant of SEQ ID NO: 145, wherein said variant present at least about 70%, 75%, 80%, 85%, 90%, 95% or more identity with the SEQ ID NO: 145 from which it derives.

SEQ ID NO: 145
AUGGGCCUGAACCCCCAGCUGGUGGUGAUCCUGCUGUUCUUCCUG
GAGUGCACCAGGAGCCACAUCCACGGCUGCGACAAGAACCACCUG
AGGGAGAUCAUCGGCAUCCUGAACGAGGUGACCGGCGAGGGCACC
CCCUGCACCGAGAUGGACGUGCCCAACGUGCUGACCGCCACCAAG
AACACCACCGAGAGCGAGCUGGUGUGCAGGGCCAGCAAGGUGCUG
AGGAUCUUCUACCUGAAGCACGGCAAGACCCCCUGCCUGAAGAAG
AACAGCAGCGUGCUGAUGGAGCUGCAGAGGCUGUUCAGGGCCUUC
AGGUGCCUGGACAGCAGCAUCAGCUGCACCAUGAACGAGAGCAAG
AGCACCAGCCUGAAGGACUUCCUGGAGAGCCUGAAGAGCAUCAUG
CAGAUGGACUACAGCGGCCCCGUGAGCAUCGACAAGUUCAGGAUC
UUCUGCAAGGCCAACCCCAAGAAGGGCCUGAAGUUCAUCAUCAAG
AGGUACACCCCCAACAACGAGAUCGACAGCAAGGGCAUCAGGGAG
GACAACAACAUCACCCUGAAGCUGGACAGGUGCAACAACAAGGGC
GAAAAGAAGAUCGCCAAGAUGGAGAAGGCCAGCAGCGUGUUCAAC
GUGGUGAACAAGGGCUUCAACAACUUCACCGUGAGCUUCUGGCUG
AGGGUGCCCAAGGUGAGCGCCAGCCACCUGGAGAAGGGCCAGUAC
AUCAAGGCCAACAGCAAGUUCAUCGGCAUCACCGAAAAGGGCCCC
CACCACACCGCCCUGAGGCAGGCCAUCCUGUGCUGGGGCGAGCUG
AUGACCCUGGCCAAGGGCCCCAAGUACGUGAAGCAGAACACCCUG
AAGCUGGCCACCAAGGGCUUCUUCCUGCUGACCAGGAUCCUGACC
AUCCCCCAGAGCCUGGACAAGGGCUACAGCGGCCCCCUGAAGGCC
GAGAUCGCCCAGAGGCUGGAGGACGUGAAGGGCGUGAGCAUCGAC
AAGUUCAGGAUCUUCUGCAAGGCCAACCCCAAGAAGGGCCUGAAG
UUCAUCAUCAAGAGGUACACCCCCAACAACGAGAUCGACAGCAAG
GGCAUCAGGGAGGACAACAACAUCACCCUGAAGCUGGACAGGUGC
AACAACAAGGGCGAAAAGAAGAUCGCCAAGAUGGAGAAGGCCAGC
AGCGUGUUCAACGUGGUGAACAAGGGCUUCAACAACUUCACCGUG
AGCUUCUGGCUGAGGGUGCCCAAGGUGAGCGCCAGCCACCUGGAG
AAGGGCCAGUACAUCAAGGCCAACAGCAAGUUCAUCGGCAUCACC
GAAAAGGGCCCCCACCACACCGCCCUGAGGCAGGCCAUCCUGUGC
UGGGGCGAGCUGAUGACCCUGGCCAAGGGCCCCAAGUACGUGAAG
CAGAACACCCUGAAGCUGGCCACCAAGGGCUUCUUCCUGCUGACC
AGGAUCCUGACCAUCCCCCAGAGCCUGGACAAGGGCUAG

In one embodiment, the at least one RNA molecule of the invention encodes an amino acid sequence comprising or consisting of murine IL-4 (preferably having the sequence of SEQ ID NO: 11) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and CRM₁₉₇ (SEQ ID NO: 48) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of murine IL-4 (preferably a peptide having or comprising a sequence selected from SEQ ID NO: 13, 14, 15 or 16) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of canine IL-4 (preferably having the sequence of SEQ ID NO: 21 and CRM₁₉₇ (SEQ ID NO: 48) and optionally a spacer.

In one embodiment, the at least one RNA molecule of the invention encodes an amino acid sequence comprising or consisting of canine IL-4 (preferably having the sequence of SEQ ID NO: 21) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of canine IL-4 (preferably having the sequence of SEQ ID NO: 21) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of canine IL-4 (preferably having the sequence of SEQ ID NO: 21) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of canine IL-4 (preferably having the sequence of SEQ ID NO: 21) and TpD (preferably having the sequence of SEQ ID NO: 54) and optionally a spacer.

In one embodiment, the at least one RNA molecule of the invention encodes an amino acid sequence comprising or consisting of canine IL-4 (preferably having the sequence of SEQ ID NO: 21) and p2-RR-p30 (TT) (SEQ ID NO: 56) and optionally a spacer.

In one embodiment, the at least one RNA molecule of the invention encodes an amino acid sequence comprising or consisting of canine IL-4 (preferably having the sequence of SEQ ID NO: 21) and a N10 construct (preferably having the sequence of SEQ ID NO: 80) and optionally a spacer.

In one embodiment, the at least one RNA molecule of the invention encodes an amino acid sequence comprising or consisting of canine IL-4 (preferably having the sequence of SEQ ID NO: 21) and a N19 construct (preferably having the sequence of SEQ ID NO: 82) and optionally a spacer.

In one embodiment, the at least one RNA molecule of the invention encodes an amino acid sequence comprising or consisting of canine IL-4 (preferably having the sequence of SEQ ID NO: 21) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and CRM₁₉₇ (SEQ ID NO: 48) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of canine IL-4 (preferably a peptide having or comprising a sequence selected from SEQ ID NO: 94, 95, 96 or 97) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of equine IL-4 (preferably having the sequence of SEQ ID NO: 99 and CRM₁₉₇ (SEQ ID NO: 48) and optionally a spacer.

In one embodiment, the at least one RNA molecule of the invention encodes an amino acid sequence comprising or consisting of equine IL-4 (preferably having the sequence of SEQ ID NO: 99) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of equine IL-4 (preferably having the sequence of SEQ ID NO: 99) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of equine IL-4 (preferably having the sequence of SEQ ID NO: 99) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of equine IL-4 (preferably having the sequence of SEQ ID NO: 99) and TpD (preferably having the sequence of SEQ ID NO: 54) and optionally a spacer.

In one embodiment, the at least one RNA molecule of the invention encodes an amino acid sequence comprising or consisting of equine IL-4 (preferably having the sequence of SEQ ID NO: 99) and p2-RR-p30 (TT) (SEQ ID NO: 56) and optionally a spacer.

In one embodiment, the at least one RNA molecule of the invention encodes an amino acid sequence comprising or consisting of equine IL-4 (preferably having the sequence of SEQ ID NO: 99) and a N10 construct (preferably having the sequence of SEQ ID NO: 80) and optionally a spacer.

In one embodiment, the at least one RNA molecule of the invention encodes an amino acid sequence comprising or consisting of equine IL-4 (preferably having the sequence of SEQ ID NO: 99) and a N19 construct (preferably having the sequence of SEQ ID NO: 82) and optionally a spacer.

In one embodiment, the at least one RNA molecule of the invention encodes an amino acid sequence comprising or consisting of equine IL-4 (preferably having the sequence of SEQ ID NO: 99) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and CRM₁₉₇ (SEQ ID NO: 48) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of equine IL-4 (preferably a peptide having or comprising a sequence selected from SEQ ID NO: 109, 110, 111, 112) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of human IL-13 (preferably having the sequence of SEQ ID NO: 23) and CRM₁₉₇ (SEQ ID NO: 48) and optionally a spacer. In one embodiment, the at least one RNA molecule encodes a GP spacer. In one embodiment, the at least one RNA molecule further encodes a signal peptide (preferably having the sequence of SEQ ID NO: 117). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 117, human IL-13 having preferably the sequence of SEQ ID NO: 23, a GP spacer and CRM₁₉₇ having preferably the sequence of SEQ ID NO: 48.

In one embodiment, the at least one RNA molecule comprises or consists of a RNA sequence encoding human IL-13 (preferably a RNA sequence of SEQ ID NO: 24) and CRM₁₉₇ (preferably a RNA sequence of SEQ ID NO: 49) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 118). In one embodiment, the RNA molecule of the invention comprises or consists of, from 5′ to 3′: a RNA sequence encoding a signal peptide having preferably the sequence of SEQ ID NO: 118, a RNA sequence encoding human IL-13 having preferably the sequence of SEQ ID NO: 24, a RNA sequence encoding a GP spacer having preferably the sequence GGCCCC and a RNA sequence encoding CRM₁₉₇ having preferably the sequence of SEQ ID NO: 49.

In one embodiment, the at least one RNA molecule of the invention encodes an amino acid sequence comprising or consisting of human IL-13 (preferably having the sequence of SEQ ID NO: 23) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of human IL-13 (preferably having the sequence of SEQ ID NO: 23) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of human IL-13 (preferably having the sequence of SEQ ID NO: 23) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of human IL-13 (preferably having the sequence of SEQ ID NO: 23) and TpD (preferably having the sequence of SEQ ID NO: 54) and optionally a spacer. In one embodiment, the at least one RNA molecule encodes a GP spacer. In one embodiment, the at least one RNA molecule further encodes a signal peptide (preferably having the sequence of SEQ ID NO: 117). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 117, human IL-13 having preferably the sequence of SEQ ID NO: 23, a GP spacer and TpD having preferably the sequence of SEQ ID NO: 54.

In one embodiment, the at least one RNA molecule comprises or consists of a RNA sequence encoding human IL-13 (preferably a RNA sequence of SEQ ID NO: 24) and TpD (preferably a RNA sequence of SEQ ID NO: 55) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 118). In one embodiment, the RNA molecule of the invention comprises or consists of, from 5′ to 3′: a RNA sequence encoding a signal peptide having preferably the sequence of SEQ ID NO: 118, a RNA sequence encoding human IL-13 having preferably the sequence of SEQ ID NO: 24, a RNA sequence encoding a GP spacer having preferably the sequence GGCCCC and a RNA sequence encoding TpD having preferably the sequence of SEQ ID NO: 55.

In one embodiment, the at least one RNA molecule of the invention encodes an amino acid sequence comprising or consisting of human IL-13 (preferably having the sequence of SEQ ID NO: 23) and p2-RR-p30 (TT) (SEQ ID NO: 56) and optionally a spacer.

In one embodiment, the at least one RNA molecule of the invention encodes an amino acid sequence comprising or consisting of human IL-13 (preferably having the sequence of SEQ ID NO: 23) and a N10 construct (preferably having the sequence of SEQ ID NO: 80) and optionally a spacer.

In one embodiment, the at least one RNA molecule of the invention encodes an amino acid sequence comprising or consisting of human IL-13 (preferably having the sequence of SEQ ID NO: 23) and a N19 construct (preferably having the sequence of SEQ ID NO: 82) and optionally a spacer. In one embodiment, the at least one RNA molecule encodes a GP spacer. In one embodiment, the at least one RNA molecule further encodes a signal peptide (preferably having the sequence of SEQ ID NO: 117). In one embodiment, the at least one RNA molecule of the invention encodes a single protein construct (preferably a single one-chain protein construct) comprising or consisting of, from N-terminal to C-terminal: a signal peptide having the sequence of SEQ ID NO: 117, human IL-13 having preferably the sequence of SEQ ID NO: 23, a GP spacer and a N19 construct having preferably the sequence of SEQ ID NO: 82.

In one embodiment, the at least one RNA molecule comprises or consists of a RNA sequence encoding human IL-13 (preferably a RNA sequence of SEQ ID NO: 24) and a N19 construct (preferably having the sequence of SEQ ID NO: 83) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 118). In one embodiment, the RNA molecule of the invention comprises or consists of, from 5′ to 3′: a RNA sequence encoding a signal peptide having preferably the sequence of SEQ ID NO: 118, a RNA sequence encoding human IL-13 having preferably the sequence of SEQ ID NO: 24, a RNA sequence encoding a GP spacer having preferably the sequence GGCCCC and a RNA sequence encoding a N19 construct having preferably the sequence of SEQ ID NO: 83.

In one embodiment, the at least one RNA molecule of the invention encodes an amino acid sequence comprising or consisting of human IL-13 (preferably having the sequence of SEQ ID NO: 23) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and CRM₁₉₇ (SEQ ID NO: 48) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of human IL-13 (preferably a peptide having or comprising a sequence selected from SEQ ID NO: 25, 26, 27 or 28) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of murine IL-13 (preferably having the sequence of SEQ ID NO: 33) and CRM₁₉₇ (SEQ ID NO: 48) and optionally a spacer. In one embodiment, the at least one RNA molecule encodes a GP spacer. In one embodiment, the at least one RNA molecule further encodes a signal peptide (preferably having the sequence of SEQ ID NO: 142). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 142, murine IL-13 having preferably the sequence of SEQ ID NO: 33, a GP spacer and CRM₁₉₇ having preferably the sequence of SEQ ID NO: 48.

In one embodiment, the at least one RNA molecule comprises or consists of a RNA sequence encoding murine IL-13 (preferably a RNA sequence of SEQ ID NO: 34) and CRM₁₉₇ (preferably a RNA sequence of SEQ ID NO: 49) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 143). In one embodiment, the RNA molecule of the invention comprises or consists of, from 5′ to 3′: a RNA sequence encoding a signal peptide having preferably the sequence of SEQ ID NO: 143, a RNA sequence encoding murine IL-13 having preferably the sequence of SEQ ID NO: 34, a RNA sequence encoding a GP spacer having preferably the sequence GGCCCC and a RNA sequence encoding CRM₁₉₇ having preferably the sequence of SEQ ID NO: 49.

In one embodiment, the at least one RNA molecule of the invention encodes an amino acid sequence comprising or consisting of murine IL-13 (preferably having the sequence of SEQ ID NO: 33) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of murine IL-13 (preferably having the sequence of SEQ ID NO: 33) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of murine IL-13 (preferably having the sequence of SEQ ID NO: 33) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of murine IL-13 (preferably having the sequence of SEQ ID NO: 33) and TpD (preferably having the sequence of SEQ ID NO: 54) and optionally a spacer. In one embodiment, the at least one RNA molecule encodes a GP spacer. In one embodiment, the at least one RNA molecule further encodes a signal peptide (preferably having the sequence of SEQ ID NO: 142). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 142, murine IL-13 having preferably the sequence of SEQ ID NO: 33, a GP spacer and TpD having preferably the sequence of SEQ ID NO: 54.

In one embodiment, the at least one RNA molecule comprises or consists of a RNA sequence encoding murine IL-13 (preferably a RNA sequence of SEQ ID NO: 34) and TpD (preferably a RNA sequence of SEQ ID NO: 55) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 143). In one embodiment, the RNA molecule of the invention comprises or consists of, from 5′ to 3′: a RNA sequence encoding a signal peptide having preferably the sequence of SEQ ID NO: 143, a RNA sequence encoding murine IL-13 having preferably the sequence of SEQ ID NO: 34, a RNA sequence encoding a GP spacer having preferably the sequence GGCCCC and a RNA sequence encoding TpD having preferably the sequence of SEQ ID NO: 55.

In one embodiment, the at least one RNA molecule of the invention encodes an amino acid sequence comprising or consisting of murine IL-13 (preferably having the sequence of SEQ ID NO: 33) and p2-RR-p30 (TT) (SEQ ID NO: 56) and optionally a spacer.

In one embodiment, the at least one RNA molecule of the invention encodes an amino acid sequence comprising or consisting of murine IL-13 (preferably having the sequence of SEQ ID NO: 33) and a N10 construct (preferably having the sequence of SEQ ID NO: 80) and optionally a spacer.

In one embodiment, the at least one RNA molecule of the invention encodes an amino acid sequence comprising or consisting of murine IL-13 (preferably having the sequence of SEQ ID NO: 33) and a N19 construct (preferably having the sequence of SEQ ID NO: 82) and optionally a spacer. In one embodiment, the at least one RNA molecule encodes a GP spacer. In one embodiment, the at least one RNA molecule further encodes a signal peptide (preferably having the sequence of SEQ ID NO: 142). In one embodiment, the at least one RNA molecule of the invention encodes a single protein construct (preferably a single one-chain protein construct) comprising or consisting of, from N-terminal to C-terminal: a signal peptide having preferably the sequence of SEQ ID NO: 142, murine IL-13 having preferably the sequence of SEQ ID NO: 33, a GP spacer and a N19 construct having preferably the sequence of SEQ ID NO: 82.

In one embodiment, the at least one RNA molecule comprises or consists of a RNA sequence encoding murine IL-13 (preferably a RNA sequence of SEQ ID NO: 34) and a N19 construct (preferably having the sequence of SEQ ID NO: 83) and optionally a spacer. In one embodiment, the at least one RNA molecule further comprises a sequence encoding a GP spacer (preferably a RNA sequence GGCCCC). In one embodiment, the at least one RNA molecule further comprises a sequence encoding a signal peptide (preferably a RNA sequence of SEQ ID NO: 143). In one embodiment, the RNA molecule of the invention comprises or consists of, from 5′ to 3′: a RNA sequence encoding a signal peptide having preferably the sequence of SEQ ID NO: 143, a RNA sequence encoding murine IL-13 having preferably the sequence of SEQ ID NO: 34, a RNA sequence encoding a GP spacer having preferably the sequence GGCCCC and a RNA sequence encoding a N19 construct having preferably the sequence of SEQ ID NO: 83.

In one embodiment, the at least one RNA molecule of the invention encodes an amino acid sequence comprising or consisting of murine IL-13 (preferably having the sequence of SEQ ID NO: 33) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and CRM₁₉₇ (SEQ ID NO: 48) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of murine IL-13 (preferably a peptide having or comprising a sequence selected from SEQ ID NO: 35, 36, 37 or 38) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of canine IL-13 (preferably having the sequence of SEQ ID NO: 43) and CRM₁₉₇ (SEQ ID NO: 48) and optionally a spacer.

In one embodiment, the at least one RNA molecule of the invention encodes an amino acid sequence comprising or consisting of canine IL-13 (preferably having the sequence of SEQ ID NO: 43) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of canine IL-13 (preferably having the sequence of SEQ ID NO: 43) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of canine IL-13 (preferably having the sequence of SEQ ID NO: 43) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of canine IL-13 (preferably having the sequence of SEQ ID NO: 43) and TpD (preferably having the sequence of SEQ ID NO: 54) and optionally a spacer.

In one embodiment, the at least one RNA molecule of the invention encodes an amino acid sequence comprising or consisting of canine IL-13 (preferably having the sequence of SEQ ID NO: 43) and p2-RR-p30 (TT) (SEQ ID NO: 56) and optionally a spacer.

In one embodiment, the at least one RNA molecule of the invention encodes an amino acid sequence comprising or consisting of canine IL-13 (preferably having the sequence of SEQ ID NO: 43) and a N10 construct (preferably having the sequence of SEQ ID NO: 80) and optionally a spacer.

In one embodiment, the at least one RNA molecule of the invention encodes an amino acid sequence comprising or consisting of canine IL-13 (preferably having the sequence of SEQ ID NO: 43) and a N19 construct (preferably having the sequence of SEQ ID NO: 82) and optionally a spacer.

In one embodiment, the at least one RNA molecule of the invention encodes an amino acid sequence comprising or consisting of canine IL-13 (preferably having the sequence of SEQ ID NO: 43) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and CRM₁₉₇ (SEQ ID NO: 48) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of canine IL-13 (preferably a peptide having or comprising a sequence selected from SEQ ID NO: 119, 120, 121 or 122) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of equine IL-13 (preferably having the sequence of SEQ ID NO: 101) and CRM₁₉₇ (SEQ ID NO: 48) and optionally a spacer.

In one embodiment, the at least one RNA molecule of the invention encodes an amino acid sequence comprising or consisting of equine IL-13 (preferably having the sequence of SEQ ID NO: 101) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of equine IL-13 (preferably having the sequence of SEQ ID NO: 101) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of equine IL-13 (preferably having the sequence of SEQ ID NO: 101) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of equine IL-13 (preferably having the sequence of SEQ ID NO: 101) and TpD (preferably having the sequence of SEQ ID NO: 54) and optionally a spacer.

In one embodiment, the at least one RNA molecule of the invention encodes an amino acid sequence comprising or consisting of equine IL-13 (preferably having the sequence of SEQ ID NO: 101) and p2-RR-p30 (TT) (SEQ ID NO: 56) and optionally a spacer.

In one embodiment, the at least one RNA molecule of the invention encodes an amino acid sequence comprising or consisting of equine IL-13 (preferably having the sequence of SEQ ID NO: 101) and a N10 construct (preferably having the sequence of SEQ ID NO: 80) and optionally a spacer.

In one embodiment, the at least one RNA molecule of the invention encodes an amino acid sequence comprising or consisting of equine IL-13 (preferably having the sequence of SEQ ID NO: 101) and a N19 construct (preferably having the sequence of SEQ ID NO: 82) and optionally a spacer.

In one embodiment, the at least one RNA molecule of the invention encodes an amino acid sequence comprising or consisting of equine IL-13 (preferably having the sequence of SEQ ID NO: 101) and at least one CD4 polyepitope (preferably having a sequence selected from the group consisting of SEQ ID NO: 58-67) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and CRM₁₉₇ (SEQ ID NO: 48) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and CRM₁₉₇₍₂₉₉₋₃₁₂₎ (SEQ ID NO: 50) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and CRM₁₉₇₍₄₂₅₋₄₄₀₎ (SEQ ID NO: 52) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and CRM₁₉₇₍₃₀₀₋₄₅₀₎ (SEQ ID NO: 46) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and TpD (preferably having the sequence of SEQ ID NO: 54) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and p2-RR-p30 (TT) (SEQ ID NO: 56) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and a N10 construct (preferably having the sequence of SEQ ID NO: 80) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and a N19 construct (preferably having the sequence of SEQ ID NO: 82) and optionally a spacer.

In one embodiment, the at least one RNA molecule of the invention encodes an amino acid sequence comprising or consisting of a peptide comprising at least one epitope of equine IL-13 (preferably a peptide having or comprising a sequence selected from SEQ ID NO: 129, 130, 131 or 132) and at least one CD4 polyepitope (preferably having a sequence selected from the group consisting of SEQ ID NO: 58-67) and optionally a spacer.

In one embodiment, the at least one RNA molecule of the invention comprises chemical structures with the ability to promote stability and/or translation efficiency may also be used. In one embodiment the at least one RNA molecule comprises preferably a 5′ and 3′ UTRs. In one embodiment, the 5′ UTR is between zero and 3000 nucleotides in length. The 5′ and 3′ UTRs can be the naturally occurring, endogenous 5′ and 3′ UTRs for the gene to be expressed (e.g., IL-4 or IL-13). Alternatively, UTR sequences that are not endogenous to the gene of interest can be added by incorporating the UTR sequences into the forward and reverse primers or by any other modifications of the template. The use of UTR sequences that are not endogenous to the gene to be expressed can be useful for modifying the stability and/or translation efficiency of the RNA. For example, it is known that AU-rich elements in 3′ UTR sequences can decrease the stability of mRNA.

In one embodiment, the 5′ UTR can contain the Kozak sequence of the endogenous gene. In one embodiment, the 5′ UTR can be derived from an RNA virus whose RNA genome is stable in cells.

In one embodiment, the at least one RNA molecule of the invention has both a cap on the 5′ end and a 3′ poly(A) tail which determine ribosome binding, initiation of translation and stability mRNA in the cell.

In one embodiment, the at least one RNA molecule of the invention comprises a poly(A) tail. Poly(A) tails provide stability to RNAs and reduce their degradation. Generally, the length of a poly(A) tail positively correlates with the stability of the RNA. In one embodiment, the poly(A) tail is between 100 and 5000 adenosines.

Additionally, the attachment of different chemical groups to the 3′ end can increase RNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

In one embodiment, the at least one RNA molecule of the present invention comprises a 5′ cap. 5′ caps on RNAs may provide stability to RNA molecules.

In one embodiment, the at least one RNA molecule of the present invention comprises an internal ribosome entry site (IRES) sequence, in particular a viral, chromosomal or artificially designed sequence which initiates cap-independent ribosome binding to mRNA and facilitates the initiation of translation.

The GC content play a role in the regulation of mRNA stability or also to reduce secondary structures. Enrichment of GC content may optimize and increase the stability of the mRNA and also protein translation in vivo. In one embodiment, the at least one RNA molecule of the present invention comprises an optimized GC content. In one embodiment, the at least one RNA molecule comprises a high GC content.

In one embodiment, the RNA sequences are modified to replace at least one, preferably all, uridine residues by 1-methylpseudouridine residues.

In one embodiment, the RNA is not a naked RNA and is formulated for delivery into target cells.

The composition may be delivered into the target cells by well-known methods such as lipofection, sonoporation, microinjection, biolistics, virosomes, liposomes, lipid nanoparticles, immunoliposomes, polycation or lipid:nucleic acid conjugates, virus-like particles and artificial virions.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid/RNA associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. In one embodiment, the delivery vehicle is a lipid nanoparticle, that may be referred to as a LNP.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL).

“Liposome” is a generic term encompassing a variety of single and multilamellar lipid vehicles formed by the generation of enclosed lipid bilayers or aggregates. Liposomes can be characterized as having vesicular structures with a phospholipidbilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes and lipidots.

In one embodiment, the composition is a pharmaceutical composition and further comprises at least one pharmaceutically acceptable excipient. Consequently, the present invention further relates to a pharmaceutical composition comprising, consisting essentially of or consisting of at least one RNA molecule, wherein the at least one RNA molecule encodes an amino acid sequence of at least one cytokine or cytokine fragment, at least one T cell epitope and optionally at least one spacer, and at least one pharmaceutically acceptable excipient.

Pharmaceutically acceptable excipients that may be used in the pharmaceutical composition of the invention include, but are not limited to, ion exchangers, alumina, aluminum stearate, lecithin, serum proteins, such as, for example, human serum albumin, buffer substances such as, for example, phosphates, glycine, sorbic acid, potassium sorbate, partial glyceride mixtures of saturated vegetable fatty acids, water, salts or electrolytes, such as, for example, protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the composition of the present invention is, or is for use as, a medicament. Consequently, the present invention further relates to a medicament comprising, consisting essentially of or consisting of at least one RNA molecule, wherein the at least one RNA molecule encodes at least one cytokine or cytokine fragment, at least one T cell epitope and optionally at least one spacer as described hereinabove.

The present invention further relates to at least one RNA molecule for use as a medicament, wherein the at least one RNA molecule encodes at least one cytokine or cytokine fragment, at least one T cell epitope and optionally at least one spacer.

As used herein, the term “consisting essentially of”, with reference to a pharmaceutical composition or medicament, means that the composition or the at least one RNA molecule of the invention is the only one therapeutic agent or agent with a biologic activity within said pharmaceutical composition or medicament.

In one embodiment, the pharmaceutical composition or medicament of the invention comprises or consists essentially of at least one RNA molecule encoding IL-4 or a fragment thereof and at least one T cell epitope optionally linked by at least one spacer.

In one embodiment, the pharmaceutical composition or medicament of the invention comprises or consists essentially of at least one RNA molecule encoding IL-13 or a fragment thereof and at least one T cell epitope optionally linked by at least one spacer.

In one embodiment, the pharmaceutical composition or medicament of the invention comprises or consists essentially of at least one RNA molecule encoding both IL-4 and IL-13 (or fragments thereof) and at least one T cell epitope optionally linked by a spacer. In one embodiment, the pharmaceutical composition or medicament of the invention comprises or consists essentially of (i) at least one RNA molecule encoding IL-4 (or a fragment thereof) and at least one T cell epitope optionally linked by a spacer and (ii) at least one RNA molecule encoding IL-13 (or a fragment thereof) and at least one T cell epitope optionally linked by a spacer.

In one embodiment, the composition comprising the at least one RNA molecule of the invention is a vaccine composition. In one embodiment of the invention, the vaccine composition of the invention comprises at least one adjuvant. In one embodiment of the invention, the vaccine composition of the invention does not comprise any adjuvant.

This invention further relates to a formulation of the composition, pharmaceutical composition, medicament or vaccine of the invention, wherein the composition, pharmaceutical composition, medicament or vaccine is adjuvanted. In one embodiment, the composition, pharmaceutical composition, medicament or vaccine of the invention thus comprise one or more adjuvants.

In one embodiment, the at least one RNA molecule, the composition, pharmaceutical composition, or vaccine composition of the invention may be administered (or is for administration) by injection, topically (such as, for example, by transdermal delivery), rectally, nasally or vaginally.

In one embodiment, the at least one RNA molecule, composition, pharmaceutical composition, medicament, or vaccine composition of the invention is in an adapted form for an injection. Thus, in one embodiment, the composition, pharmaceutical composition, medicament, or vaccine composition of the invention is to be injected (or is for injection) to the subject by intramuscular, intraperitoneal, or subcutaneous injection.

Examples of forms suitable for injectable use include, but are not limited to, sterile solutions or dispersions and sterile powders for the extemporaneous preparation of sterile injectable solutions or dispersions. The prevention against contamination by microorganisms can be brought about by adding in the composition preservatives such as, for example, various antibacterial and antifungal agents (for example, parabens, chlorobutanol, phenol, sorbic acid, thimerosal and the like). In an embodiment, it may be preferable to include isotonic agents, for example, sugars or sodium chloride, to reduce pain during injection. In one embodiment, prolonged absorption of the injectable compositions can be brought about by the use in the compositions of agents delaying absorption, for example, aluminum monostearate and gelatin.

In one embodiment, a lyophilized vaccine composition of the invention is solubilized in water for injection and gently mixed; then an immunoadjuvant as described hereinabove, is added; the mixture is gently mixed and charged into a suitable syringe.

This invention thus also relates to a medical device, including a syringe filled or prefilled with a vaccine composition of the invention.

In one embodiment, the at least one RNA molecule, composition, pharmaceutical composition, medicament, or vaccine composition of the invention is in an adapted form for topical administration. Examples of forms adapted for topical administration include, without being limited to, polymeric patch, or controlled-release patch, and the like.

In another embodiment, the at least one RNA molecule, composition, pharmaceutical composition, medicament, or vaccine composition of the invention is in an adapted form for rectal administration. Examples of forms adapted for rectal administration include, without being limited to, suppository, micro enemas, enemas, gel, rectal foam, cream, ointment, and the like.

The present invention further relates to at least one RNA molecule of the invention, or to a composition, pharmaceutical composition, medicament, or vaccine composition of the invention, for treating an inflammatory disorder in a subject.

The present invention thus further relates to a method for treating an inflammatory disorder in a subject, comprising administering to the subject the at least one RNA molecule, composition, pharmaceutical composition, medicament or a vaccine composition of the invention.

The present invention further relates to a method for inducing an immune response against IL-4, IL-13 or both in a subject, comprising administering to the subject the at least one RNA molecule, composition, pharmaceutical composition, medicament, or vaccine composition of the invention.

The present invention further relates to a method for inducing in a subject the production of antibodies that inhibits the biological activity or neutralizes the biological activity of IL-4, IL-13 or both, comprising administering to the subject the at least one RNA molecule, composition, pharmaceutical composition, medicament, or vaccine composition of the invention. In one embodiment, the antibodies are polyclonal antibodies.

In one embodiment, the subject is affected, preferably is diagnosed, with an inflammatory disorder, in particular with a disorder associated with aberrant IL-4 and/or IL-13 expression or activity.

In one embodiment, the subject is a human. Preferably, according to this embodiment, the at least one cytokine comprised in the composition of the invention is human.

In one embodiment, the subject in need thereof is a non-human animal, including, but not limited to, a farm animal—or an animal of agricultural value (such as, e.g., cattle, cows, bison, pigs, swine, sheep, goats, horses, donkeys, alpacas, llamas, deer, elks, moose, ostriches, emus, ducks, geese, chickens, partridges, quails, pheasants, minks, salmons, codfishes, catfishes, herrings, trout, basses, perches, flounders, sharks, tuna fishes, cancers, lobsters, crayfishes, snails, clams, oysters, and the like), a companion animal (such as, e.g., dog, cats, rabbits, rodents, fishes, snakes and the like), and a non-human primate (such as, e.g., great apes including chimpanzees, gorillas, and orangutans; lesser apes, including gibbons; Old World monkeys; New World monkeys; and prosimians, including tarsiers, lemurs, and lorises).

In one embodiment, the inflammatory disorder is a disorder associated with aberrant IL-4 and/or IL-13 expression or activity.

Examples of inflammatory disorder include, but are not limited to, asthma (either allergic or non-allergic), allergic conditions (such as, for example, food allergies, venom allergy, cat allergy, drug allergy, hyper IgE syndrome, allergic rhinitis, allergic conjunctivitis and allergic enterogastritis), atopic disorders (such as, for example, atopic dermatitis, urticaria (including chronic idiopathic urticaria and chronic spontaneous urticaria), eczema), bullous pemphigoid, respiratory disorders (such as allergic and nonallergic asthma, chronic obstructive pulmonary disease (COPD)), nasal polyposis and other conditions involving airway inflammation (such as, for example, eosinophilia, fibrosis and excess mucus production including cystic fibrosis and pulmonary fibrosis, systemic sclerosis (SSc)); inflammatory and/or autoimmune disorders or conditions, gastrointestinal disorders or conditions (such as, for example, inflammatory bowel diseases (IBD) and eosinophilic esophagitis (EE), and eosinophilic-mediated gastrointestinal disease, ulcerative colitis, Crohn's disease and systemic lupus erythematosus); systemic lupus erythematosus, liver disorders or conditions (such as, for example, cirrhosis, and hepatocellular carcinoma), scleroderma; fibrotic diseases or disorders (such as, for example, fibrosis of the liver (such as, for example, fibrosis caused by a hepatitis B and/or C virus)), scleroderma; solid tumors or cancers such as leukemia (such as, for example, B cell chronic lymphocytic leukaemia), glioblastoma, lymphoma (such as, for example, Hodgkin's lymphoma) and mastocytosis.

In one embodiment, the inflammatory disorder is selected from the group comprising asthma (e.g., allergic asthma), atopic dermatitis, chronic obstructive pulmonary disease (COPD), pulmonary fibrosis, food allergy, nasal polyposis and eosinophilic esophagitis.

In one embodiment, the inflammatory disorder is selected from the group comprising asthma (e.g., allergic asthma), atopic dermatitis, chronic obstructive pulmonary disease (COPD), pulmonary fibrosis and food allergy.

In one embodiment, the inflammatory disorder is allergy, asthma, or atopic dermatitis.

In one embodiment, the inflammatory disorder is allergic asthma.

In one embodiment, the inflammatory disorder is a solid tumor. In one embodiment, the method of the present invention is for preventing metastasis from solid tumor.

In one embodiment, the treatment consists of a single dose or a plurality of doses over a period of time.

In one embodiment of the invention, the subject to be treated is administrated at least once with the therapeutically effective amount of the composition as described here above.

In another embodiment of the invention, the subject to be treated may be further administrated with a therapeutically effective amount of the composition as described here above when the amount of antibodies against IL-4 is undetectable in a serum sample obtained from the subject.

In another embodiment of the invention, the subject to be treated may be further administrated with a therapeutically effective amount of the composition as described here above when the amount of antibodies against IL-13 is undetectable in a serum sample obtained from the subject.

In another embodiment of the invention, the subject to be treated may be further administrated with a therapeutically effective amount of the composition as described here above when the amount of antibodies against IL-4 and IL-13 are undetectable in a serum sample obtained from the subject.

### BRIEF DESCRIPTION OF THE DRAWINGS

FIGS. 1 and 2 are maps of IL-4 RNA constructs according to the present invention. EP: epitope; EPX: presence of multiple epitopes (X epitopes are present); FL: full length.

FIGS. 3 and 4 are maps of IL-13 RNA constructs of the present invention. EP: epitope; EPX: presence of multiple epitopes (X epitopes are present); FL: full length.

FIG. 5 is a combination of graphs showing the in vitro protein expression of muIL4 and CRM in transfected HEK293 cells. The protein expression was measured with flow cytometry at 24 h post LNP/mRNAs transfection. LNP/mRNA-muIL4 (white box), LNP/mRNA-CRM (hatched box), LNP/mRNA-muIL4-TpD (clear grey box), LNP/mRNA-muIL4-N19 (dark grey) and LNP/mRNA-muIL4-CRM (black box) were tested. FIG. 5A is a graph showing the percentage of cells expressing muIL4. FIG. 5B is a graph showing the percentage of cells expressing CRM. FIG. 5C is a graph showing the median of fluorescence intensity (MFI) of muIL4 in the expressed cells. (n=2) Mean with range.

FIG. 6 shows the in vitro activity of muIL4 in supernatant of HEK293 cells transfected with the at least one RNA construct of the present invention. Protein activity was measured with a proliferative bioassay in HEK293 cells supernatants at 24 h post LNP/mRNAs transfection. Supernatants of mRNA-muIL4 (white), mRNA-muIL4-TpD (clear grey), mRNA-muIL4-CRM (black), mRNA-muIL4-N19 (dark grey), and mRNA-CRM (hatched circle) transfections were tested. Optical densities (OD) at 490 nm in function of the sample dilution are presented. (n=2) Mean with range.

FIG. 7 shows the scheme of in vivo study. BALB/c mice received three intramuscular (IM) immunizations at DO, D7 and D28 of 10 g LNP/mRNA (arrows). Blood samples were harvested at indicted time points.

FIG. 8 shows the induction of neutralizing antibodies against muIL4 in sera samples following intramuscular injection of LNP/mRNA in BALB/c mice. BALB/c mice received three IM immunizations at DO, D7 and D28 of 10 g mRNA-muIL4 (white circle), mRNA-CRM (hatched circle), mRNA-muIL4-TpD (clear grey circle), mRNA-muIL4-N19 (dark grey circle) or mRNA-muIL4-CRM (black circle) within LNP. Bars indicate median. The neutralizing capacities demonstrated by the half-maximal neutralizing titers against muIL4 were determined at D21, D41, D62 and D80 after the first administration. n=10 per group for CRM, muIL4-N19 and muIL4-CRM. n=9 for muIL4-TpD and n=2 for muIL4.

FIG. 9 shows animal survival curve following intramuscular injection of LNP/mRNA in BALB/c mice. BALB/c mice received three IM immunizations at DO, D7 and D28 of 10 μg mRNA-muIL4 (discontinued line), mRNA-muIL4-TpD (grey line), mRNA-CRM, mRNA-muIL4-N19 and mRNA-muIL4-CRM (black line) within LNP. Kaplan-Mayer survival curves are shown (n=10/group).

### EXAMPLES

### RNA Constructions

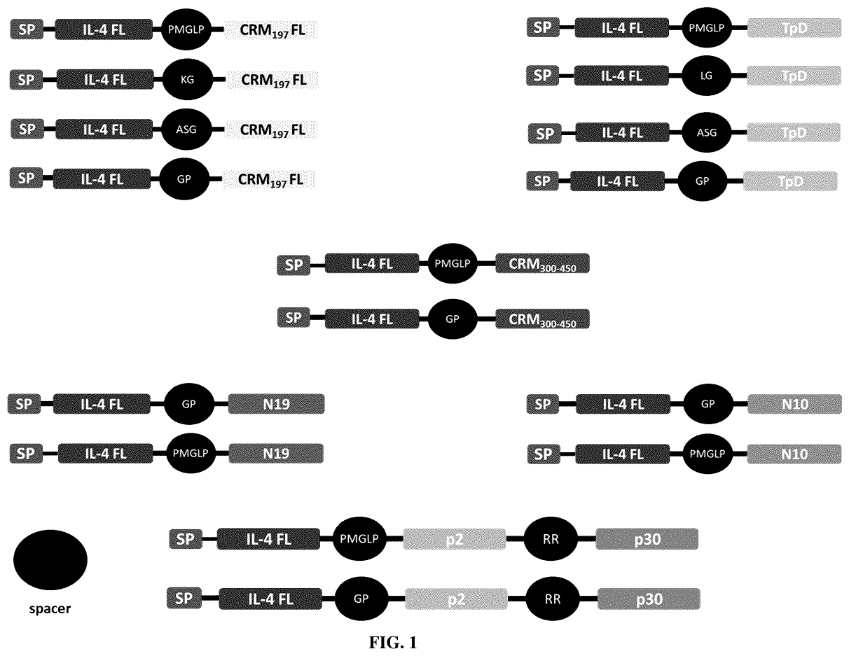

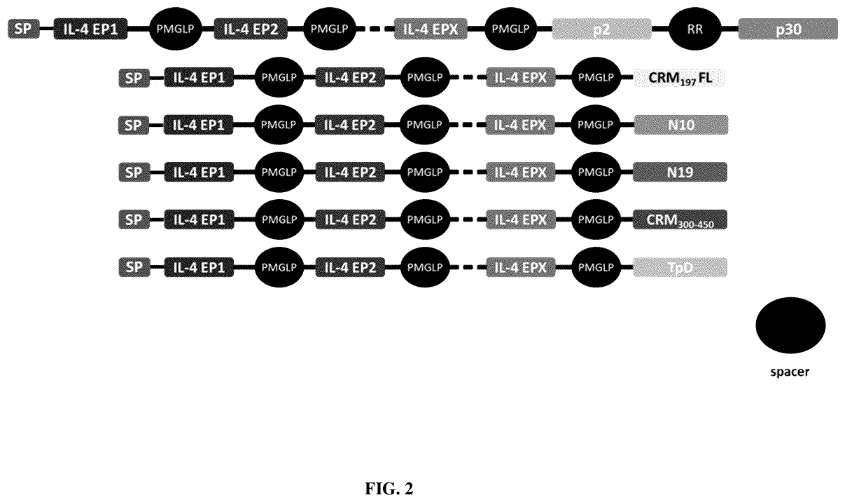

Constructs according to the present invention, corresponding to RNA molecules encoding IL-4 (full length or epitopes thereof), a spacer and at least one T cell epitope are presented in FIGS. 1 and 2 and Tables 4 and 5.

TABLE 4
RNA constructs encoding IL-4 full length cytokine

| Cytokine | spacer | T cell epitope(s) |
|---|---|---|
| IL-4 FL | PMGLP | CRM₁₉₇ FL |
| IL-4 FL | KG | CRM₁₉₇ FL |
| IL-4 FL | ASG | CRM₁₉₇ FL |
| IL-4 FL | GP | CRM₁₉₇ FL |
| IL-4 FL | PMGLP | TpD |
| IL-4 FL | LG | TpD |
| IL-4 FL | ASG | TpD |
| IL-4 FL | GP | TpD |
| IL-4 FL | PMGLP | CRM₁₉₇₍₃₀₀₋₄₅₀₎ |
| IL-4 FL | GP | CRM₁₉₇₍₃₀₀₋₄₅₀₎ |
| IL-4 FL | GP | N19 |
| IL-4 FL | PMGLP | N19 |
| IL-4 FL | GP | N10 |
| IL-4 FL | PMGLP | N10 |
| IL-4 FL | PMGLP | P2-RR-p30 |
| IL-4 FL | GP | P2-RR-p30 |

FL: full length

TABLE 5
RNA constructs encoding IL-4 epitopes

| | | | | | | T cell |
|---|---|---|---|---|---|---|
| Cytokine | spacer | Cytokine | spacer | Cytokine | spacer | epitope(s) |
| IL-4 EP1 | PMGLP | IL-4 EP2 | PMGLP | IL-4 EPX | PMGLP | P2-RR-p30 |
| IL-4 EP1 | PMGLP | IL-4 EP2 | PMGLP | IL-4 EPX | PMGLP | CRM₁₉₇ FL |
| IL-4 EP1 | PMGLP | IL-4 EP2 | PMGLP | IL-4 EPX | PMGLP | N10 |
| IL-4 EP1 | PMGLP | IL-4 EP2 | PMGLP | IL-4 EPX | PMGLP | N19 |
| IL-4 EP1 | PMGLP | IL-4 EP2 | PMGLP | IL-4 EPX | PMGLP | CRM₁₉₇₍₃₀₀₋₄₅₀₎ |
| IL-4 EP1 | PMGLP | IL-4 EP2 | PMGLP | IL-4 EPX | PMGLP | TpD |

EP: epitope;
EPX: X represents the number of epitopes;
FL: full length

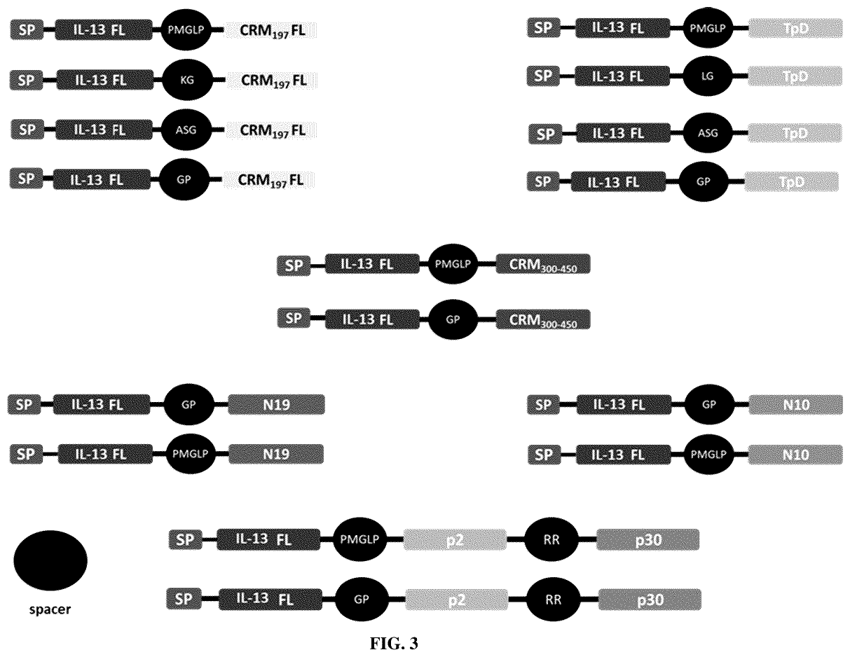

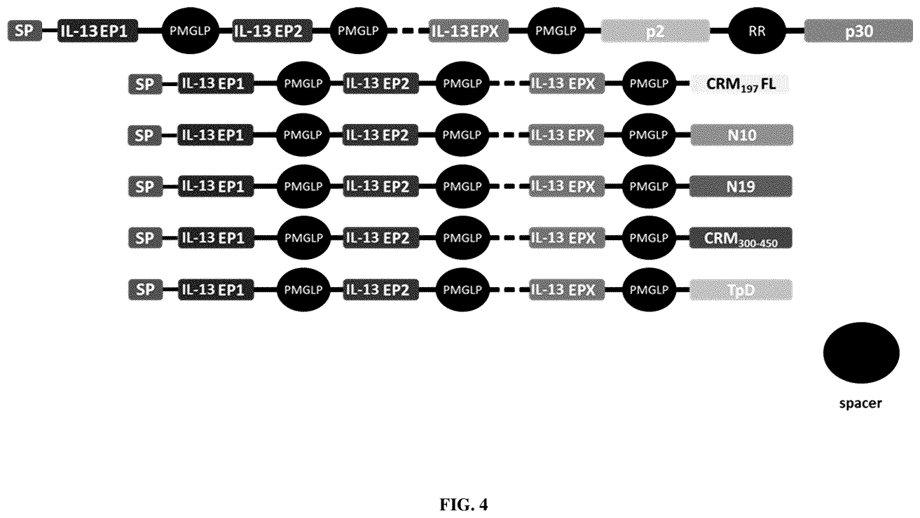

Constructs according to the present invention, corresponding to RNA molecules encoding IL-13 (full length or epitopes thereof), a spacer and at least one T cell epitope are presented in FIGS. 3 and 4 and Tables 6 and 7.

TABLE 6
RNA constructs encoding IL-13 full length cytokine

| Cytokine | spacer | T cell epitope(s) |
|---|---|---|
| IL-13 FL | PMGLP | CRM₁₉₇ FL |
| IL-13 FL | KG | CRM₁₉₇ FL |
| IL-13 FL | ASG | CRM₁₉₇ FL |
| IL-13 FL | GP | CRM₁₉₇ FL |
| IL-13 FL | PMGLP | TpD |
| IL-13 FL | LG | TpD |
| IL-13 FL | ASG | TpD |
| IL-13 FL | GP | TpD |
| IL-13 FL | PMGLP | CRM₁₉₇₍₃₀₀₋₄₅₀₎ |
| IL-13 FL | GP | CRM₁₉₇₍₃₀₀₋₄₅₀₎ |
| IL-13 FL | GP | N19 |
| IL-13 FL | PMGLP | N19 |
| IL-13 FL | GP | N10 |
| IL-13 FL | PMGLP | N10 |
| IL-13 FL | PMGLP | P2-RR-p30 |
| IL-13 FL | GP | P2-RR-p30 |

FL: full length

TABLE 7
RNA constructs encoding IL-13 epitopes

| | | | | | | T cell |
|---|---|---|---|---|---|---|
| Cytokine | spacer | Cytokine | spacer | Cytokine | spacer | epitope(s) |
| IL-13 | PMGLP | IL-13 | PMGLP | IL-13 | PMGLP | P2-RR-p30 |
| EP1 | | EP2 | | EPX | | |
| IL-13 | PMGLP | IL-13 | PMGLP | IL-13 | PMGLP | CRM₁₉₇ FL |
| EP1 | | EP2 | | EPX | | |
| IL-13 | PMGLP | IL-13 | PMGLP | IL-13 | PMGLP | N10 |
| EP1 | | EP2 | | EPX | | |
| IL-13 | PMGLP | IL-13 | PMGLP | IL-13 | PMGLP | N19 |
| EP1 | | EP2 | | EPX | | |
| IL-13 | PMGLP | IL-13 | PMGLP | IL-13 | PMGLP | CRM₁₉₇₍₃₀₀₋₄₅₀₎ |
| EP1 | | EP2 | | EPX | | |
| IL-13 | PMGLP | IL-13 | PMGLP | IL-13 | PMGLP | TpD |
| EP1 | | EP2 | | EPX | | |

EP: epitope;
EPX: X represents the number of epitopes;
FL: full length

### Example 1

### Material and Methods

### Mice

The IL4RA/IL13/IL4 humanized mouse line (hIL-4/hIL-13KI; hIL-4RαKI) and female BALB/cJRj mice were used in the following experiments (Conde et al., 2021).

Intranasal Challenges with IL-4 and IL-13.

hIL-4/hIL-13KI; hIL-4RαKI mice were exposed to the mRNA constructs of the invention, comprising sequences encoding IL-4 or IL-13, or PBS as a control. After sacrifice, an analysis of BAL eosinophil numbers was performed.

### Quantification of Antibodies Against Mouse and Human IL-4 and IL-13, and T Cell Epitopes.

The immunogenicity of the mRNA vaccines of the present invention was assessed by evaluating antibodies against mouse IL-4, human IL-4, mouse IL-13, human IL-13, and T cell epitope(s) in sera collected at different time points after vaccination, using ELISA systems.

Assessment of the Neutralizing Capacity Against IL-4 and IL-13 in Sera from Vaccinated Mice.

Neutralizing capacities of the anti-mouse IL-4, anti-mouse IL-13, anti-human IL-4, and anti-human IL-13 antibodies were evaluated using for example the CTLL-2 cells proliferation assay (ECACC, ref. 93042610, batch number: 12K006.) or a HEK-Blue IL-4/IL-13 reporter gene cell line bioassay (InvivoGen, hkb-il413, batch number: X14-37-01), adapted from the manufacturer's instructions.

### Example 2

### Material and Methods

In Vitro Functionality of mRNA

3 μg of mRNA encoding murine IL4 (mu-IL4), CRM, or fusion proteins muIL4-GP-CRM, muIL4-GP-TpD or muIL4-GP-N19 encapsulated in LNP was diluted in OptiMEM serum free medium and added to 6×10⁵ HEK293 cells in complete medium. After overnight incubation, cells were harvested, fixed and permeabilized with Inside stain kit (Miltenyi). Intracellular expression of muIL4 and CRM₁₉₇ was evaluated by staining cells with a monoclonal anti-mouse IL-4 PE-conjugated (clone BVD4-1D11) and with an anti-diphteria toxin IgG1 (clone 8G1) detected by an anti-mouse IgG1-APC conjugated. The expression was assessed with flow cytometry.

In Vitro Activity of muIL4 in Supernatant of Transfected HEK293 Cells.

Supernatants of HEK293 cells transfected with mRNA of the present invention were collected at 24 h post transfection and muIL4 proliferating activity was assessed using a CTLL-2 cell assay. Briefly, CTLL-2 cells were grown in complete RPMI-1640 medium in the presence of human IL-2 at 10 ng/mL. For activity bioassays, human IL-2 was replaced by potential mu-IL4 present in supernatant. Therefore, potential mu-IL4 induces CTLL-2 growth.

Supernatant samples were added at a chosen dilution and 2-fold serially diluted in RPMI-1640 medium+FBS [10% (v/v)] in culture plates. Mu-IL4 protein was used as a positive control and standard curve beginning at 10 ng/ml. Then 20,000 CTLL-2 cells were added to all wells. After 48 h of incubation, cell viability was quantified by MTS/PMS assay by adding 40 μl of the substrate. The optical density (OD) was read after 4 h incubation at 490 nm.

In Vivo Generation of Neutralizing Antibodies Against muIL4 Following Intramuscular Injection of LNP/mRNA.

Five weeks old female BALB/c ByJ mice were obtained from Janviers labs (Saint Berthevin, France). A pre-dosing bleed was performed seven days before the first immunization. Animals were injected by intramuscular (IM) route with 50 μl of LNPs containing 10 g of mRNA encoding muIL4, CRM or fusion proteins muIL4-GP-TpD, muIL4-GP-N19 and muIL4-GP-CRM at day 0, day 7 and day 28. Blood was collected at days 21, 41, 62, and 80. Neutralizing capacity against muIL-4 was assessed using a bioassay test performed on CTLL-2 cells.

In brief, cells were grown in presence of human IL 2, and for neutralization bioassays, human IL 2 was replaced by mu-IL4. Therefore, potential anti-muIL-4 neutralizing antibodies induced after immunization will prevent CTLL-2 growth.

Serum samples were added at 1/200 final dilution, and positive control polyclonal anti-mu-IL4 antibody (AF-404-NA, Biotechne) at 1 g/mL final concentration. All samples were 2-fold serially diluted in RPMI-1640 medium+FBS [10% (v/v)]. Mu-IL4 was added at 2 ng/mL to serum samples and control, and then incubated for 1 h at room temperature. Then, 20,000 CTLL-2 cells were added to pre-incubated samples (serum or positive control with muIL-4). After 48 h of incubation, cell viability was quantified by MTS/PMS assay.

NC50 results were expressed as the serum dilution factor (dil-1) neutralizing 50% of mu-IL4 activity which maximum is defined by the incubation of mu-IL4 at 2 ng/mL with CTLL-2 cells. The NC50 is determined by interpolating the serum dilution resulting in a 50% of mu-IL4 activity.

### Constructs

Five different constructs of mRNA were used. Murine IL-4 was coupled to three different T cell epitopes: TpD (SEQ ID NO: 144), N19 (SEQ ID NO: 145) and CRM₁₉₇ (SEQ ID NO: 146). RNA-IL4 (SEQ ID NO: 147) and RNA-CRM (SEQ ID NO: 148) were both used as control.

SEQ ID NO: 147
AUGGGCCUGAACCCCCAGCUGGUGGUGAUCCUGCUGUUCUUCCUG
GAGUGCACCAGGAGCCACAUCCACGGCUGCGACAAGAACCACCUG
AGGGAGAUCAUCGGCAUCCUGAACGAGGUGACCGGCGAGGGCACC
CCCUGCACCGAGAUGGACGUGCCCAACGUGCUGACCGCCACCAAG
AACACCACCGAGAGCGAGCUGGUGUGCAGGGCCAGCAAGGUGCUG
AGGAUCUUCUACCUGAAGCACGGCAAGACCCCCUGCCUGAAGAAG
AACAGCAGCGUGCUGAUGGAGCUGCAGAGGCUGUUCAGGGCCUUC
AGGUGCCUGGACAGCAGCAUCAGCUGCACCAUGAACGAGAGCAAG
AGCACCAGCCUGAAGGACUUCCUGGAGAGCCUGAAGAGCAUCAUG
CAGAUGGACUACAGCUAG
SEQ ID NO: 148
AUGGGCCUGAACCCCCAGCUGGUGGUGAUCCUGCUGUUCUUCCUG
GAGUGCACCAGGAGCGGCGCCGACGACGUGGUGGACAGCAGCAAG
AGCUUCGUGAUGGAGAACUUCAGCAGCUACCACGGCACCAAGCCC
GGCUACGUGGACAGCAUCCAGAAGGGCAUCCAGAAGCCCAAGAGC
GGCACCCAGGGCAACUACGACGACGACUGGAAGGAGUUCUACAGC
ACCGACAACAAGUACGACGCCGCCGGCUACAGCGUGGACAACGAG
AACCCCCUGAGCGGCAAGGCCGGCGGCGUGGUGAAGGUGACCUAC
CCCGGCCUGACCAAGGUGCUGGCCCUGAAGGUGGACAACGCCGAG
ACCAUCAAGAAGGAGCUGGGCCUGAGCCUGACCGAGCCCCUGAUG
GAGCAGGUGGGCACCGAGGAGUUCAUCAAGAGGUUCGGCGACGGC
GCCAGCAGGGUGGUGCUGAGCCUGCCCUUCGCCGAGGGCAGCAGC
AGCGUGGAGUACAUCAACAACUGGGAGCAGGCCAAGGCCCUGAGC
GUGGAGCUGGAGAUCAACUUCGAGACCAGGGGCAAGAGGGGCCAG
GACGCCAUGUACGAGUACAUGGCCCAGGCCUGCGCCGGCAACAGG
GUGAGGAGGAGCGUGGGCAGCAGCCUGAGCUGCAUCAACCUGGAC
UGGGACGUGAUCAGGGACAAGACCAAGACCAAGAUCGAGAGCCUG
AAGGAGCACGGCCCCAUCAAGAACAAGAUGAGCGAGAGCCCCAAC
AAGACCGUGAGCGAGGAGAAGGCCAAGCAGUACCUGGAGGAGUUC
CACCAGACCGCCCUGGAGCACCCCGAGCUGAGCGAGCUGAAGACC
GUGACCGGCACCAACCCCGUGUUCGCCGGCGCCAACUACGCCGCC
UGGGCCGUGAACGUGGCCCAGGUGAUCGACAGCGAGACCGCCGAC
AACCUGGAGAAGACCACCGCCGCCCUGAGCAUCCUGCCCGGCAUC
GGCAGCGUGAUGGGCAUCGCCGACGGCGCCGUGCACCACAACACC
GAGGAGAUCGUGGCCCAGAGCAUCGCCCUGAGCAGCCUGAUGGUG
GCCCAGGCCAUCCCCCUGGUGGGCGAGCUGGUGGACAUCGGCUUC
GCCGCCUACAACUUCGUGGAGAGCAUCAUCAACCUGUUCCAGGUG
GUGCACAACAGCUACAACAGGCCCGCCUACAGCCCCGGCCACAAG
ACCCAGCCCUUCCUGCACGACGGCUACGCCGUGAGCUGGAACACC
GUGGAGGACAGCAUCAUCAGGACCGGCUUCCAGGGCGAGAGCGGC
CACGACAUCAAGAUCACCGCCGAGAACACCCCCCUGCCCAUCGCC
GGCGUGCUGCUGCCCACCAUCCCCGGCAAGCUGGACGUGAACAAG
AGCAAGACCCACAUCAGCGUGAACGGCAGGAAGAUCAGGAUGAGG
UGCAGGGCCAUCGACGGCGACGUGACCUUCUGCAGGCCCAAGAGC
CCCGUGUACGUGGGCAACGGCGUGCACGCCAACCUGCACGUGGCC
UUCCACAGGAGCAGCAGCGAAAAGAUCCACAGCAACGAGAUCAGC
AGCGACAGCAUCGGCGUGCUGGGCUACCAGAAGACCGUGGACCAC
ACCAAGGUGAACAGCAAGCUGAGCCUGUUCUUCGAGAUCAAGAGC
UAG

### Results

In Vitro Protein Expression of muIL4 and CRM in Transfected HEK293 Cells

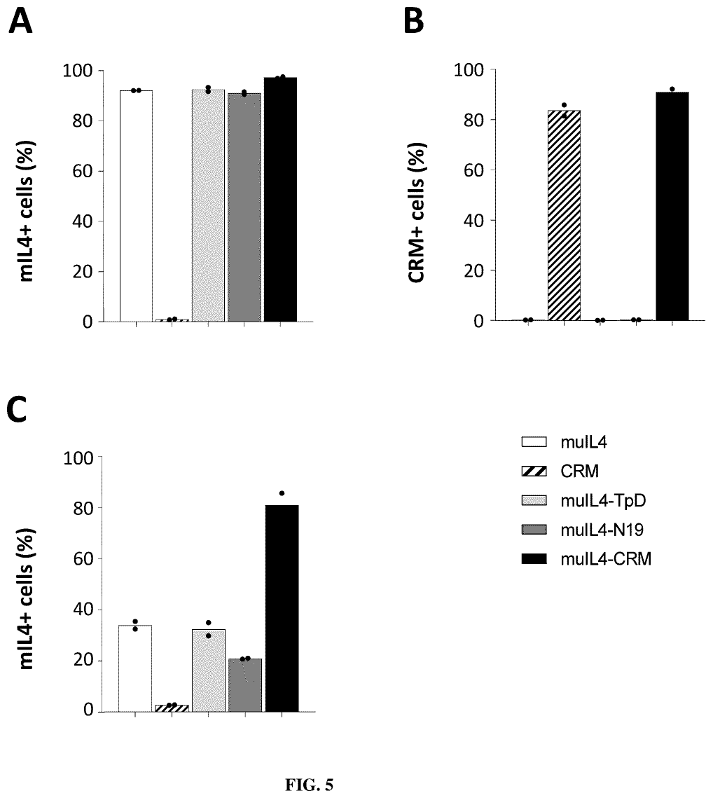

The muIL4 expression was observed in all tested conditions, except in the condition where mRNA-CRM alone was transfected (hatched box), with a percentage of cells expressing IL4 of above 80% (FIG. 5A). The CRM expression was observed in CRM and muIL4-CRM conditions (FIG. 5B). The MFI of muIL4 in HEK293 cells was 2 to 4-fold higher with muIL4-CRM (black box) as compared to muIL4, muIL4-TpD and muIL4-N19 (FIG. 5C).

In Vitro Activity of muIL4 in Supernatant of Transfected HEK293 Cells

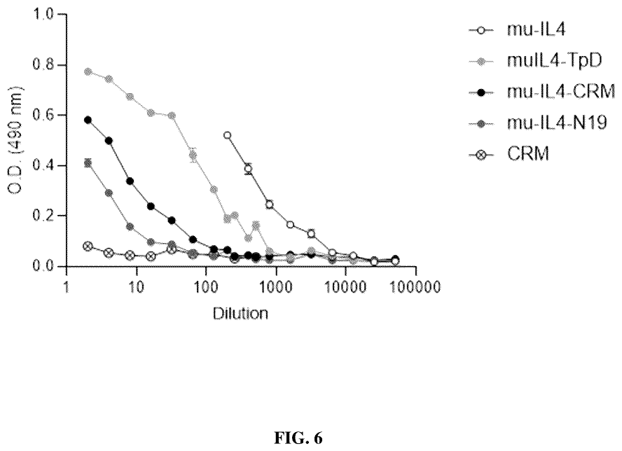

As shown in FIG. 6, the highest mu-IL-4 activity was observed with the cells transfected with mRNA-IL4. The muIL4 activities measured in the supernatant of HEK293 cells transfected with mRNA-muIL4-N19, mRNA-muIL4-CRM, mRNA-muIL4-TpD were lower. Finally, no mu-IL4 activity was detected in the supernatant of HEK293 cells transfected with mRNA-CRM. As observed by western blot (data not shown), substantially equivalent quantities of mIL4 proteins were quantified in the supernatant of transfected HEK293 cells in the different conditions (except for the mRNA-CRM condition). Thus, in vitro differences in muIL4 activity observed in FIG. 6 are not due to differences in the muIL4 protein quantity, but instead to a lower activity of the proteins obtained with the RNA constructs of the present invention as compared to native murine IL4.

Analysis of Neutralizing Antibodies Against muIL4 in Serum Samples Following Intramuscular Injection of LNP/mRNA in BALB/c Mice

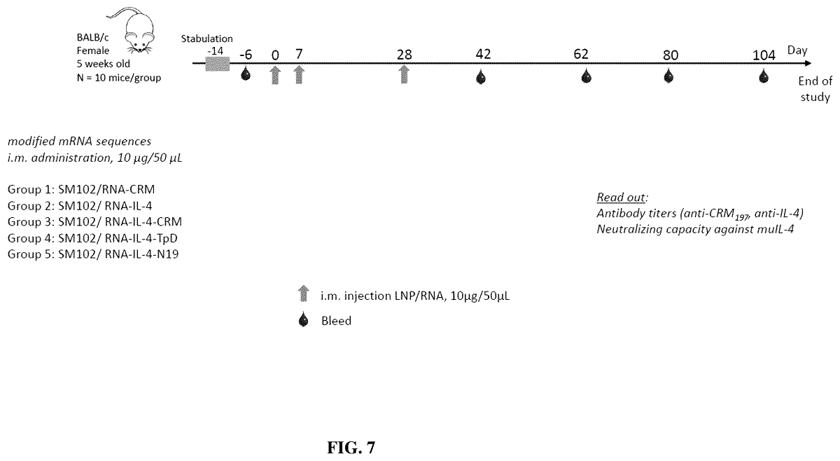

Five weeks old female BALB/c ByJ mice were injected by intramuscular (IM) route with 50 μl of LNPs containing 10 g of mRNA encoding muIL4, CRM or fusion proteins muIL4-GP-TpD, muIL4-GP-N19 and muIL4-GP-CRM at day 0, day 7 and day 28. Blood was collected at days 21, 41, 62, 80 and at the end of study (day 104) (FIG. 7) and the presence of neutralizing antibodies was evaluated in the serum of these mice.

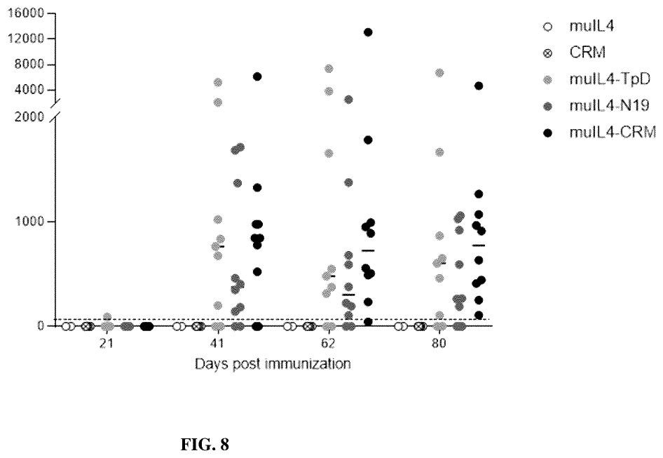

Neutralizing antibodies against muIL4 were detected from Day 41 in mice immunized three times with 10 g of mRNA encoding muIL4-TpD, muIL4-N19 and muIL4-CRM within LNP. At day 41 with 7/9, 6/10 and 8/10 responders were detected in groups administered with mRNA encoding muIL4-TpD, muIL4-N19 and muIL4-CRM within LNP respectively (NC50>200 dil-1). At day 62, although one more mouse was found positive in muIL4-CRM group, the median started decreasing for all groups, presumably indicating that the peak of response was reached around day 41. Except for high neutralizing capacity mice, NC50 did not decrease between day 62 and day 80, medians were roughly equivalent between day 62 and day 80: from 726 to 775 for muIL4-CRM group, 302 to 266 for muIL4-N19 group and from 481 to 605 for muIL4-TpD group. No muIL4 neutralizing antibodies were detected in muIL4 and CRM groups at any time (FIG. 8 and Table 8).

TABLE 8
Median NC50 (Dil-1) of muIL4 neutralizing capacities
for CRM, muIL4, muIL4-CRM, muIL4-N19 and muIL4-
CRM groups at days 21, 41, 62 and 80.

| | muIL4 | CRM | muIL4-TpD | muIL4-N19 | muIL4-CRM |
|---|---|---|---|---|---|
| Day 21 | 0 | 0 | 0 | 0 | 0 |
| Day 41 | 0 | 0 | 766 | 377.5 | 846.5 |
| Day 62 | 0 | 0 | 481 | 302 | 726 |
| Day 80 | 0 | 0 | 605 | 266 | 775 |

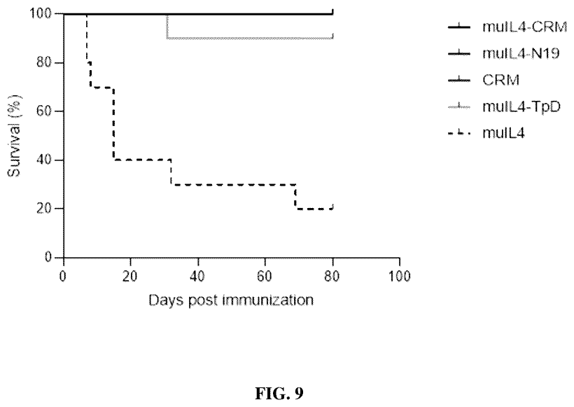

All the animals injected with LNPs encapsulating mRNA-CRM, mRNA-muIL4-CRM and mRNA-muIL4-N19 were alive (100%) at day 80. The survival of mice receiving mRNA-muIL4-TpD within LNP drops to 90% due to the death of one isolated mouse. Finally, only 20% of survival was observed in the group receiving mRNA-muIL4 within LNP at day 80 with 60% of mice dying after two rounds of immunizations (DO and D7) (FIG. 9). This result shows a toxicity of the mRNA-muIL4, that was not observed with the mRNA constructs as described in the present invention.

1-15. (canceled)

16. A composition comprising at least one RNA molecule, wherein the at least one RNA molecule encodes at least one amino acid sequence comprising:
at least one cytokine, or at least one fragment or epitope thereof,
at least one T cell epitope, and
optionally at least one spacer.

17. The composition according to claim 16, wherein the at least one cytokine is interleukin-4 (IL-4) and/or interleukin-13 (IL-13).

18. The composition according to claim 16, wherein the at least one cytokine is IL-4.

19. The composition according to claim 17, wherein the IL-4 fragment is selected from the group consisting of SEQ ID NOs: 7-10, 13-16, 94-97 and 109-112.

20. The composition according to claim 16, wherein the at least one cytokine is IL-13.

21. The composition according to claim 17, wherein the IL-13 fragment is selected from the group consisting of SEQ ID NOs: 25-28, 35-38, 119-122 and 129-132.

22. The composition according to claim 16, wherein the at least one RNA molecule encodes IL-4, or at least one fragment or epitope thereof, and IL-13, or at least one fragment or epitope thereof.

23. The composition according to claim 16, wherein the at least one T cell epitope is selected from the group consisting of CRM₁₉₇, combination of diphteria and tetanus epitopes (TpD), epitopes of tetanus toxin (TT), universal CD4 polyepitopes, variants and fragments thereof.

24. The composition according to claim 16, wherein the at least one spacer is selected from the group consisting of PMGLP, cathepsin cleavage sites, amino acids doublets, GP, GPGPG, GGSGGGGSGG, (GGGGS)ₙ wherein n ranges from 1 to 4, LG, ASG, KG and RR.

25. The composition according to claim 16, wherein the at least one RNA molecule is encapsulated.

26. The composition according to claim 16, wherein the at least one RNA molecule is encapsulated in a nanoparticle, in a liposome or in a virus-like particle.

27. A pharmaceutical composition comprising the composition according to claim 16, and at least one pharmaceutically acceptable excipient.

28. A vaccine composition comprising the composition according to claim 16, and optionally at least one adjuvant.

29. A medicament comprising at least one RNA molecule, wherein the at least one RNA molecule encodes at least one amino acid sequence comprising:
at least one cytokine, or at least one fragment or epitope thereof,
at least one T cell epitope, and
optionally at least one spacer.

30. A method for treating an inflammatory disorder in a subject in need thereof, comprising administering to the subject a composition according to claim 16.

31. The method according to claim 30, wherein said disorder is associated with aberrant IL-4 and/or IL-13 expression or activity.

32. The method according to claim 30, wherein the inflammatory disorder is selected from the group consisting of asthma, allergic conditions, atopic disorders, bullous pemphigoid, respiratory disorders, nasal polyposis and other conditions involving airway inflammation; inflammatory and/or autoimmune disorders or conditions, gastrointestinal disorders or conditions; systemic lupus erythematosus, liver disorders or conditions, scleroderma; fibrotic diseases or disorders; solid tumors or cancers and mastocytosis.

33. The method according to claim 30, wherein the inflammatory disorder is selected from the group consisting of allergic asthma, non-allergic asthma, food allergies, venom allergy, allergy to animals, drug allergy, anaphylaxis, hyper IgE syndrome, allergic rhinitis, allergic conjunctivitis, allergic enterogastritis, atopic dermatitis, urticaria, eczema, chronic obstructive pulmonary disease (COPD), eosinophilia, fibrosis, excess mucus production, systemic sclerosis (SSc), inflammatory bowel diseases (IBD), eosinophilic esophagitis (EE), eosinophilic-mediated gastrointestinal disease, ulcerative colitis, Crohn's disease, cirrhosis, hepatocellular carcinoma, fibrosis of the liver, leukemia, glioblastoma, and lymphoma.

34. The method according to claim 30, wherein the inflammatory disorder is selected from the group consisting of chronic idiopathic urticaria, chronic spontaneous urticaria, cystic fibrosis, pulmonary fibrosis, fibrosis caused by hepatitis B virus, fibrosis caused by hepatitis C virus, B cell chronic lymphocytic leukemia, and Hodgkin's lymphoma.

35. The method according to claim 30, wherein the inflammatory disorder is selected from the group consisting of asthma, atopic dermatitis, chronic obstructive pulmonary disease (COPD), pulmonary fibrosis, food allergy, nasal polyposis and eosinophilic esophagitis.
